SEQUENCE LISTING (18 sequences)

SEQ ID NO: 1 - DNA, 4385 bp, adeno-associated virus 9
cagagagggagtggccaactccatcactaggggtaatcgcgaagcgcctcccacgctgccgcgtcagcgctgacgtagattacgtcataggggagtggtcctgtattagctgtcacgtgagtgcttttgcgacattttgcgacaccacatggccatttgaggtatatatggccgagtgagcgagcaggatctccattttgaccgcgaaatttgaacgagcagcagccatgccgggcttctacgagattgtgatcaaggtgccgagcgacctggacgagcacctgccgggcatttctgactcttttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggagaagctggtccagaccatctaccgcgggatcgagccgaccctgcccaactggttcgcggtgaccaagacgcgtaatggcgccggcggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtaggcccttcacttccggtggacattacgcagaaccgcatctaccgcatcctgcagctcaacggctacgaccctgcctacgccggctccgtctttctcggctgggcacaaaagaagttcgggaaacgcaacaccatctggctgtttgggccggccaccacgggaaagaccaacatcgcagaagccattgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccactcccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgcttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacggcaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtaattcctcgggaaattggcattgcgattccacatggctgggggacagagtcatcaccaccagcacccgaacctgggcattgcccacctacaacaaccacctctacaagcaaatctccaatggaacatcgggaggaagcaccaacgacaacacctactttggctacagcaccccctgggggtattttgacttcaacagattccactgccacttctcaccacgtgactggcagcgactcatcaacaacaactggggattccggccaaagagactcaacttcaagctgttcaacatccaggtcaaggaggttacgacgaacgaaggcaccaagaccatcgccaataaccttaccagcaccgtccaggtctttacggactcggagtaccagctaccgtacgtcctaggctctgcccaccaaggatgcctgccaccgtttcctgcagacgtcttcatggttcctcagtacggctacctgacgctcaacaatggaagtcaagcgttaggacgttcttctttctactgtctggaatacttcccttctcagatgctgagaaccggcaacaactttcagttcagctacactttcgaggacgtgcctttccacagcagctacgcacacagccagagtctagatcgactgatgaaccccctcatcgaccagtacctatactacctggtcagaacacagacaactggaactgggggaactcaaactttggcattcagccaagcaggccctagctcaatggccaatcaggctagaaactgggtacccgggccttgctaccgtcagcagcgcgtctccacaaccaccaaccaaaataacaacagcaactttgcgtggacgggagctgctaaattcaagctgaacgggagagactcgctaatgaatcctggcgtggctatggcatcgcacaaagacgacgaggaccgcttctttccatcaagtggcgttctcatatttggcaagcaaggagccgggaacgatggagtcgactacagccaggtgctgattacagatgaggaagaaattaaagccaccaaccctgtagccacagaggaatacggagcagtggccatcaacaaccaggccgctaacacgcaggcgcaaactggacttgtgcataaccagggagttattcctggtatggtctggcagaaccgggacgtgtacctgcagggccctatttgggctaaaatacctcacacagatggcaactttcacccgtctcctctgatgggtggatttggactgaaacacccacctccacagattctaattaaaaatacaccagtgccggcagatcctcctcttaccttcaatcaagccaagctgaactctttcatcacgcagtacagcacgggacaagtcagcgtggaaatcgagtgggagctgcagaaagaaaacagcaagcgctggaatccagagatccagtatacttcaaactactacaaatctacaaatgtggactttgctgtcaataccaaaggtgtttactctgagcctcgccccattggtactcgttacctcacccgtaatttgtaattgcctgttaatcaataaaccggttaattcgtttcagttgaactttggtctctgcg

SEQ ID NO: 2 - DNA, 1185 bp, Gallus gallus
ttcccagatagccgccggcacccaccgctccgtgggacctcggcacaggtagccaagcatgtcggactctgaagaggtcgttgaggaatacgagcaggagcaggaagaggagtatgtggaagaagaagaggaagaatggcttgaggaagacgacggtcaggaggatcaggtagacgaggaggaagaggagacagaggaaaccacggcagaagaacaagaagatgaaacaaaagcaccaggagaaggtggtgagggggaccgggagcaggagcctggggaaggtgaatcaaagcccaaacccaagcccttcatgcccaacctggtgcctcccaaaatccctgatggcgagcgcctggatttcgatgacatccaccgcaagcgcatggagaaggacctgaatgagctgcaggccctcatcgaagcccattttgagagcaggaagaaggaggaagaggagctcatctctctcaaggacaggattgagcagcggagggcagagagggcagagcagcagcgcatccgcagcgagagggagaaggagcgccaggcccgcatggctgaggagagagctcgcaaagaggaagaggaggcacggaagaaggctgagaaagaagctcggaaaaagaaagctttctccaacatgctgcactttggaggctacatgcagaagtcggagaagaagggtggcaagaagcaaacggagcgggagaagaagaaaaagatcctcagcgagcggcggaagcctctgaacatcgaccacctcagcgaagacaaactgagggacaaagccaaggagctgtggcaaaccatccgtgacctggaggctgagaaatttgacttgcaggagaagttcaagcggcagaagtacgagatcaacgtccttcgaaatcgtgtcagtgaccaccagaaggtcaaagggtcaaaggctgcccgtgggaagaccatggtgggcggccggtggaagtagatggctctgaaggcaaaggtgaggctcagccatcagatgcagtgctgtgcgctcaacctatgccagggctctgctgcctccccaccatgcagtgcttgtacagtgcttgctgctggctccacgctgccggggtgggcaggtgctcagcgaggcgctgattctcatctccacacccccacatgatgttgtgtctgtaaataaagagaggagtgaggggg

SEQ ID NO: 3 - protein, 302 aa, Gallus gallus
MSDSEEVVEEYEQEQEEEYVEEEEEEWLEEDDGQEDQVDEEEEETEETTAEEQEDETKAPGEGGEGDREQEPGEGESKPKPKPFMPNLVPPKIPDGERLDFDDIHRKRMEKDLNELQALIEAHFESRKKEEEELISLKDRIEQRRAERAEQQRIRSEREKERQARMAEERARKEEEEARKKAEKEARKKKAFSNMLHFGGYMQKSEKKGGKKQTEREKKKKILSERRKPLNIDHLSEDKLRDKAKELWQTIRDLEAEKFDLQEKFKRQKYEINVLRNRVSDHQKVKGSKAARGKTMVGGRWK

SEQ ID NO: 4 - DNA, 3357 bp, Mus musculus
ccatcctggtctatagagagagttccagaacagccagggctacagataaacccatctggaaaaacaaagttgaatgacccaagaggggttctcagagggtggcgtgtgctccctggcaagcctatgacatggccggggcctgcctctctctgcctctgaccctcagtggctcccatgaactccttgcccaatggcatctttttcctgcgctccttgggttattccagtctcccctcagcattccttcctcagggcctcgctcttctctctgctccctccttgcacagctggctctgtccacctcagatgtcacagtgctctctcagaggaggaaggcaccatgtaccctctgtttcccaggtaagggttcaatttttaaaaatggttttttgtttgtttgtttgtttgtttgtttgtttgtttttcaagacagggctcctctgtgtagtcctaactgtcttgaaactccctctgtagaccaggtcgacctcgaactcttgaaacctgccacggaccacccagtcaggtatggaggtccctggaatgagcgtcctcgaagctaggtgggtaagggttcggcggtgacaaacagaaacaaacacagaggcagtttgaatctgagtgtattttgcagctctcaagcaggggattttatacataaaaaaaaaaaaaaaaaaaaaaccaaacattacatctcttagaaactatatccaatgaaacaatcacagataccaaccaaaaccattgggcagagtaaagcacaaaaatcatccaagcattacaactctgaaaccatgtattcagtgaatcacaaacagaacaggtaacatcattattaatataaatcaccaaaatataacaattctaaaaggatgtatccagtgggggctgtcgtccaaggctagtggcagatttccaggagcaggttagtaaatcttaaccactgaactaactctccagccccatggtcaattattatttagcatctagtgcctaatttttttttataaatcttcactatgtaatttaaaactattttaattcttcctaattaaggctttctttaccatataccaaaattcacctccaatgacacacgcgtagccatatgaaattttattgttgggaaaatttgtacctatcataatagttttgtaaatgatttaaaaagcaaagtgttagccgggcgtggtggcacacgcctttaatccctgcactcgggaggcaggggcaggaggatttctgagtttgaggccagcctggtctacagagtgagttccaggacagccagggctacacagagaaaccctgtctcgaaccccccaccccccaaaaaaagcaaagtgttggtttccttggggataaagtcatgttagtggcccatctctaggcccatctcacccattattctcgcttaagatcttggcctaggctaccaggaacatgtaaataagaaaaggaataagagaaaacaaaacagagagattgccatgagaactacggctcaatattttttctctccggcgaagagttccacaaccatctccaggaggcctccacgttttgaggtcaatggcctcagtctgtggaacttgtcacacagatcttactggaggtggtgtggcagaaacccattccttttagtgtcttgggctaaaagtaaaaggcccagaggaggcctttgctcatctgaccatgctgacaaggaacacgggtgccaggacagaggctggaccccaggaacaccttaaacacttcttcccttctccgccccctagagcaggctcccctcaccagcctgggcagaaatgggggaagatggagtgaagccatactggctactccagaatcaacagagggagccgggggcaatactggagaagctggtctccccccaggggcaatcctggcacctcccaggcagaagaggaaacttccacagtgcatctcacttccatgaatcccctcctcggactctgaggtccttggtcacagctgaggtgcaaaaggctcctgtcatattgtgtcctgctctggtctgccttccacagcttgggggccacctagcccacctctccctagggatgagagcagccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatccagggtgaggggcaggctgagggcggccacttccctcagccgcaggtttgttttcccaagaatggtttttctgcttctgtagcttttcctgtcaattctgccatggtggagcagcctgcactgggcttctgggagaaaccaaaccgggttctaacctttcagctacagttattgcctttcctgtagatgggcgactacagccccacccccacccccgtctcctgtatccttcctgggcctggggatcctaggctttcactggaaatttccccccaggtgctgtaggctagagtcacggctcccaagaacagtgcttgcctggcatgcatggttctgaacctccaactgcaaaaaatgacacataccttgacccttggaaggctgaggcagggggattgccatgagtgcaaagccagactgggtggcatagttagaccctgtctcaaaaaaccaaaaacaattaaataactaaagtcaggcaagtaatcctactcgggagactgaggcagagggattgttacatgtctgaggccagcctggactacatagggtttcaggctagccctgtctacagagtaaggccctatttcaaaaacacaaacaaaatggttctcccagctgctaatgctcaccaggcaatgaagcctggtgagcattagcaatgaaggcaatgaaggagggtgctggctacaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 5 - DNA, 20 bp, Artificial Sequence
  synthetic
agaactgcctgcgtgagatt

SEQ ID NO: 6 - DNA, 20 bp, Artificial Sequence
  synthetic
aaaaccgtgatggaatggaa

SEQ ID NO: 7 - DNA, 20 bp, Artificial Sequence
  synthetic
cacatgaagcagcacgactt

SEQ ID NO: 8 - DNA, 20 bp, Artificial Sequence
  synthetic
gaagttcaccttgatgccgt

SEQ ID NO: 9 - DNA, 20 bp, Artificial Sequence
  synthetic
cctagcagacaggcttgacc

SEQ ID NO: 10 - DNA, 20 bp, Artificial Sequence
  synthetic
ccatccagatctccagcact

SEQ ID NO: 11 - DNA, 4393 bp, adeno-associated virus 8
cagagagggagtggccaactccatcactaggggtagcgcgaagcgcctcccacgctgccgcgtcagcgctgacgtaaattacgtcataggggagtggtcctgtattagctgtcacgtgagtgcttttgcggcattttgcgacaccacgtggccatttgaggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgaactgggtggccgagaaggaatgggagctgcccccggattctgacatggatcggaatctgatcgagcaggcacccctgaccgtggccgagaagctgcagcgcgacttcctggtccaatggcgccgcgtgagtaaggccccggaggccctcttctttgttcagttcgagaagggcgagagctactttcacctgcacgttctggtcgagaccacgggggtcaagtccatggtgctaggccgcttcctgagtcagattcgggaaaagcttggtccagaccatctacccgcggggtcgagccccaccttgcccaactggttcgcggtgaccaaagacgcggtaatggcgccggcgggggggaacaaggtggtggacgagtgctacatccccaactacctcctgcccaagactcagcccgagctgcagtgggcgtggactaacatggaggagtatataagcgcgtgcttgaacctggccgagcgcaaacggctcgtggcgcagcacctgacccacgtcagccagacgcaggagcagaacaaggagaatctgaaccccaattctgacgcgcccgtgatcaggtcaaaaacctccgcgcgctatatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattacccagaaccgcatctaccgcatcctcgctctcaacggctacgaccctgcctacgccggctccgtctttctcggctgggctcagaaaaagttcgggaaacgcaacaccatctggctgtttggacccgccaccaccggcaagaccaacattgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagcctctccaggaccggatgtttaagttcgaactcacccgccgtctggagcacgactttggcaaggtgacaaagcaggaagtcaaagagttcttccgctgggccagtgatcacgtgaccgaggtggcgcatgagttttacgtcagaaagggcggagccagcaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacgtgcgagagaatgaatcagaatttcaacatttgcttcacacacggggtcagagactgctcagagtgtttccccggcgtgtcagaatctcaaccggtcgtcagaaagaggacgtatcggaaactctgtgcgattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcg

SEQ ID NO: 12 - DNA, 2045 bp, Mus musculus
ggaggaagaggaggaggcagcaattttaccacaagggacagccaagctggctttgcttctcttggccagcccaatgaccttcctcccatttgctgaccactcccccgggctggcctccctgctgctcgctcacataacagccagctggacagctctggggaggcaactcagaggctcttcctccggcctctagctgggtgctggcctgaacttcaccagagggaaagagctcttgggagagcctgacaggtgcagagaacctcagccatgttggccttcttgttctacggcttgctactggcggcctgtggctctgtcaccatgtcaaatccaggggagtccagcttcgacctagcagacaggcttgacccggttgagaagatagacaggcttgacctggttgagaagataggcgacacgcatgccaaagtgctggagatctggatggagctaggacgacgaagggaggtggatgctgccgagatgcatgcaatctgcagggtacaaccatcagccacgctgccaccggatcagccgcagatcaccggcttggttctcttccggcagctggggccgggctccaggcttgaggcctatttcagtctggagggcttcccagctgagcagaacgcctccaaccgtgccatccacgtgcatgagttcggggacctgagccagggctgcgattccaccgggccgcactacaacccgatggaggtgccgcaccctcagcacccgggcgactttggcaacttcgtggtgcgcaacggccagctctggaggcatcgcgtcggcctgaccgcgtcgctggccggaccgcacgccatcttgggccgctctgtggtggtccacgccggcgaggacgacctgggtaaaggtggcaaccaggccagcctgcagaacggcaatgcaggtcgccggctcgcctgctgcgtggtaggcaccagcagctccgccgcctgggagagccagacaaaggagcgcaagaagcggcggcgggagagcgagtgcaagaccacttaagcctcactcagggcctccgagccccgccgctgcacgcatagatgtctccaggcgcccccagacgcctctagtcaccccagaggcctctaggcgtcctagacagaggcctcccagacacctcagtcgcctctgcgcttccatgcacgccagacacctctgtatggcccccagatgcctccacgaacctccgcgcaccctagatgttctcccatgtcccggacaccgttcctctgtgtccaggacaccttagttaacccagaaatcttttcacgccctatgcacttccacagacccagatccttaatgctctagatccatcccgagcccctttgtgtcccaagacaatcccacaagcccctagtctttgagtctgctctcagagaaccccctcttcctccccagagatcgcatgtgctcagatactctcctcctctgaggacttcccagtgagcacctttgagagtactcccttggggtatactgaaatatcgcccaccccatttccttctgcccccttttgttttcttcctgtccccatagcacccgagactcctctcttccctagagacctcttttttcttccctttgttcctccgaggcgctctgggaccactctgacaccctcacccccacccccaagttccatgttcccgatcacctcctgcggaggccccaggttctgttttcatctgtttcccatatggtgcctgcaccccagggagagcagctccttagagagagtatttgggaacctttatgttgctcattaaaaacatagcaattcacaacacaatgcactggccttgtgtacttttttgagactttgcagcttagttttgttttgtttttgttttttttttcttcccgcccccaaaatatccctgagaatttgcaggtctcctcctctaatgaaagaagtttctatcattaattgctatgcctttttggaggactgaggacattaacaaggacgcttaaatgtgcatgtgtgtggcttctttacaaaaggacaccgacacagc

SEQ ID NO: 13 - protein, 251 aa, Mus musculus
MLAFLFYGLLLAACGSVTMSNPGESSFDLADRLDPVEKIDRLDLVEKIGDTHAKVLEIWMELGRRREVDAAEMHAICRVQPSATLPPDQPQITGLVLFRQLGPGSRLEAYFSLEGFPAEQNASNRAIHVHEFGDLSQGCDSTGPHYNPMEVPHPQHPGDFGNFVVRNGQLWRHRVGLTASLAGPHAILGRSVVVHAGEDDLGKGGNQASLQNGNAGRRLACCVVGTSSSAAWESQTKERKKRRRESECKTT

SEQ ID NO: 14 - DNA, 1546 bp, homo sapiens
ggggaggtctggcctgcttttcctccctgaactggcccaatgactggctccctcacgctgaccactcctctgggctggcctcctgcactcgcgctaacagcccaggctccagggacagcctgcgttcctgggctggctgggtgcagctctcttttcaggagagaaagctctcttggaggagctggaaaggtgcccgactccagccatgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcatgcagcggcgggacgacgacggcgcgctccacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggcgtgtgcgggcccgggctctgggagcgccaggcgcgggagcactcagagcgcaagaagcggcggcgcgagagcgagtgcaaggccgcctgagcgcggcccccacccggcggcggccagggacccccgaggcccccctctgcctttgagcttctcctctgctccaacagacaccctccactctgaggtctcaccttcgcctttgctgaagtctccccgcagccctctccacccagaggtctccctataccgagacccaccatccttccatcctgaggaccgccccaaccctcggagccccccactcagtaggtctgaaggcctccatttgtaccgaaacaccccgctcacgctgacagcctcctaggctccctgaggtacctttccacccagaccctccttccccaccccataagccctgagactcccgcctttgacctgacgatcttcccccttcccgccttcaggttcctcctaggcgctcagaggccgctctggggggttgcctcgagtccccccacccctccccacccaccaccgctcccgcggcaagccagcccgtgcaacggaagccaggccaactgccccgcgtcttcagctgtttcgcatccaccgccaccccactgagagctgctcctttgggggaatgtttggcaacctttgtgttacagattaaaaattcagcaattcagtaaaaaaaaaaaaaaaaaa

SEQ ID NO: 15 - DNA, 206 bp, Mus musculus
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggat

SEQ ID NO: 16 - DNA, 655 bp, homo sapiens
cctgagtttgaatctctccaactcagccagcctcagtttcccctccactcagtccctaggaggaaggggcgcccaagcgggtttctggggttagactgccctccattgcaattggtccttctcccggcctctgcttcctccagctcacagggtatctgctcctcctggagccacaccttggttccccgaggtgccgctgggactcgggtaggggtgagggcccaggggcgacagggggagccgagggccacaggaagggctggtggctgaaggagactcaggggccaggggacggtggcttctacgtgcttgggacgttcccagccaccgtcccatgttcccggcgggggccagctgtccccaccgccagcccaactcagcacttggttagggtatcagcttggtgggggcgtgagcccagccctggggcgctcagcccatacaaggccatggggctgggcgcaaagcatgcctgggttcagggtgggtatggtgccggagcagggaggtgagaggctcagctgccctccagaactcctccctggggacaacccctcccagccaatagcacagcctaggtccccctatataaggccacggctgctggcccttcctttgggtcagtgtcacctccaggatacagacagcccccctt

SEQ ID NO: 17 - DNA, 164 bp, Homo sapiens
gcggccaggccaggcggccggacaggtggggaggtctctgtggctctccacgcccccattggtctgaggaggactctatgccctttctgagcaggggcccagcctgggggaggccatttatacccctccccctgggcccaccagcccaactcgccgctgccggc

SEQ ID NO: 18 - DNA, 306 bp, Gallus gallus
ctggctggcttgtgtcagccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctgctctgcccgccccggggtgggcgccggggggaccttaaagcctctgccccccaaggagcccttcccagatagccgccggcacccaccgctccgtgggac

(19) United States
(12) Patent Application Publication
(10) Pub. No.: US 2013/0136729 A1
(43) Pub. Date: May 30, 2013

(54) COMPOSITIONS AND METHODS FOR TARGETING AND TREATING DISEASES AND INJURIES USING ADENO-ASSOCIATED VIRUS VECTORS

(71) Applicant: University of Virginia Patent Foundation, d/b/a Un, Charlottesville, VA (US)

(72) Inventors: Brent A. FRENCH, Charlottesville, VA (US); Brian H. ANNEX, Charlottesville, VA (US)

(73) Assignee: University of Virginia Patent Foundation, d/b/a University of Virginia Licensing & Ventures Group, Charlottesville, VA (US)

(21) Appl. No.: 13/673,351

(22) Filed: Nov. 9, 2012

Related U.S. Application Data
(60) Provisional application No. 61/558,716, filed on Nov. 11, 2011.

(51) Int. Cl. A61K 38/47; A61K 38/45; A61K 38/17

(52) U.S. Cl. CPC A61K 38/47; A61K 38/1719; A61K 38/45
USPC 424/94.61; 514/44 R; 514/44 A

(57) ABSTRACT

The present application discloses compositions and methods useful for targeting and treating injured or diseased muscle, including cardiac and skeletal muscle. Disclosed herein are adenoviral vectors modified to contain enhancers, promoters, and genes to target muscle with high efficiency and to induce tissue specific gene expression of transgenes.

### CROSS REFERENCE TO RELATED APPLICATIONS

This application is entitled to priority pursuant to 35 U.S.C. §119(e) to U.S. provisional patent application No. 61/558,716, filed on Nov. 11, 2011.

### STATEMENT REGARDING FEDERALLY SPONSORED RESEARCH OR DEVELOPMENT

This invention was made with government support under Grant Nos. R01 HL058582, R01 HL092305, and R01 HL101200, awarded by The National Institutes of Health. The government has certain rights in the invention.

### BACKGROUND

Myocardial ischemia/reperfusion (IR) injury often leads to progressive left ventricle (LV) remodeling and eventual heart failure. LV remodeling resulting from myocardial infarction involves expansion of the infarct zone, extension of cell death in the border zone, overall dilation of the LV chamber and ultimately heart failure. LV remodeling (as assessed by changes in LV end-systolic and end-diastolic volumes) is immediately apparent within the first day after myocardial infarction (MI) and continues for weeks in rodents and perhaps months in larger mammals. Therefore, early intervention is necessary to protect the heart against LV remodeling following ML particularly in small animal models where LV remodeling subsides within two weeks of reperfusion. Effective gene therapy interventions to prevent LV remodeling may therefore benefit from gene delivery systems that preferentially transduce cardiomyocytes at risk and provide a rapid onset of gene expression. Adenoviral vectors provide robust and rapid onset of gene expression in the myocardium following direct injection into the LV. However, the utility of adenoviral vectors is limited due to the immunological recognition of low-level adenoviral gene expression by the host, leading to the clearance of transfected cells. Furthermore, upon IV injection, adenoviral vectors accumulate primarily in the liver and have limited capacity to target the heart.

Adeno-associated viral (AAV) vectors provide for sustained, long-term gene expression in a wide variety of tissues and cause minimal immunological complications compared to other viral vectors being tested for gene therapy. In recent years, a variety of new AAV serotypes have been isolated that exhibit a wide range of tissue tropisms and provide for efficient transduction and long-term gene expression. In particular, serotypes AAV6, AAV8, and AAV9 transduce cardiomyocytes preferentially following systemic administration and provide uniform gene delivery throughout the myocardium. The most widely studied serotype, AAV2, has a prolonged lag phase of 4-6 weeks before reaching maximum gene expression in the heart. On the other hand, the more recently discovered AAV serotypes provide for an earlier onset of gene expression, approaching steady state levels within 2-3 weeks. However, the onset of gene expression provided by the newer serotypes of AAV still lags behind that achieved by adenoviral vectors. Thus, AAV2 vectors have typically been employed in preemptive gene therapy applications for Ml and LV remodeling, with the AAV vector being administered several weeks before the induction of ischemia/reperfusion injury. Recently, AAV2 was directly injected into the myocardium shortly after IR to evaluate the ability of therapeutic gene delivery to preserve cardiac function in a porcine model. These studies showed that, despite the expected lag phase before gene expression, direct injection of AAV2 vectors could modulate the LV remodeling process in large animals, and could help preserve LV function. Although these studies are encouraging, delivering therapeutic genes by systemic administration would offer greater clinical relevance. However, due to the delayed onset of gene expression from conventional AAV vectors in normal myocardium, there are no reports, to date, on the use of AAV vectors to deliver gene therapy to the heart by systemic administration after ischemia and reperfusion.

Peripheral arterial disease (PAD) is mainly caused by atherosclerosis, which results in obstructions in arterial beds other than the coronary arteries, and the most common site is the lower extremity where occlusive disease leads to impaired perfusion. PAD affects about 3-10% of adults in the world and 15-20% in those over 70 years. Many patients are not candidates for surgical or catheter based revascularization and while patients with PAD should be treated with medications that target atherosclerosis, medications like statins and angiotensin converting enzyme inhibitors have yet to prove effective at increasing blood flow to ischemic limbs. Gene therapy protocols for PAD using genes that, for example, encode angiogenic growth factors to augment collateral blood flow to ischemic tissues have been pursued for more than a decade. Results of clinical gene therapy studies for PAD, which to date have exclusively used plasmid and adenovirus-based vectors delivered intra-muscularly, or sometimes intravascularly, have been almost uniformly disappointing. Among the likely reasons for previous failures in human studies are the use of vectors that have short durations of expression and are inefficient at gene delivery when they are present in the target tissue, but perhaps no gap is greater than the fact that most of the ischemic muscle mass in a patient with PAD never receives gene therapy using the intra-muscular injection methods employed in clinical trials.

An ideal vector for skeletal muscle gene transfer would provide sustained gene expression, and could be administered with minimally invasive procedures without inducing any vector-related inflammatory responses in the host. Over the past decade, AAV vectors have emerged as arguably the single most promising gene delivery system for human gene therapy. Recombinant AAV vectors transduce a wide variety of tissues in vivo and provide for long-term gene expression without provoking significant immune responses. To date, over 100 AAV serotypes have been reported. A recent comparison of the more recently discovered serotypes showed that AAV9 transduction to heart, lung, and tibialis anterior muscle after intravenous (IV) injection is superior to all other serotypes and is age independent, whereas transduction to liver and kidney is age dependent.

The natural tissue tropism of the various AAV serotypes can be exploited to favor gene delivery to one organ over another. This tropism is based on the viral capsids recognizing specific viral receptors expressed on specific cell types, thus allowing a degree of cell specific targeting within a given organ. Cell-specific expression may be further aided by the use of tissue-specific promoters conferring gene expression restricted to a specific cell type. This is desirable for gene therapy applications targeting organ specific diseases, as this will help avoid any possible harmful side effects due to gene expression in off target organs. Recently, several muscle specific promoter constructs based on the muscle creatine kinase (MCK) regulatory region were shown to provide striated muscle-restricted gene expression. Of the several regulatory cassettes based on the MCK regulatory element, the CK6 promoter has been shown to provide skeletal muscle restricted gene expression with reduced expression in cardiac muscle 1. This is particularly desirable in the context of using AAV9 for PAD gene therapy via systemic administration since AAV9 has a known preference for cardiac over skeletal muscle. However, the use of skeletal muscle-specific promoters in combination with the more recent AAV serotypes in the context of PAD is largely unexplored and indeed the entire approach could, in theory, be limited by the fact that blood flow to the ischemic limb is reduced thus creating a barrier to intravascular gene delivery. Recently, cell surface N-linked glycans with terminal galactosyl residues were shown to serve as the primary receptor for AAV9. Desialylation of these galactosylated glycans was shown to markedly increase cell surface binding and transduction by AAV9.

There is a long felt need in the art for compositions and methods to treat muscle diseases and injury resulting from trauma or injuries such as infarction and the resulting ischemia and to better target muscle cells. The present invention satisfies these needs.

### SUMMARY OF THE INVENTION

The present invention relates to compositions and methods for targeting muscle with adeno-associated viral vectors comprising useful regulatory elements for achieving expression of genes of interest, and for preventing and treating injuries, diseases, and disorders of muscle. In one aspect, the injuries, disease, and disorders are associated with ischemia or are the result of ischemia. In one aspect, the vectors further comprise a gene of interest, which may be a therapeutic gene. The regulatory element may include an enhancer and/or a promoter. In one aspect, the enhancer and/or promoter are tissue specific for muscle, and may be specific for cardiac myocytes or for skeletal myocytes. The method is useful for treating various injuries, diseases, and disorders of muscle. The combination of specific AAV vectors, enhancers, promoters, and therapeutic genes, and fragments and homologs thereof that are used can be modified to ensure a high rate of targeting cells and tissues of interest and expression of therapeutic genes and genes of interest in the target cell of tissue of interest.

In one embodiment the muscle is cardiac muscle. In another embodiment, the muscle is skeletal muscle. In one aspect, the cardiac muscle is ventricular muscle. In one aspect, the vector preferentially targets ischemic regions of the muscle. In one aspect the ischemic region targeted is an infarct border zone. In one aspect, the method inhibits ventricular remodeling and heart failure associated with myocardial infarction and ischemia. In one aspect, the method inhibits peripheral artery disease associated with ischemia. In one aspect, the method is useful for preventing or treating an injury, disease, or disorder selected from the group consisting of myocardial infarction, reperfusion injury, heart failure, and peripheral artery disease.

In one aspect, the subject animal is a mammal. In one aspect, the mammal is a human. The compositions and methods of the invention can be used on many types of animals, including livestock, pets, birds, cats, dogs, reptiles, and amphibians, including animals in zoos.

It is disclosed herein that, inter alia, administration of recombinant AAV9 vector (SEQ ID NO:1) or vectors bearing the AAV9 capsid after ischemia and reperfusion provides preferential transduction to cardiomyocytes at risk in the infarct border zone, with the onset of gene expression occurring even earlier than that observed in normal myocardium, where the vector includes the other elements described herein. The AAV9 capsid sequence is described below. Further, it is disclosed that post-IR delivery by IV injection of an AAV9 vector carrying EcSOD protects the heart against subsequent LV remodeling. These findings have potential clinical relevance because they establish a precedent for the intravenous administration of AAV-mediated, cardiac-targeted gene therapy post-reperfusion to protect the heart against subsequent LV remodeling and ultimately heart failure. The present invention therefore encompasses not just left ventricular remodeling, but remodeling of the right ventricle as well. Injury and remodeling can occur in both ventricles. In one aspect, the capsid sequence component of SEQ ID NO:1 consists of the sequence of nucleotide residues from position 2116 to position 4329. One of ordinary skill in the art will appreciate that additional 5′ or 3′ nucleotides relative to 2116 and 4329 respectively may be used as long as the capsid function is maintained as desired.

Without wishing to be bound by any particular theory, it was hypothesized herein that ischemia induces desialylation of the cell surface glycans, resulting in increased availability of AAV9 receptors, and that this might suffice to overcome the barrier of reduced blood flow in ischemic tissues. Presently disclosed example 2 was performed to compare the magnitude and specificity of reporter gene expression driven by the human cytomegalovirus (CMV) immediate early and the minimal CK6 promoters packaged into AAV9 capsids and administered by IV injection in a mouse model of hindlimb ischemia (HLI). The wild-type AAV9 genome has the sequence of SEQ ID NOT, which encodes both the rep and cap genes. One of ordinary skill in the art will appreciate that elements of SEQ ID NO:1 can be used to prepare a recombinant AAV9 vector of the invention or that the cap sequence of SEQ ID NOT (nucleotide residues from position 2116 to position 4329) can be used to prepare the recombinant AAV9 vector of the invention (as disclosed herein). In one aspect, the AAV9 cap sequence can be used in combination with elements from other AAV serotypes. Using a novel approach that combines a muscle-specific promoter with an AAV serotype capsid that, preferentially transduces muscle, it is disclosed herein that targeted expression of reporter genes in ischemic muscles, particularly skeletal and cardiac muscle, following systemic delivery is not only possible, it is markedly enhanced relative to non-ischemic muscles and other tissues.

In one embodiment, the present invention encompasses the use of AAV8 and AAV8 capsids and other AAV serotype vectors and their capsids for targeting muscle. AAV8 has the sequence of SEQ ID NO:11.

In one embodiment of the invention, the AAV vector is tropic for the heart. In one aspect, it is tropic for cardiac myocytes.

In one embodiment, an AAV transduced gene is regulated by a tissue specific regulatory sequence or promoter inserted into the AAV vector.

The present application discloses multiple vectors, AAVs, and regulatory sequences useful in the vectors and AAVs to practice the methods of the invention. It is known in the art that some of the sequences can be modified without disrupting the desired activity.

AAV vectors of the invention may further comprise one or more promoters or enhancers or sequences encoding proteins, such as the cardiac Troponin-T (type 2) gene (multiple species; for example SEQ ID NOs:2 and 18), muscle creatine kinase gene (for example SEQ ID NOs:4, 15, 16 and the 365 bp proximal promoter region extending from the −358 to +7 nucleotide position relative to the transcription start site), the desmin promoter, or active fragments or modifications thereof.

In one aspect, the regulatory element of the recombinant AAV vector increases expression of the therapeutic gene in the targeted muscle. In one aspect, the regulatory element comprises at least one enhancer element and at least one promoter element. In one aspect, the regulatory element comprises at least one promoter element. In one aspect, the regulatory element comprises one enhancer element and one promoter element. In one aspect, the regulatory element is one promoter element.

In one aspect, a gene or therapeutic gene or sequence of the invention is a structural gene. A structural gene's transcription is under the control of a promoter, which is operably linked thereto.

In one aspect, a vector of the invention preferentially targets an infarct area. In one aspect, the infarct area is the infarct border zone. In one aspect, cardiomyocytes in an infarct border zone are preferentially targeted over similar cells not in the infarct border zone. In one aspect, a vector of the invention preferentially targets an ischemic area.

In one embodiment, the therapeutic gene used in the AAV vector is extracellular superoxide dismutase. In one aspect, it is extracellular superoxide dismutase 3 (SOD3 or EcSOD). In the heart, the progression of steps leading to heart failure are ischemia-reperfusion injury, myocardial infarction, ventricular remodeling, and then heart failure. The present invention provides for the use an AAV vector of the invention comprising a nucleotide sequence encoding an extracellular superoxide dismutase protein, which is effective in treating each step of the process. An AAV vector comprising a nucleotide sequence encoding an extracellular superoxide dismutase protein is also useful in skeletal muscle and for treating such diseases and disorders as PAD. In skeletal muscle, the progression of steps, potentially leading to loss of limb, are chronic ischemia, muscle necrosis, and then loss of limb. The AAV vectors disclosed and taught herein are useful for treating each step of the PAD process.

In one embodiment, extracellular superoxide dismutase protein is administered to the subject in addition to an AAV vector of the invention, including when the AAV vector comprises an EcSOD encoding sequence. In one aspect, the sequence encoding EcSOD is SEQ ID NO:12 or 14, or active homologs or fragments thereof. In one aspect, a useful vector for cardiac-selective gene expression is AcTnTEcSOD. In another aspect, a useful vector for skeletal muscle-selective expression is AcCK6EcSOD.

One of ordinary skill in the art will appreciate that depending on factors such as the age, sex, health, of the subject or the particular injury or disease being prevented or treated that the recombinant AAV vector can be administered in varying quantities, at different times, and various means. In one aspect, a recombinant AAV vector of the invention can be administered systemically, intravenously, by intracoronary infusion, locally, topically, or by direct injection into myocardium. In one aspect, the recombinant AAV vector is injected directly into the myocardium of a ventricle. In one aspect, the recombinant AAV vector is injected directly into a ventricle. In one aspect, the ventricle is a left ventricle.

In one embodiment, a subject is pretreated with an effective amount of neuraminidase or other desialylation agent to increase desialylation of cell surface N-linked glycarts. In one aspect, the pretreatment enhances AAV binding to its cognate receptor. In one aspect, the neuraminidase or other desialylation agent is applied systemically or locally.

In one embodiment, a recombinant AAV vector of the invention is useful for targeting muscle preferentially over other tissues. In one embodiment, a recombinant AAV vector of the invention is useful for increasing expression of a gene of interest preferentially in muscle. The compositions and methods disclosed herein encompass targeting and transducing muscle with an AAV vector. The method comprises administering to a subject a pharmaceutical composition comprising an effective amount of a recombinant adeno-associated viral (AAV) vector comprising a regulatory element. The regulatory element comprises at least one promoter element and optionally at least one enhancer element. An enhancer and promoter are operably linked. The recombinant AAV vector also may optionally comprise at least one gene operably linked to a promoter element. The AAV may comprise the entire AAV genome, or a homolog or fragment thereof, such as the capsid of the particular AAV. However, it should be noted that the entire AAV genome may not be useful in some situations because of a need to make the vector replication-deficient and/or to insert, genes of interest such as therapeutic genes.

The regulatory elements and the gene of interest may also be substituted with active fragments, modifications, or homologs thereof. In one aspect, the recombinant AAV vector preferentially targets skeletal muscle. In one aspect, the AAV is AAV8 (SEQ ID NO:11) or AAV9 (SEQ ID NO:1). In one embodiment, when targeting muscle, the subject is pretreated with an effective amount of neuraminidase or other desialylation agent to increase desialylation of cell surface N-linked glycans and enhance AAV binding to its cognate receptor. In one embodiment, the regulatory element is a 571 bp CK6 muscle creatine kinase enhancer/promoter regulatory element, and the 571 bp enhancer/promoter consists of the 206 bp sequence of SEQ ID NO:16 and the 365 bp proximal promoter region of the muscle creatine kinase genomic fragment having GenBank Accession No. AF188002, wherein the 365 bp proximal promoter region extends from nucleotide position −358 to +7 relative to the transcriptional start site. In one embodiment, at least one promoter comprises the sequence of SEQ ID NOs:4, 16, 17, or 18 or the 365 bp proximal promoter region of muscle creatine kinase extending from nucleotide position −358 to +7 relative to the transcriptional start site, an optional enhancer comprises the sequence of SEQ ID NO:15, and an optional gene or therapeutic gene comprises the sequence of SEQ ID NO:12 or 14.

A recombinant AAV vector can be prepared for use in knocking down specific genes in muscle with siRNA or miRNA expressed from an AAV vector of the invention. For example, an AAV9 vector has been prepared and used in combination with Examples 1-3 to knock-down transgenic eGFP gene expression in the heart (data not shown). In one aspect, when the AAV vector comprises a sequence encoding an siRNA or an miRNA of interest, the sequence of interest is inserted as the “gene of interest” in the vector. This method can be used in combination with use of a recombinant vector comprising a therapeutic gene, essentially doubling the power of the system, for example, by providing for the knock-down of disease-causing genes.

The present invention further provides a kit for administering a pharmaceutical composition comprising an AAV vector of the invention or for using an AAV vector of the invention, and an instructional material for the use thereof.

Sequences of the Inyention—

Summary of Sequences Used—

SEQ ID NO:1—AAV9 nucleic acid sequence; GenBank Accession No. AX753250.1, 4385 bp

SEQ ID NO:2—Gallus gallus troponin T type 2 (cardiac) (TNNT2) nucleic acid sequence (mRNA); GenBank Accession No. NM_{—}205449.1, 1185 bp (the whole gene has Gene ID: 396433)

SEQ ID NO:3—cardiac troponin T amino acid sequence encoded by nucleic acid sequence of SEQ ID NO:2

SEQ ID NO:4—Mus Musculus creatine kinase (Mck) gene, promoter region nucleic acid sequence; GenBank Accession No. AF188002, 3357 bp

SEQ ID NO:5—forward primer for amplifying luciferase

SEQ ID NO:6—reverse primer for amplifying luciferase

SEQ ID NO:7—eGFP forward primer

SEQ ID NO:8—eGFP reverse primer

SEQ ID NO:9—EcSOD forward primer

SEQ ID NO:10—EcSOD reverse primer

SEQ ID NO:11—Adeno-associated virus 8 nucleic acid sequence; GenBank Accession No. NC_{—}00626.1, 4393 bp

SEQ ID NO:12—Mus musculus superoxide dismutase 3, extracellular (Sod3), mRNA, GenBank Accession NM_{—}011435.3, 2045 bp

SEQ ID NO:13—Mus musculus superoxide dismutase 3, extracellular (Sod3) amino acid sequence (GenBank Accession No. NP 035565.1), 251 a.a., encoded by nucleic acid sequence SEQ ID NO:12.

SEQ ID NO:14—Human therapeutic cDNA 1: SOD3 (EC-SOD), GenBank Accession No. NM_{—}003102, 1546 bp (The protein for this cDNA has GenBank Accession No. NP 003093.2).

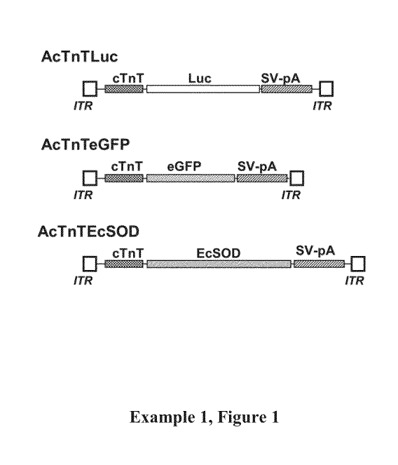

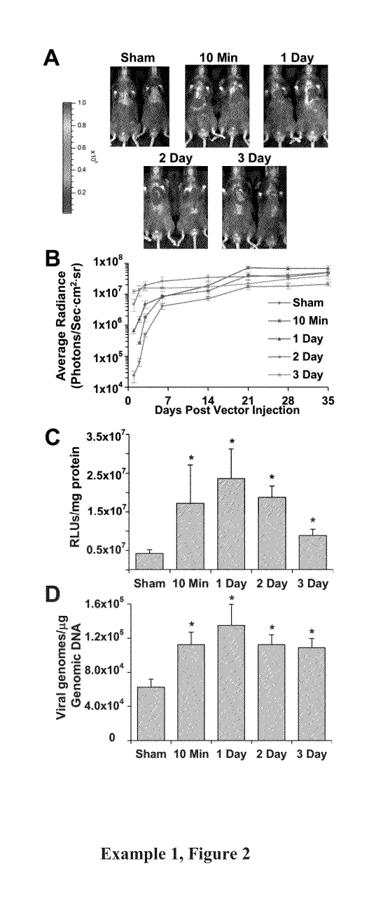

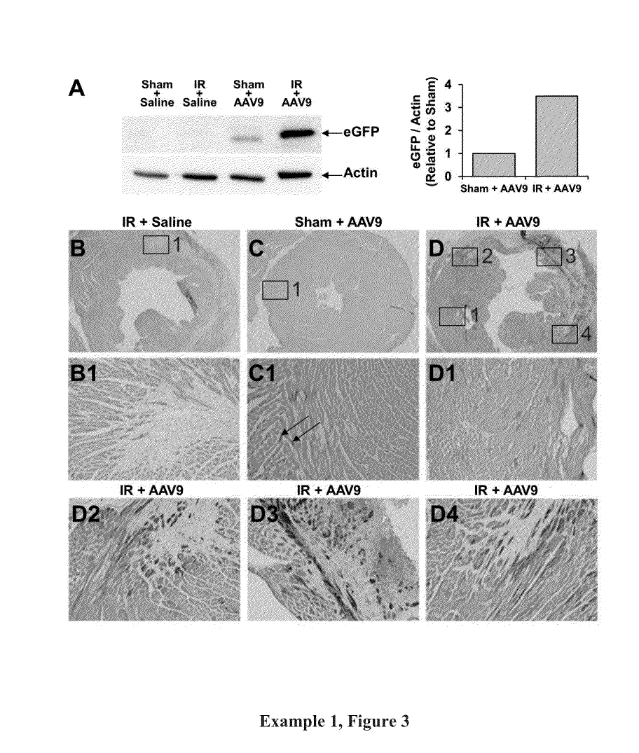

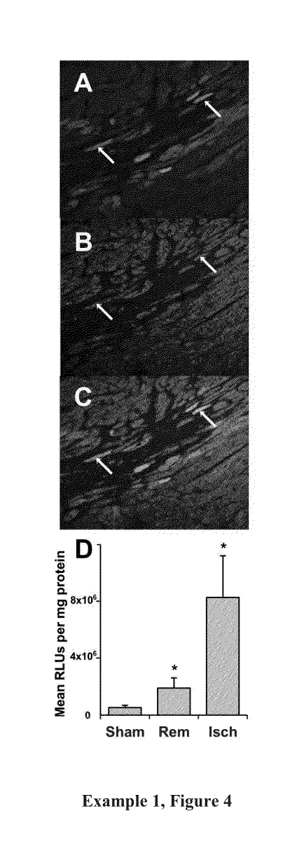

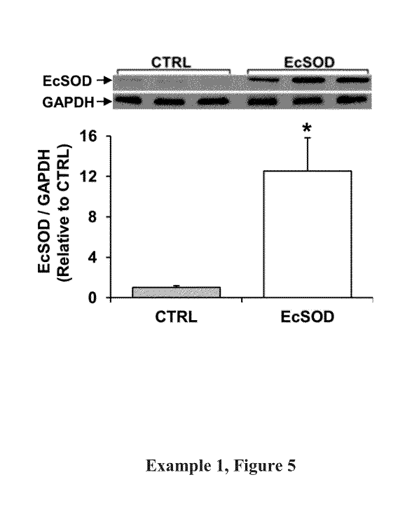

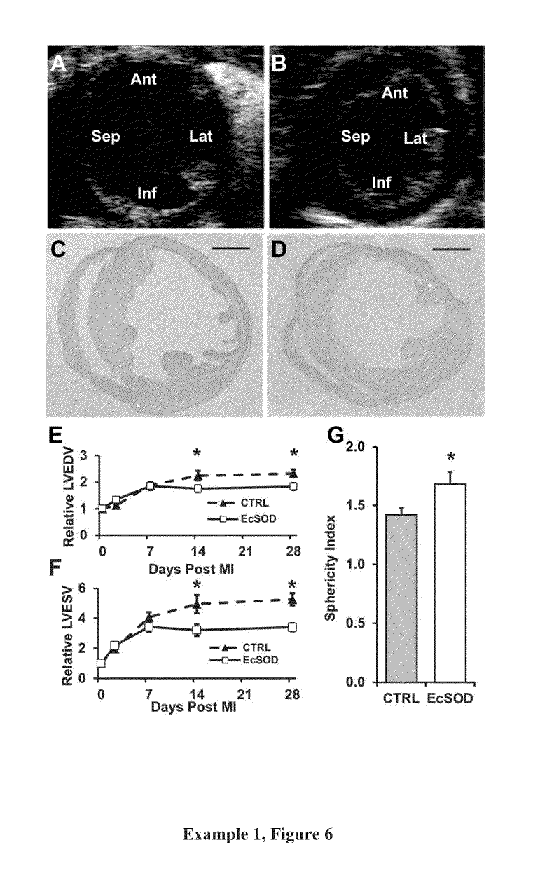

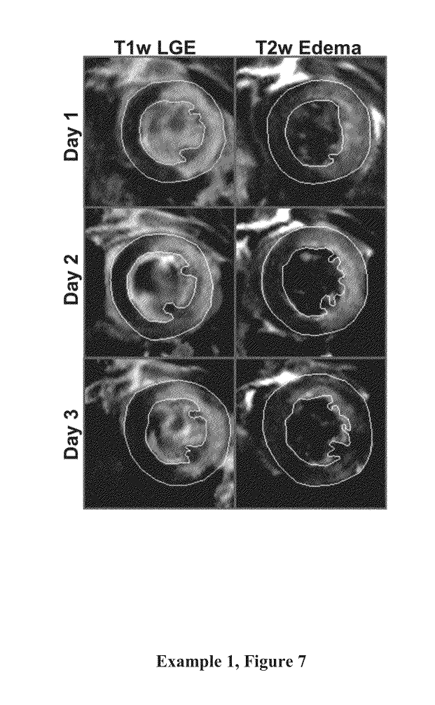

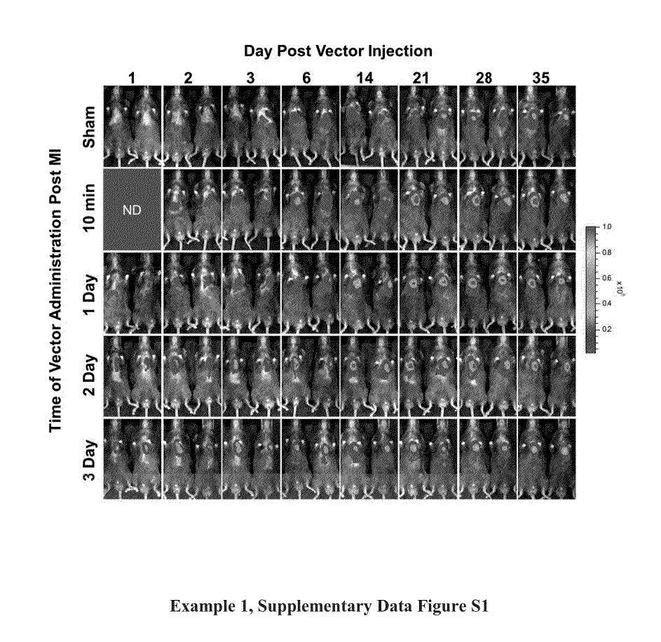

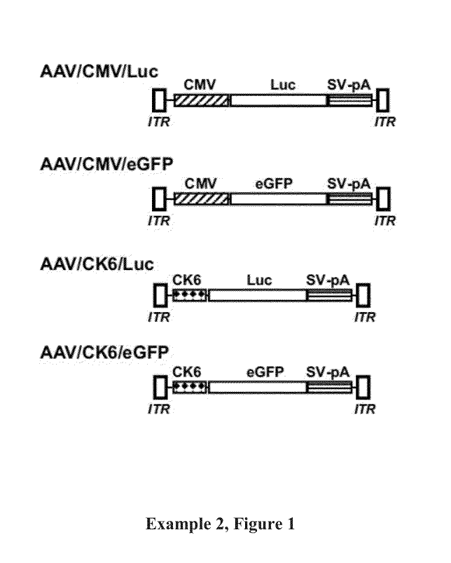

SEQ ID NO:15—206 bp fragment of SEQ ID NO:4 (depicted in Example 3, FIG. 1b which represents a murine muscle creatine kinase enhancer). It is also SEQ ID NO:20 of Souza et al., 2011, U.S. Pat. Pub. 2011/0212529.

SEQ ID NO:16—655 bp human MCK promoter sequence. It is also SEQ ID NO:18 of Souza et al., 2011 U.S. Pat. Pub. 2011/0212529.

SEQ ID NO:17—164 bp human fast skeletal muscle troponin I (TNNI2) promoter from Souza et al, US 2011/0212529, their SEQ ID NO:24

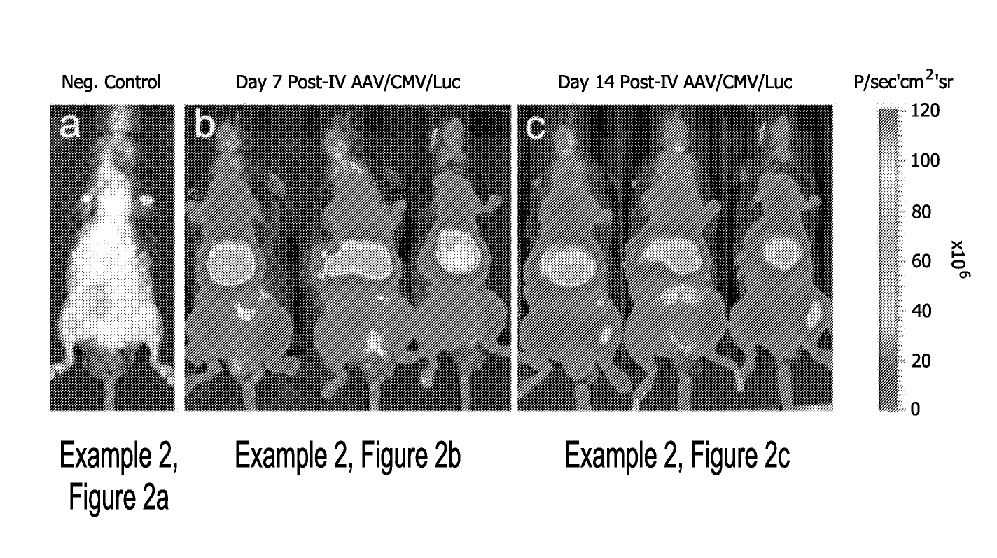

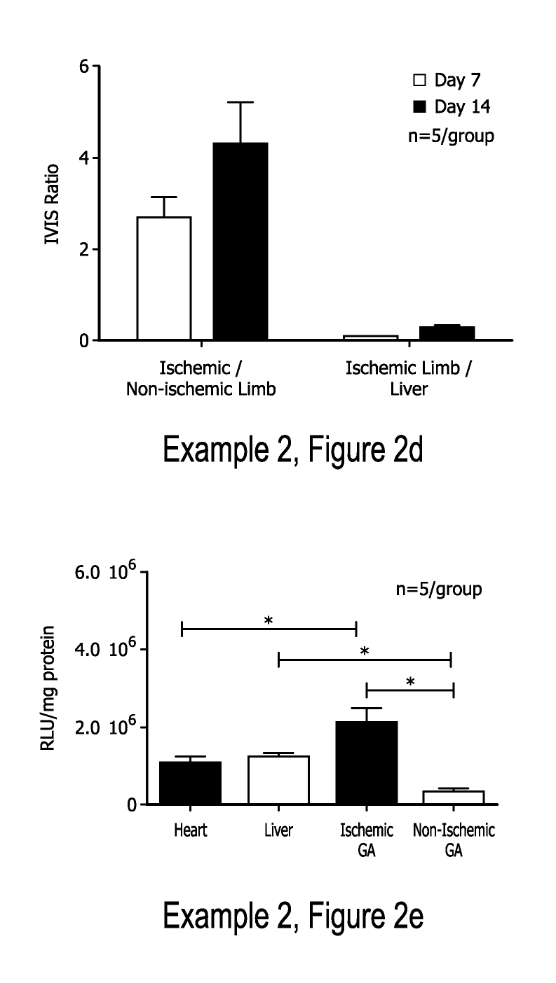

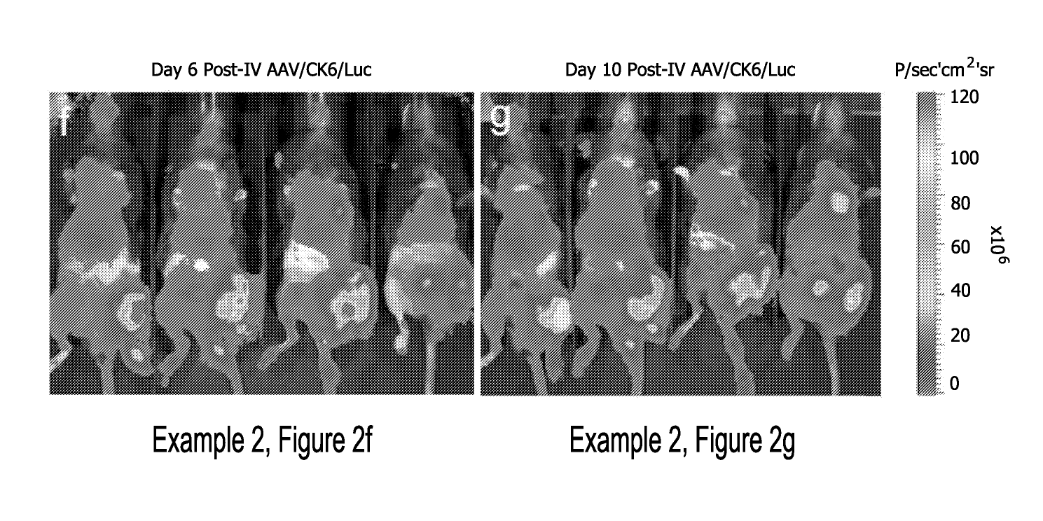

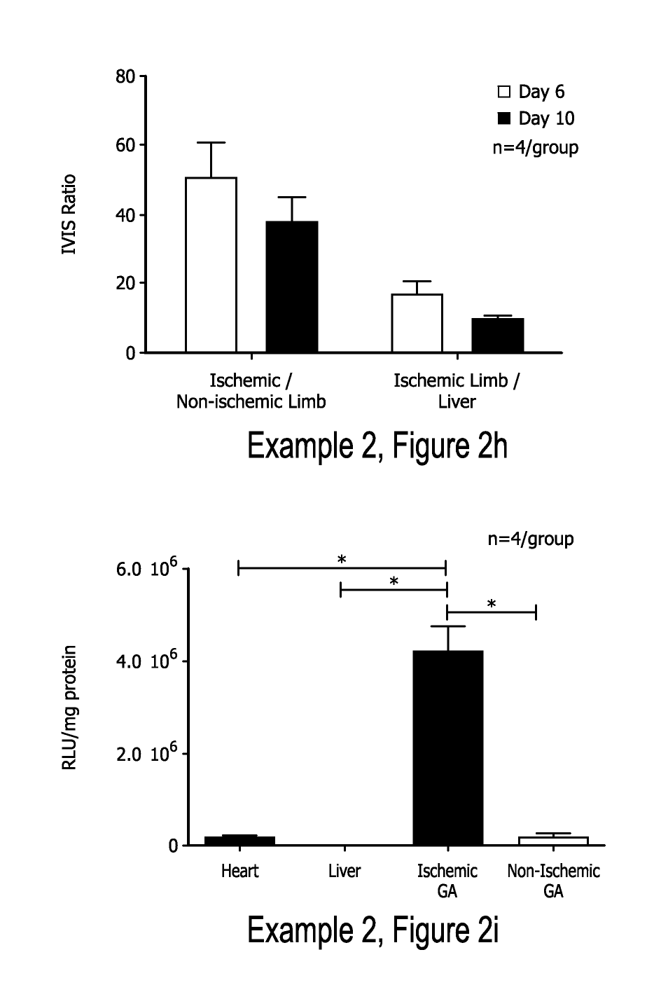

SEQ ID NO:18—306 bp chicken cardiac troponin-T 5′ region from −268 to +38 relative to the transcription start site (see FIG. 2 of U.S. Pat. No. 5,266,488)

Other useful sequences include the cap sequences of the useful AAV serotype vectors of the invention. For example, the cap sequence of AAV9 comprises nucleotide residues 2116-4329 of SEQ ID NO:1. Therefore, the invention encompasses the use of nucleotide residues 2116-4329 of SEQ ID NO:1 as the base for a recombinant AAV9 vector of the invention.

Sequences—

SEQ ID NO: 1
cagagagggagtggccaactccatcactaggggtaatcgcgaagcgcctcccacgctgccgcgtcagcgctgacgtagatt
acgtcataggggagtggtcctgtattagctgtcacgtgagtgcttttgcgacattttgcgacaccacatggccatttgaggtatat
atggccgagtgagcgagcaggatctccattttgaccgcgaaatttgaacgagcagcagccatgccgggcttctacgagattgt
gatcaaggtgccgagcgacctggacgagcacctgccgggcatttagactcttttgtgaactgggtggccgagaaggaatgg
gagctgcccccggattctgacatggatcggaatctgatcgagcaggcacccctgaccgtggccgagaagctgcagcgcga
cttcctggtccaatggcgccgcgtgagtaaggccccggaggccctcttctttgttcagttcgagaagggcgagagctactttca
cctgcacgttctggtcgagaccacgggggtcaagtccatggtgctaggccgcttcctgagtcagattcgggagaagctggtc
cagaccatctaccgcgggatcgagccgaccctgcccaactggttcgcggtgaccaagacgcgtaatggcgccggcgggg
ggaacaaggtggtggacgagtgctacatccccaactacctcctgcccaagactcagcccgagctgcagtgggcgtggacta
acatggaggagtatataagcgcgtgcttgaacctggccgagcgcaaacggctcgtggcgcagcacctgacccacgtcagcc
agacgcaggagcagaacaaggagaatctgaaccccaattctgacgcgcccgtgatcaggtcaaaaacctccgcgcgctac
atggagctggtcgggtggctggtggaccggggcatcacctccgagaagcagtggatccaggaggaccaggcctcgtacat
ctccttcaacgcctccaactcgcggtcccagatcaaggccgcgctggacaatgccggcaagatcatggcgctgaccaa
atccgcgcccgactacctggtaggcccttcacttccggtggacattacgcagaaccgcatctaccgcatcctgcagctcaacg
gctacgaccctgcctacgccggctccgtctttctcggctgggcacaaaagaagttcgggaaacgcaacaccatctggctgttt
gggccggccaccacgggaaagaccaacatcgcagaagccattgcccacgccgtgcccttctacggctgcgtcaactggac
caatgagaactttcccttcaacgattgcgtcgacaagatggtgatctggtgggaggagggcaagatgacggccaaggtcgtg
gagtccgccaaggccattctcggcggcagcaaggtgcgcgtggaccaaaagtgcaagtcgtccgcccagatcgaccccac
tcccgtgatcgtcacctccaacaccaacatgtgcgccgtgattgacgggaacagcaccaccttcgagcaccagcagcctctc
caggaccggatgtttaagttcgaactcacccgccgtctggagcacgactttggcaaggtgacaaagcaggaagtcaaagagt
tcttccgctgggccagtgatcacgtgaccgaggtggcgcatgagttttacgtcagaaagggcggagccagcaaaagacccg
cccccgatgacgcggataaaagcgagcccaagcgggcctgcccctcagtcgcggatccatcgacgtcagacgcggaagg
agctccggtggactttgccgacaggtaccaaaacaaatgttctcgtcacgcgggcatgcttcagatgctgcttccctgcaaaac
gtgcgagagaatgaatcagaatttcaacatttgcttcacacacggggtcagagactgctcagagtgtttccccggcgtgtcaga
atctcaaccggtcgtcagaaagaggacgtatcggaaactctgtgcgattcatcatctgctggggcgggctcccgagattgcttg
ctcggcctgcgatctggtcaacgtggacctggatgactgtgtttctgagcaataaatgacttaaaccaggtatggctgccgatg
gttatcttccagattggctcgaggacaacctctctgagggcattcgcgagtggtgggcgctgaaacctggagccccgaagcc
caaagccaaccagcaaaagcaggacgacggccggggtctggtgcttcctggctacaagtacctcggacccttcaacggact
cgacaagggggagcccgtcaacgcggcggacgcagcggccctcgagcacggcaaggcctacgaccagcagctgcagg
cgggtgacaatccgtacctgcggtataaccacgccgacgccgagtttcaggagcgtctgcaagaagatacgtcttttggggg
caacctcgggcgagcagtcttccaggccaagaagcgggttctcgaacctctcggtctggttgaggaaggcgctaagacggc
tcctggaaagaagagaccggtagagccatcaccccagcgttctccagactcctctacgggcatcggcaagaaaggccaaca
gcccgccagaaaaagactcaattttggtcagactggcgactcagagtcagttccagaccctcaacctctcggagaacctccag
cagcgccctctggtgtgggacctaatacaatggctgcaggcggtggcgcaccaatggcagacaataacgaaggcgccgac
ggagtgggtaattcctcgggaaattggcattgcgattccacatggctgggggacagagtcatcaccaccagcacccgaacct
gggcattgcccacctacaacaaccacctctacaagcaaatctccaatggaacatcgggaggaagcaccaacgacaacacct
actttggctacagcaccccctgggggtattttgacttcaacagattccactgccacttctcaccacgtgactggcagcgactcat
caacaacaactggggattccggccaaagagactcaacttcaagctgttcaacatccaggtcaaggaggttacgacgaacgaa
ggcaccaagaccatcgccaataaccttaccagcaccgtccaggtctttacggactcggagtaccagctaccgtacgtcctagg
ctctgcccaccaaggatgcctgccaccgtttcctgcagacgtcttcatggttcctcagtacggctacctgacgctcaacaatgg
aagtcaagcgttaggacgttcttctttctactgtctggaatacttcccttctcagatgctgagaaccggcaacaactttcagttcag
ctacactttcgaggacgtgcctttccacagcagctacgcacacagccagagtctagatcgactgatgaaccccctcatcgacc
agtacctatactacctggtcagaacacagacaactggaactgggggaactcaaactttggcattcagccaagcaggccctag
ctcaatggccaatcaggctagaaactgggtacccgggccttgctaccgtcagcagcgcgtctccacaaccaccaaccaaaat
aacaacagcaactttgcgtggacgggagctgctaaattcaagctgaacgggagagactcgctaatgaatcctggcgtggctat
ggcatcgcacaaagacgacgaggaccgcttctttccatcaagtggcgttctcatatttggcaagcaaggagccgggaacgat
ggagtcgactacagccaggtgctgattacagatgaggaagaaattaaagccaccaaccctgtagccacagaggaatacgga
gcagtggccatcaacaaccaggccgctaacacgcaggcgcaaactggacttgtgcataaccagggagttattcctggtatgg
tctggcagaaccgggacgtgtacctgcagggccctatttgggctaaaatacctcacacagatggcaactttcacccgtctcctc
tgatgggtggatttggactgaaacacccacctccacagattctaattaaaaatacaccagtgccggcagatcctcctcttaccttc
aatcaagccaagctgaactctttcatcacgcagtacagcacgggacaagtcagcgtggaaatcgagtgggagctgcagaaa
gaaaacagcaagcgctggaatccagagatccagtatacttcaaactactacaaatctacaaatgtggactttgctgtcaatacca
aaggtgtttactctgagcctcgccccattggtactcgttacctcacccgtaatttgtaattgcctgttaatcaataaaccggttaattc
gtttcagttgaactttggtctctgcg
SEQ ID NO: 2
ttcccagatagccgccggcacccaccgctccgtgggacctcggcacaggtagccaagcatgtcggactctgaagaggtcgt
tgaggaatacgagcaggagcaggaagaggagtatgtggaagaagaagaggaagaatggcttgaggaagacgacggtca
ggaggatcaggtagacgaggaggaagaggagacagaggaaaccacggcagaagaacaagaagatgaaacaaaagcac
caggagaaggtggtgagggggaccgggagcaggagcctggggaaggtgaatcaaagcccaaacccaagcccttcatgc
ccaacctggtgcctcccaaaatccctgatggcgagcgcctggatttcgatgacatccaccgcaagcgcatggagaaggacct
gaatgagctgcaggccctcatcgaagcccattttgagagcaggaagaaggaggaagaggagctcatctctctcaaggacag
gattgagcagcggagggcagagagggcagagcagcagcgcatccgcagcgagagggagaaggagcgccaggcccgc
atggctgaggagagagctcgcaaagaggaagaggaggcacggaagaaggctgagaaagaagctcggaaaaagaaagct
ttctccaacatgctgcactttggaggctacatgcagaagtcggagaagaagggtggcaagaagcaaacggagcgggagaa
gaagaaaaagatcctcagcgagcggcggaagcctctgaacatcgaccacctcagcgaagacaaactgagggacaaagcc
aaggagctgtggcaaaccatccgtgacctggaggctgagaaatttgacttgcaggagaagttcaagcggcagaagtacgag
atcaacgtccttcgaaatcgtgtcagtgaccaccagaaggtcaaagggtcaaaggctgcccgtgggaagaccatggtgggc
ggccggtggaagtagatggctctgaaggcaaaggtgaggctcagccatcagatgcagtgctgtgcgctcaacctatgccag
ggctctgctgcctccccaccatgcagtgcttgtacagtgcttgctgctggctccacgctgccggggtgggcaggtgctcagcg
aggcgctgattctcatctccacacccccacatgatgttgtgtctgtaaataaagagaggagtgaggggg
SEQ ID NO: 3
MSDSEEVVEEYEQEQEEEYVEEEEEEWLEEDDGQEDQVDEEEEETEETTAEEQEDE
TKAPGEGGEGDREQEPGEGESKPKPKPFMPNLVPPKIPDGERLDFDDIHRKRMEKD
LNELQALIEAHFESRKKEEELISLKDRIEQRRAERAEQQRIRSEREKERQARMAEER
ARKEEEEARKKAEKEARKKKAFSNMLHFGGYMQKSEKKGGKKQTEREKKKKILS
ERRKPLNIDHLSEDKLRDKAKELWQTIRDLEAEKFDLQEKFKRQKYEINVLRNRVS
DHQKVKGSKAARGKTMYGGRWK
SEQ ID NO: 4
ccatcctggtctatagagagagttccagaacagccagggctacagataaacccatctggaaaaacaaagttgaatgacccaagagg
ggttctcagagggtggcgtgtgctccctggcaagcctatgacatggccggggcctgcctactagcctctgaccctcagtggctccc
atgaactccttgcccaatggcatctttttcctgcgctccttgggttattccagtacccctcagcattccttcctcagggcctcgctcttctct
ctgctccctccttgcacagctggctagtccacctcagatgtcacagtgctctctcagaggaggaaggcaccatgtaccctctgtttcc
caggtaagggttcaatttttaaaaatggttttttgtttgtttgtttgtttgtttgtttgtttgtttttcaagacagggctcctctgtgtagtcctaact
gtcttgaaactccctctgtagaccaggtcgacctcgaactcttgaaacctgccacggaccacccagtcaggtatggaggtccctgga
atgagcgtcctcgaagctaggtgggtaagggttcggcggtgacaaacagaaacaaacacagaggcagtttgaatctgagtgtatttt
gcagctctcaagcaggggattttatacataaaaaaaaaaaaaaaaaaaaaaccaaacattacatctcttagaaactatatccaatgaaa
caatcacagataccaaccaaaaccattgggcagagtaaagcacaaaaatcatccaagcattacaactctgaaaccatgtattcagtga
atcacaaacagaacaggtaacatcattattaatataaatcaccaaaatataacaattctaaaaggatgtatccagtgggggctgtcgtcc
aaggctagtggcagatttccaggagcaggttagtaaatcttaaccactgaactaactctccagccccatggtcaattattatttagcatct
agtgcctaatttttttttataaatcttcactatgtaatttaaaactattttaattcttcctaattaaggctttctttaccatataccaaaattcacctc
caatgacacacgcgtagccatatgaaattttattgttgggaaaatttgtacctatcataatagttttgtaaatgatttaaaaagcaaagtgtt
agccgggcgtggtggcacacgcctttaatccctgcactcgggaggcaggggcaggaggatttctgagtttgaggccagcctggtct
acagagtgagttccaggacagccagggctacacagagaaaccctgtctcgaaccccccaccccccaaaaaaagcaaagtgttggt
ttccttggggataaagtcatgttagtggcccatctctaggcccatctcacccattattctcgcttaagatcttggcctaggctaccaggaa
catgtaaataagaaaaggaataagagaaaacaaaacagagagattgccatgagaactacggctcaatattttttctctccggcgaaga
gttccacaaccataccaggaggcctccacgttttgaggtcaatggcctcagtctgtggaacttgtcacacagatcttactggaggtgg
tgtggcagaaacccattccttttagtgtcttgggctaaaagtaaaaggcccagaggaggcctttgctcatctgaccatgctgacaagg
aacacgggtgccaggacagaggctggaccccaggaacaccttaaacacttcttcccttctccgccccctagagcaggctcccctca
ccagcctgggcagaaatgggggaagatggagtgaagccatactggctactccagaatcaacagagggagccgggggcaatact
ggagaagaggtctccccccaggggcaatcctggcacctcccaggcagaagaggaaacttccacagtgcatctcacttccatgaat
cccctcctcggactctgaggtccttggtcacagctgaggtgcaaaaggctcctgtcatattgtgtcctgctctggtctgccttccacagc
ttgggggccacctagcccacctctccctagggatgagagcagccactacgggtctaggctgcccatgtaaggaggcaaggcctgg
ggacacccgagatgcctggttataattaacccagacatgtggctgcccccccccccccaacacctgctgcctgagcctcaccccca
ccccggtgcctgggtcttaggctctgtacaccatggaggagaagctcgctctaaaaataaccctgtccctggtggatccagggtgag
gggcaggctgagggcggccacttccctcagccgcaggtttgttttcccaagaatggtttttctgcttctgtagcttttcctgcaattctgc
catggtggagcagcctgcactgggcttctgggagaaaccaaaccgggttctaacctttcagctacagttattgcctttcctgtagatgg
gcgactacagccccacccccacccccgtctcctgtatccttcctgggcctggggatcctaggctttcactggaaattccccccaggt
gctgtaggctagagtcacggctcccaagaacagtgcttgcctggcatgcatggttctgaacctccaactgcaaaaaatgacacatac
cttgacccttggaaggctgaggcagggggattgccatgagtgcaaagccagactgggtggcatagttagaccctgtctcaaaaaac
caaaaacaattaaataactaaagtcaggcaagtaatcctactcgggagactgaggcagagggattgttacatgtctgaggccagcct
ggactacatagggtttcaggctagccctgtctacagagtaaggccctatttcaaaaacacaaacaaaatggttctcccagctgctaatg
ctcaccaggcaatgaagcctggtgagcattagcaatgaaggcaatgaaggagggtgctggctacaatcaaggctgtgggggactg
agggcaggctgtaacaggcttgggggccagggcttatacgtgcctgggactcccaaagtattactgttccatgttcccggcgaagg
gccagctgtcccccgccagctagactcagcacttagtttaggaaccagtgagcaagtcagcccttggggcagcccatacaaggcca
tggggctgggcaagctgcacgcctgggtccggggtgggcacggtgcccgggcaacgagctgaaagctcatctgctctcaggggc
ccctccctggggacagcccctcctggctagtcacaccctgtaggctcctctatataacccaggggcacaggggctgcccccgggtc
ac
SEQ ID NO: 5
5′-AGAACTGCCTGCGTGAGATT-3′
SEQ ID NO: 6
5'-AAAACCGTGATGGAATGGAA-3′
SEQ ID NO: 7
5′-CACATGAAGCAGCACGACTT-3′
SEQ ID NO: 8
5′-GAAGTTCACCTTGATGCCGT-3′
SEQ ID NO: 9
5′-CCTAGCAGACAGGCTTGACC-3′
SEQ ID NO: 10
5′-CCATCCAGATCTCCAGCACT-3′
SEQ ID NO: 11
cagagagggagtggccaactccatcactaggggtagcgcgaagcgcctcccacgctgccgcgtcagcgctgacgtaaatta
cgtcataggggagtggtcctgtattagctgtcacgtgagtgcttttgcggcattttgcgacaccacgtggccatttgaggtatata
tggccgagtgagcgagcaggatctccattttgaccgcgaaatttgaacgagcagcagccatgccgggcttctacgagatcgt
gatcaaggtgccgagcgacctggacgagcacctgccgggcatttctgactcgtttgtgaactgggtggccgagaaggaatg
ggagctgcccccggattctgacatggatcggaatctgatcgagcaggcacccctgaccgtggccgagaagctgcagcgcg
acttcctggtccaatggcgccgcgtgagtaaggccccggaggccctcttctttgttcagttcgagaagggcgagagctactttc
acctgcacgttctggtcgagaccacgggggtcaagtccatggtgctaggccgcttcctgagtcagattcgggaaaagcttggt
ccagaccatctacccgcggggtcgagccccaccttgcccaactggttcgcggtgaccaaagacgcggtaatggcgccggc
gggggggaacaaggtggtggacgagtgctacatccccaactacctcctgcccaagactcagcccgagctgcagtgggcgt
ggactaacatggaggagtatataagcgcgtgcttgaacctggccgagcgcaaacggctcgtggcgcagcacctgacccac
gtcagccagacgcaggagcagaacaaggagaatctgaaccccaattctgacgcgcccgtgatcaggtcaaaaacctccgc
gcgctatatggagctggtcgggtggctggtggaccggggcatcacctccgagaagcagtggatccaggaggaccaggcct
cgtacatctccttcaacgccgcctccaactcgcggtcccagatcaaggccgcgctggacaatgccggcaagatcatggcgct
gaccaaatccgcgcccgactacctggtggggccctcgctgcccgcggacattacccagaaccgcatctaccgcatcctcgc
tctcaacggctacgaccctgcctacgccggctccgtctttctcggctgggccagaaaaagttcgggaaacgcaacaccatct
ggctgtttggacccgccaccaccggcaagaccaacattgcggaagccatcgcccacgccgtgcccttctacggctgcgtca
actggaccaatgagaactttcccttcaatgattgcgtcgacaagatggtgatctggtgggaggagggcaagatgacggccaa
ggtcgtggagtccgccaaggccattctcggcggcagcaaggtgcgcgtggaccaaaagtgcaagtcgtccgcccagatcg
accccacccccgtgatcgtcacctccaacaccaacatgtgcgccgtgattgacgggaacagcaccaccttcgagcaccagc
agcctctccaggaccggatgtttaagttcgaactcacccgccgtctggagcacgactttggcaaggtgacaaagcaggaagt
caaagagttcttccgctgggccagtgatcacgtgaccgaggtggcgcatgagttttacgtcagaaagggcggagccagcaa
aagacccgcccccgatgacgcggataaaagcgagcccaagcgggcctgcccctcagtcgcggatccatcgacgtcagac
gcggaaggagctccggtggactttgccgacaggtaccaaaacaaatgttctcgtcacgcgggcatgcttcagatgctgtttcc
ctgcaaaacgtgcgagagaatgaatcagaatttcaacatttgcttcacacacggggtcagagactgctcagagtgtttccccgg
cgtgtcagaatctcaaccggtcgtcagaaagaggacgtatcggaaactctgtgcgattcatcatctgctggggcgggctcccg
agattgcttgctcggcctgcgatctggtcaacgtggacctggatgactgtgtttctgagcaataaatgacttaaaccaggtatgg
ctgccgatggttatcttccagattggctcgaggacaacctctctgagggcattcgcgagtggtgggcgctgaaacctggagcc
ccgaagcccaaagccaaccagcaaaagcaggacgacggccggggtctggtgcttcctggctacaagtacctcggacccttc
aacggactcgacaagggggagcccgtcaacgcggcggacgcagcggccctcgagcacgacaaggcctacgaccagca
gctgcaggcgggtgacaatccgtacctgcggtataaccacgccgacgccgagtttcaggagcgtctgcaagaagatacgtct
tttgggggcaacctcgggcgagcagtcttccaggccaagaagcgggttctcgaacctctcggtctggttgaggaaggcgcta
agacggctcctggaaagaagagaccggtagagccatcaccccagcgttctccagactcctctacgggcatcggcaagaaag
gccaacagcccgccagaaaaagactcaattttggtcagactggcgactcagagtcagttccagaccctcaacctctcggaga
acctccagcagcgccctctggtgtgggacctaatacaatggctgcaggcggtggcgcaccaatggcagacaataacgaagg
cgccgacggagtgggtagttcctcgggaaattggcattgcgattccacatggctgggcgacagagtcatcaccaccagcacc
cgaacctgggccctgcccacctacaacaaccacctctacaagcaaatctccaacgggacatcgggaggagccaccaacga
caacacctacttcggctacagcaccccctgggggtattttgactttaacagattccactgccacttttcaccacgtgactggcag
cgactcatcaacaacaactggggattccggcccaagagactcagcttcaagctcttcaacatccaggtcaaggaggtcacgc
agaatgaaggcaccaagaccatcgccaataacctcaccagcaccatccaggtgtttacggactcggagtaccagctgccgta
cgttctcggctctgcccaccagggctgcctgcctccgttcccggcggacgtgttcatgattccccagtacggctacctaacact
caacaacggtagtcaggccgtgggacgctcctccttctactgcctggaatactttccttcgcagatgctgagaaccggcaaca
acttccagtttacttacaccttcgaggacgtgcctttccacagcagctacgcccacagccagagcttggaccggctgatgaatc
ctctgattgaccagtacctgtactacttgtctcggactcaaacaacaggaggcacggcaaatacgcagactctgggcttcagcc
aaggtgggcctaatacaatggccaatcaggcaaagaactggctgccaggaccctgttaccgccaacaacgcgtctcaacga
caaccgggcaaaacaacaatagcaactttgcctggactgagggaccaaataccatctgaatggaagaaattcattggctaat
cctggcatcgctatggcaacacacaaagacgacgaggagcgatttttcccagtaacgggatcctgatttttggcaaacaaaat
gctgccagagacaatgcggattacagcgatgtcatgctcaccagcgaggaagaaatcaaaaccactaaccctgtggctacag
aggaatacggtatcgtggcagataacttgcagcagcaaaacacggctcctcaaattggaactgtcaacagccagggggcctt
acccggtatggtctggcagaaccgggacgtgtacctgcagggtcccatctgggccaagattcctcacacggacggcaacttc
cacccgtctccgctgatgggcggctttggcctgaaacatcctccgcctcagatcctgatcaagaacacgcctgtacctgcgga
tcctccgaccaccttcaaccagtcaaagctgaactctttcatcacgcaatacagcaccggacaggtcagcgtggaaattgaatg
ggagctgcagaaggaaaacagcaagcgctggaaccccgagatccagtacacctccaactactacaaatctacaagtgtgg
actttgctgttaatacagaaggcgtgtactctgaaccccgccccattggcacccgttacctcacccgtaatctgtaattgcctgtta
atcaataaaccggttgattcgtttcagttgaactttggtctctgcg
SEQ ID NO: 12
ggaggaagaggaggaggcagcaattttaccacaagggacagccaagctggattgatctatggccagcccaatgaccttc
ctcccatttgctgaccactcccccgggctggcctccctgctgctcgctcacataacagccagctggacagctctggggaggca
actcagaggctcttcctccggcctctagctgggtgctggcctgaacttcaccagagggaaagagctcttgggagagcctgac
aggtgcagagaacctcagccatgttggccttcttgttctacggcttgctactggcggcctgtggctctgtcaccatgtcaaatcc
aggggagtccagcttcgacctagcagacaggcttgacccggttgagaagatagacaggcttgacctggttgagaagatagg
cgacacgcatgccaaagtgctggagatctggatggagctaggacgacgaagggaggtggatgctgccgagatgcatgcaa
tctgcagggtacaaccatcagccacgctgccaccggatcagccgcagatcaccggcttggttctcaccggcagctggggcc
gggctccaggcttgaggcctatttcagtctggagggcttcccagctgagcagaacgcctccaaccgtgccatccacgtgcatg
agttcggggacctgagccagggctgcgattccaccgggccgcactacaacccgatggaggtgccgcaccctcagcacccg
ggcgactttggcaacttcgtggtgcgcaacggccagctaggaggcatcgcgtcggcctgaccgcgtcgaggccggaccg
cacgccatcttgggccgctctgtggtggtccacgccggcgaggacgacctgggtaaaggtggcaaccaggccagcctgca
gaacggcaatgcaggtcgccggctcgcctgagcgtggtaggcaccagcagctccgccgcctgggagagccagacaaag
gagcgcaagaagcggcggcgggagagcgagtgcaagaccacttaagcctcactcagggcctccgagccccgccgctgc
acgcatagatgtaccaggcgcccccagacgcctctagtcaccccagaggcctctaggcgtcctagacagaggcctcccag
acacctcagtcgcctagcgcttccatgcacgccagacacctctgtatggcccccagatgcctccacgaacctccgcgcaccc
tagatgttacccatgtcccggacaccgttcctagtgtccaggacaccttagttaacccagaaatatttcacgccctatgcactt
ccacagacccagatccttaatgactagatccatcccgagcccattgtgtcccaagacaatcccacaagcccctagtattgag
tctgctctcagagaaccccctcttcctccccagagatcgcatgtgctcagatactacctcctctgaggacttcccagtgagcac
catgagagtactcccttggggtatactgaaatatcgcccaccccatttccttctgccccctatcttacttcctgtccccatagcac
ccgagactcactcttccctagagacctcttttttcttccctttgttcctccgaggcgctctgggaccactctgacaccctcacccc
cacccccaagttccatgttcccgatcacctcctgcggaggccccaggttctgttttcatctgtttcccatatggtgcctgcacccc
agggagagcagctccttagagagagtatttgggaacctttatgttgctcattaaaaacatagcaattcacaacacaatgcactgg
ccttgtgtacttttttgagactttgcagcttagttttgttttgtttttgttttttttttcttcccgcccccaaaatatccctgagaatttgcag
gtctcctcctctaatgaaagaagtttctatcattaattgctatgcctttttggaggactgaggacattaacaaggacgcttaaatgtg
catgtgtgtggcttctttacaaaaggacaccgacacagc
SEQ ID NO: 13
MLAFLFYGLLLAACGSVTMSNPGESSFDLADRLDPVEKIDRLDLVEKIGDTHAK
VLEIWMELGRRREVDAAEMHAICRVQPSATLPPDQPQITGLVLFRQLGPGSRLE
AYFSLEGFPAEQNASNRAIHVHEFGDLSQGCDSTGPHYNPMEVPHPQHPGDFG
NFVVRNGQLWRHRVGLTASLAGPHAILGRSVVVHAGEDDLGKGGNQASLQN
GNAGRRLACCVVGTSSSAAWESQTKERKKRRRESECKTT
SEQ ID NO: 14
ggggaggtctggcctgcttttcctccctgaactggcccaatgactggctccctcacgctgaccactcctctgggctggcctcct
gcactcgcgctaacagcccaggctccagggacagcctgcgttcctgggctggctgggtgcagctctcttttcaggagagaaa
gctctcttggaggagctggaaaggtgcccgactccagccatgctggcgctactgtgttcctgcctgctcctggcagccggtgc
ctcggacgcctggacgggcgaggactcggcggagcccaactctgactcggcggagtggatccgagacatgtacgccaag
gtcacggagatctggcaggaggtcatgcagcggcgggacgacgacggcgcgctccacgccgcctgccaggtgcagccgt
cggccacgctggacgccgcgcagccccgggtgaccggcgtcgtcctcttccggcagcttgcgccccgcgccaagctcgac
gccttcttcgccctggagggcttcccgaccgagccgaacagctccagccgcgccatccacgtgcaccagttcggggacctg
agccagggctgcgagtccaccgggccccactacaacccgctggccgtgccgcacccgcagcacccgggcgacttcggca
acttcgcggtccgcgacggcagcctctggaggtaccgcgccggcctggccgcctcgctcgcgggcccgcactccatcgtg
ggccgggccgtggtcgtccacgctggcgaggacgacctgggccgcggcggcaaccaggccagcgtggagaacgggaa
cgcgggccggcggctggcctgctgcgtggtgggcgtgtgcgggcccgggctctgggagcgccaggcgcgggagcactc
agagcgcaagaagcggcggcgcgagagcgagtgcaaggccgcctgagcgcggcccccacccggcggcggccaggga
cccccgaggcccccctctgcctttgagcttctcctctgctccaacagacaccctccactctgaggtctcaccttcgcctttgctga
agtctccccgcagccctctccacccagaggtctccctataccgagacccaccatccttccatcctgaggaccgccccaaccct
cggagccccccactcagtaggtctgaaggcctccatttgtaccgaaacaccccgctcacgctagacagcctcctaggctccctg
aggtacctttccacccagaccctccttccccaccccataagccctgagactcccgcctttgacctgacgatcttcccccttcccg
ccttcaggttcctcctaggcgctcagaggccgctctggggggttgcctcgagtccccccacccctccccacccaccaccgct
cccgcggcaagccagcccgtgcaacggaagccaggccaactgccccgcgtcttcagctgtttcgcatccaccgccacccca
ctgagagctgctcctttgggggaatgtttggcaacctttgtgttacagattaaaaattcagcaattcagtaaaaaaaaaaaaaaaa
aa
SEQ ID NO: 15
ccactacgggtctaggctgcccatgtaaggaggcaaggcctggggacacccgagatgcctggttataattaacccagacatg
tggagcccccccccccccaacacctgctgcctgagcctcacccccaccccggtgcctgggtcttaggctctgtacaccatgg
aggagaagctcgctctaaaaataaccctgtccctggtggat
SEQ ID NO: 16
cctgagtttgaatctctccaactcagccagcctcagtttcccctccactcagtccctaggaggaaggggcgcccaagcgggttt
ctggggttagactgccctccattgcaattggtccttctcccggcctctgcttcctccagctcacagggtatctgctcctcctggag
ccacaccttggttccccgaggtgccgctgggactcgggtaggggtgagggcccaggggcgacagggggagccgagggc
cacaggaagggctggtggctgaaggagactcaggggccaggggacggtggcttctacgtgcttgggacgttcccagccac
cgtcccatgttcccggcgggggccagctgtccccaccgccagcccaactcagcacttggttagggtatcagcttggtggggg
cgtgagcccagccctggggcgctcagcccatacaaggccatggggctgggcgcaaagcatgcctgggttcagggtgggta
tggtgccggagcagggaggtgagaggctcagctgccctccagaactcctccctggggacaacccctcccagccaatagca
cagcctaggtccccctatataaggccacggctgctggcccttcctttgggtcagtgtcacctccaggatacagacagcccccct
t
SEQ ID NO: 17
gcggccaggccaggcggccggacaggtggggaggtctctgtggctctccacgcccccatt
ggtctgaggaggactctatgccctttctgagcaggggcccagcctgggggaggccattta
tacccctccccctgggcccaccagcccaactcgccgctgccggc
SEQ ID NO: 18
ctggctggcttgtgtcagccctcgggcactcacgtatctccgtccgacgggtttaaaatagcaaaactctgaggccacacaata
gcttgggcttatatgggctcctgtgggggaagggggagcacggagggggccggggccgctgctgccaaaatagcagctca
caagtgttgcattcctctctgggcgccgggcacattcctgctgctctgcccgccccggggtgggcgccggggggaccttaaa
gcctctgccccccaaggagcccttcccagatagccgccggcacccaccgctccgtgggac

Various aspects and embodiments of the invention are described in further detail below,

### BRIEF DESCRIPTION OF THE DRAWINGS

Example 1, FIG. 1. Schematic representation of AAV vectors: AcTnTLuc, AcTnTeGFP and AcTnTEcSOD carry the firefly luciferase, eGFP and EcSOD cDNA, respectively, driven by the cardiac troponin-T (cTnT) promoter. AAV inverted terminal repeats (ITR) and SV40 poly-adenylation sites (SV-pA) are indicated.

Example 1, FIG. 2. Experiments characterizing the effects of delaying the intravenous injection of AAV9 for various periods of time after reperfusion. Sham-operated mice (Sham) or mice that underwent 30 min IR (n==4 per group) were injected intravenously with AAV9 carrying AcTnTLuc, 1×10¹¹ viral genomes/mouse at the indicated times (10 min, 1 day, 2 days or 3 days) after reperfusion. (A) Representative bioluminescence images acquired at 2 days following vector administration for two mice from each group. Additional bioluminescence images (of mice obtained at days 1, 2, 3, 6, 14, 21, 28, and 35 after vector administration) are shown in Supplementary Data (Fig. S1). (B) Graph showing the time course of luciferase expression in mice. For each group of mice, the mean values of bioluminescence as average radiance (photons/s·cm²·sr) were obtained from the regions of interest and plotted against time after AcTnTLuc vector injection. (C) Quantitative determination of luciferase activity in protein extracts from hearts collected 35 days after vector administration. Luciferase activities are expressed as relative light units per mg tissue (RLUs/mg tissue, *p<0.05 vs. sham). (D) AAV vector genome copy numbers in hearts following systemic administration of AcTnTeGFP (1×10¹¹ viral genomes/mouse) in sham-operated mice (sham) or at the indicated times (10 min, 1 day, 2 days, or 3 days) after reperfusion. Results are expressed as viral genomes/μg genomic DNA (*p<0.05 vs. sham).

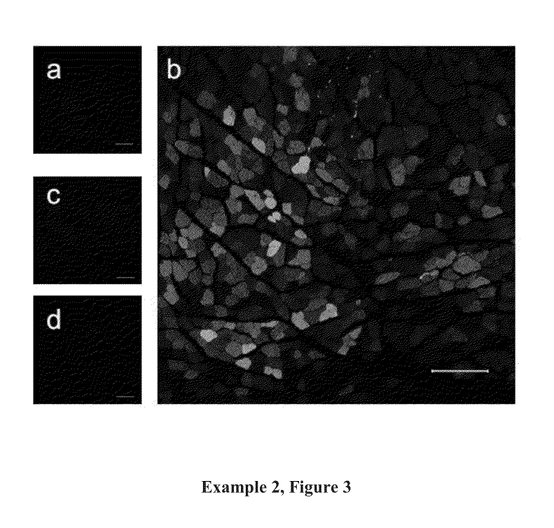

Example 1, FIG. 3. Distribution of transgene expression within the heart as a result of AAV administered after IR injury: (A) Western blot showing the expression of eGFP in mouse hearts. Sham-operated mice (Sham) or mice that underwent 30 min of ischemia (IR) were injected with saline or AcTnTeGFP (AAV9) at 10 min following reperfusion. Five days later, protein extracts were prepared from the mouse hearts and eGFP expression was detected by immunoblot analysis. To control for sample-loading error, the blot was then stripped and re-probed with an antibody against actin. The fold-induction due to ischemia is graphed at right relative to the sham-operated control. Panels (B)-(D) show immunohistochemistry for eGFP performed in short-axis myocardial tissue sections from: (B) mice that underwent 30 min of coronary artery occlusion, but were injected IV with vehicle (IR+saline), (C) sham-operated mice that were injected with AcTnTeGFP (Sham+AAV9), and (D) mice that underwent 30 min of coronary artery occlusion and were injected with AcTnTeGFP 10 min after reperfusion (IR+AAV9). Five days following vector administration, eGFP expression in the mouse hearts was evaluated by immunohistochemistry on slides counterstained with eosin. Panels (B)-(D) show photomicrographs taken at 4× magnification. Panels B1, C1, D1, D2, D3, and D4 show 40× magnifications of the areas indicated by rectangles in Panels B, C, and D, respectively. Note that combination of the red eosin counterstain and the brown DAB chromogen (labeling the antibody against eGFP) produce a reddish-brown color in eGFP-positive cells.

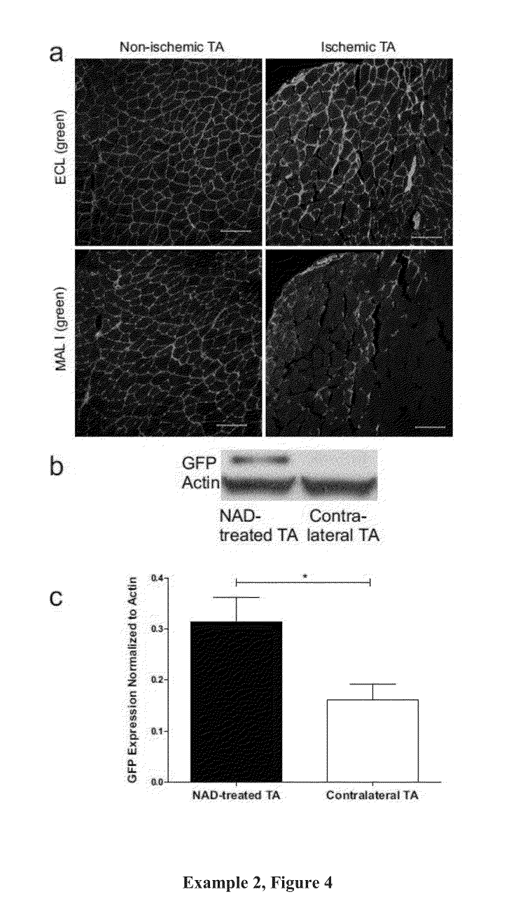

Example 1, FIG. 4, Post-MI AAV9 administration results in gene expression primarily localized to cardiomyocytes bordering the edge of the infarct. Mice were injected with AcTnTeGFP or AcTnTLuc at 10 min after IR injury. Five days later, (A) eGFP expression was detected by fluorescence microscopy (green). (B) Myoglobin expression in the same section was detected using an antibody against myoglobin (red). Note that the infarct region in the center of the field is evidenced by the loss of myosin (i.e., lack of red staining). (C) An overlay of panels A and B indicates in yellow to green the location of cardiomyocytes expressing various levels of eGFP (white arrows indicate two examples). (D) Hearts from mice injected with AcTnTLuc were collected 5 days following vector administration for quantitative luciferase activity assays. The graph shows the results of quantitative luciferase activity assays on protein extracts from hearts of sham-operated mice (Sham) or mice that underwent 30 min of coronary occlusion. Remote (Rem) and ischemic (Isch) regions of hearts from mice that underwent 30 min of coronary occlusion prior to AAV9 injection were separated under a dissecting microscope for luciferase activity assay. Hearts were collected 5 days following vector administration for quantitative luciferase activity assays. Luciferase activities are expressed RLUs/mg protein (*p<0.05 vs. sham, **p<0.05 vs. sham or remote).

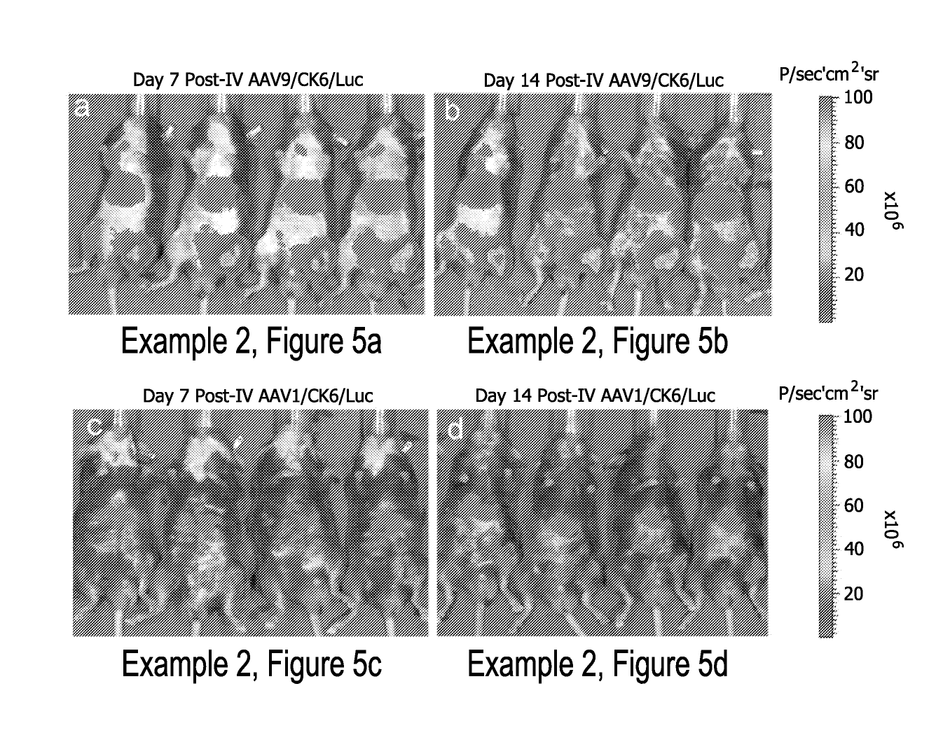

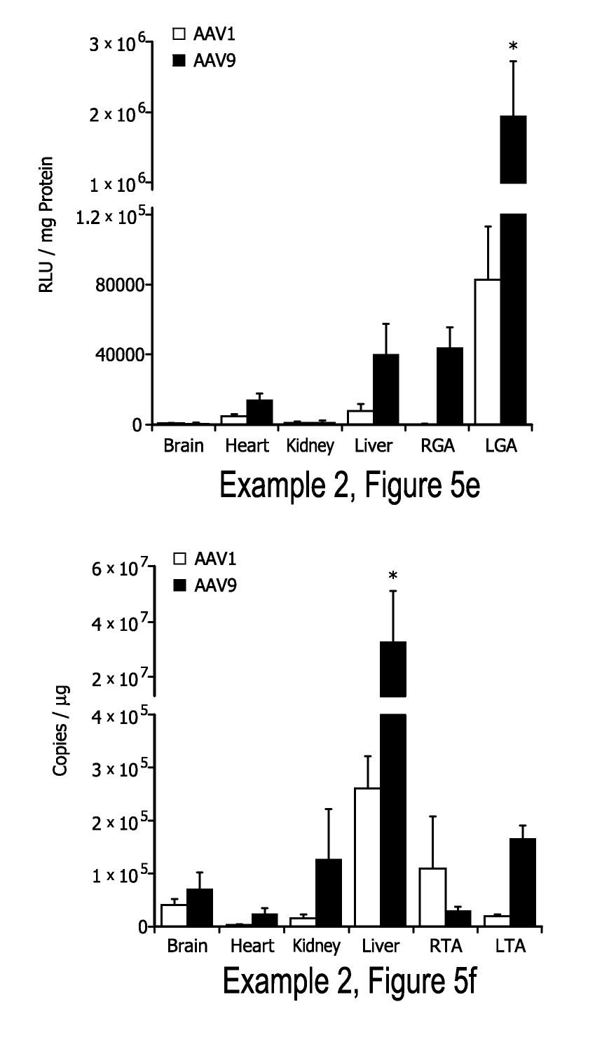

Example 1, FIG. 5. AAV9 carrying EcSOD administered after IR injury provides a 1.2.5-fold increase in EcSOD expression: Ten minutes after a 60 minute IR injury, mice were either injected IV with AcTnTEcSOD (EcSOD, n=8) or were used as controls (CTRL, n=9). Hearts were collected on day 29 (one day following the last echocardiography session described below in FIG. 6) and Western blot analysis performed on 3 hearts from each group revealed a 12.5-fold increase in GAPDH-normalized EcSOD expression in the EcSOD-treated group compared to the control group (p<0.05).

Example 1, FIG. 6. AAV9 carrying EcSOD administered after IR injury attenuates LV remodeling: Ten minutes after a 60 minute IR injury, mice were either injected IV with AcTnTEcSOD (EcSOD, n=8) or were used as controls (CTRL, n=9). Representative short-axis echo images of mouse hearts at end-systole from the control (A) and EcSOD-treated (B) groups at Day 28 post-MI. For orientation, images are labeled on the anterior (Ant), inferior (Inf), septal (Sep), and lateral (Lat) walls. Note that end-systolic chamber volume is appreciably smaller, and that the ventricular walls are appreciably thicker in the EcSOD-treated heart. (B) as compared to the control heart (A). One day after the final echocardiography session, hearts were explanted for histochemical staining. Representative paraffin-embedded sections from control and EcSOD-treated groups were stained with H&E (C and D, respectively). Scale bars=1 mm. LV remodeling was measured by LV end-diastolic (E) and LV end-systolic (F) volumes. Measurements were obtained by high-resolution echocardiography performed at baseline (1 day before MI surgery) and at days 2, 7, 14 and 28 after MI. Results are reported as percent increase over baseline (*p<0.05 vs. CTRL on same day). Sphericity index (G) was also significantly improved in the EcSOD-treated group on Day 28 compared to the infarcted control group (CTRL, *p<0.05 vs. CTRL).

Example 1, FIG. 7, Cardiac MRI demonstrates that the infarct and surrounding border zone are edematous after IR. Myocardial infarct area and edematous regions were detected by T1w LGE and T2w CMR imaging (left and right columns), respectively. Representative short-axis images acquired at the mid-left ventricular level from the same mouse heart at 1, 2 and 3 days post-IR are shown. The infarct regions enhanced by T1w LGE lie within the areas at risk represented by the enhanced areas in the T2w images, demonstrating that edema exists within both the infarct and infarct border zone.

Example 1. Fig. S1. Bioluminescence images showing the time course of luciferase expression in sham-operated mice and in mice after myocardial infarction (MI): Ischemia was induced by a 30 min. occlusion of the descending coronary artery followed by reperfusion. Mice (n=4) per group were injected with 1×10¹¹ viral genomes/mouse via the jugular vein at the indicated time (10 min, 1 day, 2 day or 3 day) after reperfusion. Bioluminescence images of mice were acquired at days 1, 2, 3, 6, 14, 21, 28, and 35 after vector administration for each group. ND=not determined.

Example 2, FIG. 1. Summary of AAV vectors: AAV/CM V/Luc and AAV/CK6/Luc cany the firefly luciferase gene (Luc) driven by CMV and CK6 promoters, respectively. AAV/CMV/eGFP and AAV/CK6/eGFP carry the eGFP gene driven by CMV and CK6 promoters, respectively. AAV inverted terminal repeats (ITR), and SV40 polyadenylation sites (SV-pA) are also indicated.

Example 2, FIG. 2. Time course and tissue distribution of CMV- and CK6-mediated luciferase expression from AAV-9 following intravenous (IV) injection 7-8 days after hindlimb ischemia (HLI) surgery, (a) Negative control consisting of an age-matched C57Bl/6 male mouse that did not undergo HLI and did not receive any vector. In the CMV group, b and c, adult C57Bl/6 mice (n=5 per group) were injected with 4.15×10¹¹ viral genomes/mouse via jugular vein. In vivo bioluminescence (IVIS) images were obtained on the 7th day (b) and 14th day (c) following vector administration. In the CK6 group, f and g, adult C57Bl/6 mice (n=4 per group) were injected with 4.15×10¹¹ viral genomes/mouse via jugular vein. In vivo bioluminescence (IVIS) images obtained on the 6th day (f) and 10th day (g) following vector administration, (d, h) For each group of mice, the mean values of bioluminescence as average radiance (photons/s·cm2·sr) were obtained from the regions of interest in Panels b, c, f and g and plotted as ratios of Ischemic limb to Non-ischemic limb and Ischemic limb to upper abdomen (corresponding to liver), (e, i) Bar graph showing luciferase activities in tissue extracts in the CMV and CK6 groups, respectively. Protein extracts from various tissues were collected 10-14 days after vector administration for homogenization and in vitro luciferase assays. Luciferase activities are expressed as relative light units per mg protein (RLU/mg protein) (Mean±SEM, *p<0.05).

Example 2, FIG. 3. Fluorescence microscopy of muscle cryosections from mice injected IV with AAV-9 vectors carrying CMV or CK6 promoters. The AAV vectors AAV/CK6/eGFP and AAV/CMV/eGFP were packaged into AAV-9 capsids. Adult C57Bl/6 mice were injected with 4.15×10¹¹ viral genomes/mouse (n=2 for the AAV/CK6/eGFP group and 5 for the AAV/CMV/eGFP group) via jugular vein. Two weeks following vector administration, 15 μm cryosections of the tibialis anterior (TA) muscles from ischemic and non-ischemic hindlimbs were prepared for analysis by confocal microscopy. All images shown here were captured at 5× magnification with a constant 0.5 sec exposure. Panels a (non-ischemic) and b (ischemic) show TA muscles from mice injected with AAV/CK6/eGFP, while panels c (non-ischemic) and d (ischemic) represent those injected with AAV/CMV/eGFP. Bar=200 μm.

Example 2, FIG. 4. Differential distribution of sialylated and desialylated cell surface glycans in ischemic and non-ischemic muscle, and quantitation of CK6-mediated eGFP expression from AAV-9 following intravenous (IV) injection in mice pre-treated with intramuscular (IM) injection of neuraminidase, (a) Fluorescence microscopy demonstrating the distribution of sialylated and desialylated cell surface glycans in ischemic and non-ischemic TA muscles of adult BALB/c mice (n=3) on the 7th day after HLI surgery. Representative fluorescent photomicrographs of the ischemic (I) and non-ischemic (NI) TA sections stained with ECL (green, upper row), MAL I (green, lower row) and actin (red). Bar=200 μm. (b) Adult C57Bl/6 mice (n=9) were pre-treated with 1M neuraminidase in the left TA muscles and 2-4 h later, given 4.15×10¹¹ viral genomes/mouse of AAV.MCK6.eGFP.bGH via jugular vein. 14 days following vector administration, eGFP expression was detected by Western blot analysis. Levels of eGFP expression were normalized to actin protein. Contralateral TA muscles served as negative control, (c) Bar graph showing the quantification of eGFP expression by Western blot analysis (Mean±SEM, *p<0.05).

Example 2, FIG. 5. Time course of CK6-mediated luciferase expression and tissue distribution of vector genomes from AAV-9 and AAV-1 following intravenous (IV) injection 7-8 days after hindlimb ischemia (HLI) surgery. Adult C57Bl/6 mice (n=5 per group) were injected with 4.15×10¹¹ viral genomes/mouse via jugular vein. In vivo bioluminescence (IVIS) images were obtained on the 7th day (a for AAV-9 and c for AAV-1 groups) and 14th day (b for AAV-9 and d for AAV-1 groups) following vector administration. Bar graph showing luciferase activities (e) and vector genome copy numbers (f) in tissue extracts from the AAV-9 and AAV-1 groups. Protein extracts from various tissues were collected 14-16 days after vector administration for homogenization and in vitro luciferase assays. Luciferase activities are expressed as relative light units per rag protein (RLU/mg protein). Genomic DNA was isolated from each of the indicated tissues and used to determine vector genome copy numbers per μg host genomic DNA (mean±SEM, *p<0.05).

Example 3, FIG. 1. Muscle creatine kinase (MCK) promoter and enhancer reproduced from Hauser et al., 2000. (a) The sequence of a 3355-bp genomic fragment of the murine MCK transcriptional regulatory region extending from −3348 to +7 relative to the transcriptional start site has been deposited in the GenBank database (Accession No. AF188002; also provided as SEQ ID NO:4 herein), and the corresponding restriction map is shown, (b) The sequence (SEQ ID NO:15) of the 206-bp MCK upstream enhancer is shown, with protein binding sites underlined. The sequence alterations corresponding to the 2R and S5 modifications are indicated above the wild-type sequence. The NcoI site indicated marks the upstream boundary of the MEF2 deletion in construct CK5.

Example 3, FIG. 2. Transcriptional regulatory cassettes based on the muscle creatine kinase promoter and enhancer (reproduced from Hauser et al., 2000). CK3 contains the full 3355-bp region extending from −3348 to +7 relative to the transcriptional start site. CK2 extends from −1256 to +7. CK5 contains part of the 2RS5 enhancer, extending from nucleotides −1256 to −1091, thereby deleting the enhancer MEF2 site, and a promoter extending from −944 to +7. Previous deletion studies have indicated no control elements within the −1050 to −945 MCK promoter region that was deleted. CK6 contains the full 2RS5 enhancer sequence in Example 3 FIG. 1 and a promoter extending from −358 to +7. CK4 contains the full 2RS5 enhancer and a promoter extending from −80 to +7. The CMV promoter used in plasmid constructs extends from −525 to +1 relative to its transcriptional start site. All constructs include the 150-bp minx in iron, a nuclear-targeted lacZ transgene, and the SV40 polyadenylation signal. This schematic diagram at the bottom shows an expression cassette inserted into a recombinant adenoviral vector so that transcription proceeds away from the viral left ITR. MCK regulatory elements in this orientation direct muscle-specific expression, while those in the opposite orientation allow leaky transcription in nonmuscle cells.

### DETAILED DESCRIPTION

### Abbreviations and Acronyms

AAV—adeno-associated viral/virus

Ant—anterior

CHO—Chinese hamster ovary

CM V—cytomegalovirus

cTnT—cardiac troponin-T

eGFP—enhanced green fluorescent protein

ECL—Erythrina cristagalli lectin (also used for the abbreviation of enhanced chemiluminescence

EcSOD—extracellular superoxide dismutase

EDV—end-diastole

EF—ejection fraction

eGFP—enhanced green fluorescence protein

ESV—end-systole

GA—gastrocnemius muscle

Gd-DTPA—gadolinium diethylenetriamine pentaacetic acid

GFP—green fluorescent protein

HLI—hindlimb ischemia

I—ischemic

IM—intramuscular

Inf—inferior

IR—ischemia reperfusion

ITR—inverted terminal repeat

IV—intravenous

TVIS—in vivo bioluminescence imaging

LAD—left anterior descending coronary artery

Lat—lateral

LGE—late gadolinium enhanced

LVEDV—left ventricular end-diastolic volume

LVESV—left ventricular end-systolic volume

MALI—Maackia amurensis lectin

MCK—muscle creatine kinase

MI—myocardial infarction

miRNA—microRNA

NAD—neuraminidase

NI—non-ischemic

nt—nucleotide

PAD—peripheral arterial disease

Sep—septal

TA—tibialis anterior

TNT—troponin T

vg—vector genome or viral genome

### DEFINITIONS

In describing and claiming the invention, the following terminology will be used in accordance with the definitions set forth below. Unless defined otherwise, all technical and scientific terms used herein have the commonly understood by one of ordinary skill in the art to which the invention pertains. Although any methods and materials similar or equivalent to those described herein may be useful in the practice or testing of the present invention, preferred methods and materials are described below. Specific terminology of particular importance to the description of the present invention is defined below.

The articles “a” and “an” are used herein to refer to one or to more than one (i.e., to at least one) of the grammatical object of the article. By way of example, “an element.” means one element or more than one element.

The term “about,” as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As use herein, the terms “administration of” and or “administering” a compound should be understood to mean providing a compound of the invention or a prodrug of a compound of the invention to a subject in need of treatment.

As used herein, an “agonist” is a composition of matter which, when administered to a mammal such as a human, enhances or extends a biological activity attributable to the level or presence of a target compound or molecule of interest in the subject.

As used herein, “alleviating a disease or disorder symptom,” means reducing the severity of the symptom or the frequency with which such a symptom is experienced by a subject, or both.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

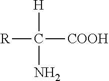

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog”, or “analogue” of a chemical compound is a compound that, by way of example, resembles another in structure but is not necessarily an isomer (e.g., 5-fluoro uracil is an analog of thymine).

An “antagonist” is a composition of matter which when administered to a mammal such as a human, inhibits a biological activity attributable to the level or presence of a compound or molecule of interest in the subject.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “associated with ischemia” as used herein means that an injury, disease, or disorder that is being treated or which is being prevented either develops as a result of ischemia or ischemia develops as a result of the injury disease or disorder, i.e., the two are closely linked.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, sputum, CSF, blood, serum, plasma, gastric aspirates, throat swabs, skin, hair, tissue, blood, plasma, serum, cells, sweat and urine,

“Blood components” refers to mam/important components such as red cells, white cells, platelets, and plasma and to other components that can be derived such as serum.

As used herein, the term “carrier molecule” refers to any molecule that is chemically conjugated to the antigen of interest that enables an immune response resulting in antibodies specific to the native antigen.

A “chamber”, as used herein, refers to something to which a solution can be added, such as a tube or well of a multiwell plate, etc.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the earner protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homo log thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above. When referring to a compound of the invention, and unless otherwise specified, the term “compound” is intended to encompass not only the specified molecular entity but also its pharmaceutically acceptable, pharmacologically active analogs, including, but not limited to, salts, polymorphs, esters, amides, prodrugs, adducts, conjugates, active metabolites, and the like, where such modifications to the molecular entity are appropriate.

As used herein, the term “conservative amino acid substitution” is defined herein as an amino acid exchange within one of the following five groups:
I. Small aliphatic, nonpolar or slightly polar residues: Ala, Ser, Thr, Pro, Gly;
II. Polar, negatively charged residues and their amides: Asp, Asn, Glu, Gin;
III. Polar, positively charged residues: Bis, Arg, Lys;
IV. Large, aliphatic, nonpolar residues: Met Leu, Ile, Val, Cys
V. Large, aromatic residues: Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

The term “delivery vehicle” refers to any kind of device or material which can be used to deliver compounds in vivo or can be added to a composition comprising compounds administered to a plant or animal. This includes, but is not limited to, implantable devices, aggregates of cells, matrix materials, gels, etc.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “defect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm which is capable of regulating a biochemical pathway.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element, that, can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most, antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

As used in the specification and the appended claims, the terms “for example,” “for instance,” “such as,” “including” and the like are meant to introduce examples that further clarify more general subject matter. Unless otherwise specified, these examples are provided only as an aid for understanding the invention, and are not meant to be limiting in any fashion.

The terms “formula” and “structure” are used interchangeably herein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” molecule is a molecule in a form in which it exhibits a property or activity by which it is characterized. A functional enzyme, for example, is one that exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a sub unit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Kariin and Altschul (1990, Proc. Natl. Acad. Sci. USA 87:2264-2268), modified as in Kariin and Altschul (1993, Proc. Natl. Acad, Sci, USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “biastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHl-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “inhibit,” as used herein, refers to the ability of a vector, transgene, or compound of the invention to reduce or impede a described function. Preferably, inhibition is by at least 10%, more preferably by at least 25%, even more preferably by at least 50%, and most preferably, the function is inhibited by at least 75%. The terms “inhibit”, “reduce”, and “block” are used interchangeably herein.

The term “inhibit a complex,” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the invention by any number of routes and means including, but not limited to, systemic, enteral, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “ischemia” as used herein refers to a local anemia due to mechanical obstruction of the blood supply, which gives rise to inadequate circulation of the blood to an organ, tissue, or region of an organ or tissue.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

“Left ventricle remodeling associated with an injury, disease, or disorder” means change or repair in the left ventricle of the heart. In lower animals with different chambers the remodeling may be in a different chamber.

A “ligand” is a compound that specifically binds to a target receptor.

A “receptor” is a compound that specifically binds to a ligand.

A ligand or a receptor (e.g., an antibody) “specifically binds to” or “is specifically immune-reactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process.

The term “muscle-specific” is used, where appropriate, interchangeably with “tissue-specific” or “tissue-preferential” and refers to the capability of regulatory elements, such as promoters and enhancers, to drive expression of transgenes exclusively or preferentially in muscle tissue or muscle cells regardless of their source.

The term “myocyte,” as used herein, refers a cell that has been differentiated from a progenitor myoblast such that it is capable of expressing muscle-specific phenotype under appropriate conditions. Terminally differentiated myocytes fuse with one another to form myotubes, a major constituent of muscle fibers. The term “myocyte” also refers to myocytes that are de-differentiated. The term includes cells in vivo and cells cultured ex vivo regardless of whether such cells are primary or passaged.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include nitrons.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “per application” as used herein refers to administration of a compositions, drug, or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically acceptable carrier” includes any of the standard pharmaceutical earners, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder,

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability or be easier to formulate.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with, a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit, signs of a disease or exhibits only early signs of the disease for the purpose of decreasing the risk of developing pathology associated with the disease, or is done before a specific surgical procedure, etc.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxy 1-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “receptor” is a compound that specifically binds to a ligand.

A “ligand” is a compound that specifically binds to a target receptor.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

A “recombinant adeno-associated viral (AAV) vector comprising a regulatory element active in muscle cells” refers to an AAV that has been constructed to comprise a new regulatory element to drive expression or tissue-specific expression in muscle of a gene of choice or interest. As described herein such a constructed vector may also contain at least one promoter and optionally at least one enhancer as part of the regulatory element, and the recombinant vector may further comprise additional nucleic acid sequences, including those for other genes, including therapeutic genes of interest.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “reporter gene” means a gene, the expression of which can be detected using a known method. By way of example, the Escherichia coli lacZ gene may be used as a reporter gene in a medium because expression of the lacZ gene can be defected using known methods by adding the chromogenic substrate o-nitrophenyl-β-galactoside to the medium (Gerhardt et al, eds., 1994, Methods for General and Molecular Bacteriology, American Society for Microbiology, Washington, D.C., p. 574).

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this invention.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%), 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide that has been separated from components which naturally accompany it. Typically, a compound is substantially pure when at least 10%, more preferably at least 20%, more preferably at least 50%, more preferably at least 60%, more preferably at least 75%), more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term “transfection” is used interchangeably with the terms “gene transfer”, transformation,” and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

The term “transgene” is used interchangeably with “inserted gene,” or “expressed gene” and, where appropriate, “gene”. “Transgene” refers to a polynucleotide that, when introduced into a cell, is capable of being transcribed under appropriate conditions so as to confer a beneficial property to the cell such as, for example, expression of a therapeutically useful protein. It is an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

Where appropriate, the term “transgene” should be understood to include a combination of a coding sequence and optional non-coding regulatory sequences, such as a polyadenylation signal, a promoter, an enhancer, a repressor, etc.

The term to “treat,” as used herein, means reducing the frequency with which symptoms are experienced by a patient or subject or administering an agent or compound to reduce the frequency with which symptoms are experienced. As used herein, the term “treating” can include prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat,” as used herein, means reducing the frequency with which symptoms are experienced by a patient or subject or administering an agent or compound to reduce the frequency with which symptoms are experienced.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphophilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant, polynucleotide comprising expression control sequences operatively linked to a nucleotide sequence to be expressed. An expression vector comprises sufficient cis-acting elements for expression; other elements for expression can be supplied by the host, cell or in an in vitro expression system. Expression vectors include all those known in the art, such as cosmids, plasmids (e.g., naked or contained in liposomes) and viruses that incorporate the recombinant polynucleotide.

### Embodiments

The present invention relates to compositions and methods for targeting muscle with adeno-associated viral vectors comprising useful regulatory elements for achieving expression of genes of interest. In one aspect, the vector further comprises a gene of interest, which may be a therapeutic gene. The regulatory element may include an additional enhancer and/or a promoter. In one aspect, the enhancer and/or promoter are tissue specific for muscle, and may be specific for cardiac myocytes or for skeletal myocytes. The method is useful for treating various injuries, diseases, and disorders of muscle. The combination of specific AAV vectors, enhancers, promoters, and therapeutic genes of interest that are used can be modified to ensure a higher rate of targeting of cells and tissues of interest and expression of therapeutic genes and genes of interest in the target cell of tissue of interest.

In one embodiment the muscle is cardiac muscle. In another embodiment, the muscle is skeletal muscle.

In one aspect, the subject animal is a mammal. In one aspect, the mammal is a human. The compositions and methods of the invention can be used on many types of animals, including livestock, pets, birds, cats, dogs, reptiles, and amphibians, including animals in zoos.

Other useful vectors, nucleic acids, and proteins or homologs and fragments thereof are useful with the practice of the invention, including but not limited to:

AAV-9—NCBI Accession number AX753250;

AAV-8—NCBI Accession number NC 006261;

Mouse therapeutic cDNA 1: Sod3 (EC-SOD) NCBI Accession number NM_{—}011435;

Human therapeutic cDNA 1: SOD3 (EC-SOD) NCBI Accession number NP_{—}003102;

Mouse therapeutic gene 1: Sod3 (EC-SOD) Gene ID 20657;

Human therapeutic gene 1: SOD3 (EC-SOD) Gene ID 6649;

Chicken promoter 1—TNNT2 (cardiac troponin T type 2) Gene ID 396433; and

Human promoter 1—TNNT2 (cardiac troponin T type 2) Gene ID 7139.

The human muscle creatine kinase gene has Gene ID: 1158 (GenBank). The protein for SEQ ID NO:11 (AAV8) is capsid protein gpl and has GenBank accession number YP_{—}077179.1.

In some experiments mouse cDNA can be used to avoid generating a foreign antigen in mice for testing new vectors, but in some cases of treatment the human cDNA is preferred. Due to the payload constraints of AAV, in one embodiment a cDNA may be preferred. In one aspect, additional introns and sequences can be introduced. In one aspect, the cap gene of the AAV is used and not the entire AAV genomic DNA.

Other methods and vectors are known in the art which could also be used to practice the methods of the present invention, including those in Souza et al. (U.S. Pat, Pub. No. 2011/0212529, published Sep. 1, 2011).

Although AAVs such as AAV9 and AAV8 may target some tissues with higher specificity than other tissues, the use of tissue or eel 1 specific enhancers and promoters as part of the vector can help to ensure that the genes of interest are expressed in the desired cell or tissue,

Ordahl et al. (U.S. Pat. No. 5,266,488) characterized the chicken troponin-T gene promoter and found the essential proximal promoter element contains nonspecific sequences necessary for the initiation of transcription of a structural gene to be operatively associated with the promoter. See FIG. 2 of Ordahl and SEQ ID NO:18 herein. When +1 designates the first nucleotide of the transcription initiation site, this element is located between nucleotide −49 and nucleotide +1. Further, Ordahl demonstrated that the skeletal muscle-specific regulatory element is positioned upstream of the essential proximal promoter element and is operationally associated therewith. This element is necessary for the expression of a structural gene to be operatively associated with the promoter in skeletal muscle cells. The skeletal muscle-specific regulatory element is located between nucleotide −129 and −49. Ordahl also stated that the cardiac muscle-specific regulatory element is positioned upstream of both the skeletal muscle specific regulatory element and the essential proximal promoter element and is operatively associated with the essential proximal promoter element and suggested that this element is necessary for the expression of a structural gene to be operatively associated with the promoter in cardiac muscle cells. Ordahl also asserted that the cardiac muscle-specific regulatory element is located between nucleotide −268 and nucleotide −201.

Ordahl also demonstrated that the nonessential positive striated muscle regulatory element is positioned upstream of, and operationally associated with, both the skeletal muscle specific regulatory element and the cardiac muscle-specific regulatory element. This element facilitates the expression of a structural gene to be operatively associated with the promoter in striated muscle cells, both cardiac and skeletal. This element is located between nucleotide −550 and −269.

According to Ordahl, the nonessential negative regulatory element is positioned upstream of the positive striated muscle regulatory element and is operatively associated therewith. This element inhibits the positive striated muscle regulatory element from facilitating the expression of a structural gene to be operatively associated with the promoter. This element is located between nucleotide −3000 and nucleotide −1100. More broadly defined, this element is located between nucleotide −3000 and nucleotide −550.

In one embodiment, the present invention encompasses the use of the promoter regions described by Ordahl for targeting muscle in general or for more specifically targeting cardiac muscle over skeletal muscle or vice-versa.

A complete promoter (one containing all the elements described above) expresses a structural gene operatively associated therewith in both skeletal and striated muscle cells. The individual elements which comprise a complete promoter can be used in any desired operable combination to produce new promoters having different properties. For example, the negative nonspecific regulatory element can be deleted from a complete promoter so that the expression of a gene associated with the promoter is facilitated. The cardiac muscle-specific regulatory element can be deleted from a complete promoter so that a structural gene operatively associated with the promoter is preferentially expressed in skeletal cells, or the skeletal muscle-specific regulatory element can be deleted from a complete promoter so that a structural gene operatively associated with the promoter is preferentially expressed in cardiac cells. The term “deleted,” as used herein, means any modification to a promoter element which renders that element inoperable.

Operable promoters can be constructed from the minimum necessary regulatory elements. One such promoter comprises an essential proximal promoter element and a cardiac muscle-specific regulatory element positioned upstream of the essential proximal promoter element and operatively associated therewith. Another such promoter comprises an essential proximal promoter element and a skeletal muscle-specific regulatory element positioned upstream of said essential proximal promoter element and operatively associated therewith. To these promoters, a positive striated muscle regulatory element may optionally be positioned upstream oft and operatively associated with, the specific regulatory element (skeletal or cardiac).

Therefore, the present invention encompasses the use of a cardiac troponin-T promoter, for example, where the sequence comprises a promoter and is the 5′ region of about nucleotide position −3000 to about the transcription start site of cardiac troponin-T or about nucleotide +25 to about +50, or where the sequence comprises the 5′ region of about nucleotide −1000 to about the transcription start site or about nucleotide +25 to about +50, or where the sequence comprises the 5′ region of about nucleotide −550 to about the transcription start site or about nucleotide +25 to about +50, or where the sequence comprises the 5′ region of about nucleotide −400 to about the transcription start site or about nucleotide +25 to about +50, or where the sequence comprises the 5′ region of about nucleotide −300 to about the transcription start site or about nucleotide +25 to about +50. In one aspect, the sequence is about 375 nucleotides upstream (−) to 43 nucleotides downstream (+) (see Example 1). In another aspect, the sequence is 5′ region from about nucleotide −268 to about nucleotide +38 relative to the transcription start site (SEQ ID NO:18).

It will be understood by one of ordinary skill in the art that when a different promoter is being used, such as a muscle creatine kinase promoter, similar to the cardiac troponin-T promoter various lengths of the sequence can also be used.

In one embodiment, the present invention encompasses compositions and methods for transducing skeletal muscle and enhancing gene expression using an AAV vector engineered to comprise a skeletal muscle gene promoter. In one aspect, the AAV is AAV9 or AAV8. In one aspect, AAV9 comprises the nucleic acid sequence of SEQ ID NO:1. In one aspect, AAV8 comprises the nucleic acid sequence of SEQ ID NO:11. The compositions and methods of the invention encompass the use of all or parts of SEQ ID NOs:1 and 11. In one aspect, the promoter is a muscle creatine kinase promoter. In one aspect, the muscle creatine kinase promoter is a human promoter. In another aspect, it is a murine promoter. In one aspect, the promoter is found in murine SEQ ID NO:4. In one aspect, the invention encompasses the use of the 319 bp sequence of chicken cardiac troponin-T promoter of GenBank Accession No. M579G5.1, which comprises exon 1 and a promoter sequence. The present invention further encompasses the use of fragments of the sequences described herein wherein the fragments maintain the described function.

In one embodiment, the present invention relates to gene therapy methods utilizing tissue-specific expression vectors. The invention further relates to expression vectors used for delivery of a transgene into muscle. In one aspect, the muscle is cardiac muscle. In another aspect, the muscle is skeletal muscle. More specifically, the invention relates to transcriptional regulatory elements that provide for enhanced and sustained expression of a transgene in the muscle.

Skeletal muscle promoters and enhancers are available for the muscle creatine kinase (MCK) gene and are encompassed by the presented invention for regulating expression of a therapeutic gene in an AAV vector of the invention. For example, in one aspect, an enhancer of the invention comprises SEQ ID NO:15, which can also be used in combination with a promoter sequence of MCK such as the −358 to +7 sequence. When the 206 bp SEQ ID NO:15 sequence and the 365 bp promoter stretch of −358 to +7 are combined the 571 bp CK6 promoter/enhancer of the invention is obtained. The present invention further encompasses the use of 5′ region from about −1000 to about +7, from about −500 to about +7, from about −400 to about +7, from about −300 to about +7, from about −200 to about +7, from about −100 to about +7, and from about −80 to about +7.

Other skeletal muscle promoters and enhancers can also be incorporated into an AAV vector of the invention.

Accordingly, one embodiment of the invention provides expression vectors optimized for sustained expression of a transgene in muscle tissue. Another object of this invention is to provide enhancer/promoter combinations that can direct sustained and appropriate expression levels in various expression systems.

In one embodiment, the invention encompasses combining minimal sequences from muscle-specific promoters and muscle-specific enhancers to create chimeric regulatory elements that drive transcription of a transgene in a sustained fashion. A minimal sequence is one which maintains the function of interest, although possibly somewhat less than the full sequence of interest. The resulting chimeric regulatory elements are useful for gene therapy directed at transgene expression in the muscle as well as other applications requiring long-term expression of exogenous proteins in transfected muscle cells such as myocytes. In one aspect, the myocytes are cardiac myocytes. In another aspect, the myocytes are skeletal muscle myocytes.

Chimeric regulatory elements useful for targeting transgene expression to the muscle are provided by the invention. The chimeric regulatory elements of the invention comprise combinations of muscle-specific promoters and muscle-specific enhancers that are able to direct sustained transgene expression preferentially in the muscle. In one aspect, the enhancers and promoters are cardiac specific and in another aspect, the enhancers and promoters are skeletal muscle specific.

The present invention is also directed to recombinant transgenes which comprise one or more operably linked tissue-specific regulatory elements of the invention. The tissue-specific regulatory elements, including muscle-specific promoter and enhancers operably linked to a transgene, drive its expression in myocytes and, in particular, in cardiomyocytes and/or skeletal myocytes. The transgenes may be inserted in recombinant viral vectors for targeting expression of the associated coding DNA sequences in muscle. Muscle-specific promoters useful in the invention include, for example, muscle creatine kinase (MCK) promoter, cardiac troponin-T promoter, or desmin (DES) promoter. In one particular embodiment, the promoter is a human promoter. In another embodiment, the promoter is a murine promoter. In yet another embodiment, the promoter is a chicken promoter. In certain embodiments, the promoter is truncated.

In one embodiment, tissue-specific enhancers are used. Tissue-specific enhancers include muscle specific enhancers. One or more of these muscle-specific enhancer elements may be used in combination with a muscle-specific promoter of the invention to provide a tissue-specific regulatory element. In one embodiment, the enhancers are derived from human, chicken, or mouse. In certain embodiments, the enhancer/enhancer or enhancer/promoter combinations are heterologous, i.e., derived from more than one species. In other embodiments, the enhancers and promoters are derived from the same species. In certain embodiments, enhancer elements are truncated.

In one embodiment, a regulatory element of the invention comprises at least one MCK or cardiac troponin-T enhancer operably linked to a promoter. In another embodiment, a regulatory element of the invention comprises at least two MCK enhancers linked to a MCK promoter or a DES promoter or a cardiac troponin-T promoter. In yet another embodiment, a regulatory element comprises at least two DES enhancers linked to a promoter. In a further embodiment, a regulatory element comprises at least two cardiac troponin-T enhancers linked to a promoter.

The invention provides vectors comprising a regulatory element of the invention. In some embodiments, a regulatory element of the invention is incorporated into a viral vector such as one derived from adenoviruses, adeno-associated viruses (AAV), or retroviruses, including lentiviruses such as the human immunodeficiency (HIV) virus. In one embodiment, the AAV is AAV8 or AAV9. The invention also encompasses methods of transfecting muscle tissue where such methods utilize the vectors of the invention.

The invention further provides cells transfected with the nucleic acid containing an enhancer/promoter combination of the invention.

Promoters may be coupled with other regulatory sequences/elements which, when bound to appropriate intracellular regulatory factors, enhance (“enhancers”) or repress (“repressors”) promoter-dependent transcription. A promoter, enhancer, or repressor, is said to be “operably linked” to a transgene when such element(s) control(s) or affect(s) transgene transcription rate or efficiency. For example, a promoter sequence located proximally to the 5′ end of a transgene coding sequence is usually operably linked with the transgene. As used herein, term “regulatory elements” is used interchangeably with “regulatory sequences” and refers to promoters, enhancers, and other expression control elements, or any combination of such elements.

Promoters are positioned 5′ (upstream) to the genes that they control. Many eukaryotic promoters contain two types of recognition sequences: TATA box and the upstream promoter elements. The TATA box, located 25-30 bp upstream of the transcription initiation site, is thought to be involved in directing RNA polymerase II to begin RNA synthesis as the correct site. In contrast, the upstream promoter elements determine the rate at which transcription is initiated. These elements can act regardless of their orientation, but they must be located within 100 to 200 bp upstream of the TATA box.

Enhancer elements can stimulate transcription up to 1000-fold from linked homologous or heterologous promoters. Enhancer elements often remain active even if their orientation is reversed (Li et al., J. Bio. Chem. 1990, 266: 6562-6570). Furthermore, unlike promoter elements, enhancers can be active when placed downstream from the transcription initiation site, e.g., within an intron, or even at a considerable distance from the promoter (Yutzey et al., Mol. and Cell. Bio. 1989, 9:1397-1405).

It is known in the art that some variation in this distance can be accommodated without loss of promoter function. Similarly, the positioning of regulatory elements with respect to the transgene may vary significantly without loss of function. Multiple copies of regulatory elements can act in conceit. Typically, an expression vector comprises one or more enhancer sequences followed by, in the 5′ to 3′ direction, a promoter sequence, all operably linked to a transgene followed by a polyadenylation sequence.

The present invention further relies on the fact that many enhancers of cellular genes work exclusively in a particular tissue or cell type. In addition, some enhancers become active only under specific conditions that are generated by the presence of an inducer such as a hormone or metal ion. Because of these differences in the specificities of cellular enhancers, the choice of promoter and enhancer elements to be incorporated into a eukaryotic expression vector is determined by the cell type(s) in which the recombinant gene is to be expressed.

In one aspect, the regulatory elements of the invention may be heterologous with regard to each other or to a transgene, that is, they may be from different species. Furthermore, they may be from species other than the host, or they also may be derived from the same species but from different genes, or they may be derived from a single gene.

The present invention further includes the use of desmin regulatory elements. Desmin is a muscle-specific cytoskeletal protein that belongs to the family of intermediate filaments that occur at the periphery of the Z disk and may act to keep adjacent myofibrils in lateral alignment. The expression of various intermediate filaments is regulated developmentally and shows tissue specificity.

The muscle creatine kinase (MCK) gene is highly active in all striated muscles. Creatine kinase plays an important role in the regeneration of ATP within contractile and ion transport systems. It allows for muscle contraction when neither glycolysis nor respiration is present by transferring a phosphate group from phosphocreatine to ADP to form ATP. There are four known isoforms of creatine kinase: brain creatine kinase (CKB), muscle creatine kinase (MCK), and two mitochondrial forms (CKMi). MCK is the most abundant non-mitochondrial mRNA that is expressed in all skeletal muscle fiber types and is also highly active in cardiac muscle. The MCK gene is not expressed in myoblasts, but becomes transcriptionally activate when myoblasts commit to terminal differentiation into myocytes. MCK gene regulatory regions display striated muscle-specific activity and have been extensively characterized in vivo and in vitro. Mammalian MCK regulatory elements are described, for example, in Hauser et al., Mol. Therapy. 2000, 2; 16-25 and in Souza et al., 2011. MCK enhancer and promoter sequences are provided herein.

The present invention further includes the use of troponin regulatory elements, particularly cardiac troponin.

The present invention further includes the use of combinations of elements to form, for example, chimeric regulatory elements. The present invention is directed to recombinant transgenes which comprise one or more of the tissue-specific regulatory elements described herein. The chimeric tissue-specific regulatory elements of the invention drive transgene expression in muscle cells. In one aspect the muscle cell is a skeletal muscle cell. In one aspect, the muscle cell is a cardiomyocyte. The transgenes may be inserted in recombinant viral or non-viral vectors for targeting expression of the associated coding DNA sequences in muscle. In one aspect, the viral vector is an AAV. In one embodiment, the promoter element is selected from the group consisting of muscle creatine kinase (MCK) promoter, desmin promoter, and cardiac troponin T promoter. In one particular embodiment, the promoter is a human promoter. In another embodiment, the promoter is a murine promoter. In another embodiment, the promoter is a chicken promoter. In certain embodiments, the promoter is truncated. One of ordinary skill in the art will appreciate that the entire promoter need not necessarily be used in all cases and that activity can be maintained when some nucleotides are deleted or added.

In one embodiment, a regulatory element of the invention comprises at least one MCK enhancer operably linked with a DES promoter or an MCK promoter or a cardiac troponin-T promoter. In another embodiment, the regulatory element comprises at least two MCK enhancers linked to a MCK promoter or a DES promoter or a cardiac troponin-T promoter. In yet another embodiment, a regulatory element comprises at least two DES enhancers linked to a DES promoter. In yet another embodiment, a regulatory element comprises at least two cardiac troponin-T enhancers linked to a cardiac troponin-T promoter. In one aspect, the MCK enhancer comprises the sequence of SEQ ID NO:15 or an active fragment or modification thereof.

It will be understood that the regulatory elements of the invention are not limited to specific sequences referred to in the specification but also encompass their structural and functional analogs/homologues and functional fragments thereof. Such analogs may contain truncations, deletions, insertions, as well as substitutions of one or more nucleotides introduced either by directed or by random mutagenesis. Truncations may be introduced to delete one or more binding sites for known transcriptional repressors. Additionally, such sequences may be derived from sequences naturally found in nature that exhibit a high degree of identity to the sequences in the invention. In one aspect, a nucleic acid of 20 nt or more will be considered to have high degree of identity to a promoter/enhancer sequence of the invention if it hybridizes to such promoter/enhancer sequence under stringent conditions. Alternatively, a nucleic acid will be considered to have a high degree of identity to a promoter/enhancer sequence of the invention if it comprises a contiguous sequence of at least 20 nt, which has percent identity of at least 60%, 65%, 70%, 75%, 80%, 85%, 90%, 95%, or more as determined by standard alignment algorithms such as, for example, Basic Local Alignment Tool (BLAST) described in Altschul et al., J. Mol. Biol. 1990, 215: 403-410, the algorithm of Needleman et al., J. Mol. Biol. 1970, 48: 444-453, or the algorithm of Meyers et al., Comput. Appl. Biosci. 1988, 4: 11-17. Non-limiting examples of analogs, e.g., homologous promoter sequences and homologous enhancer sequences derived from various species, are described in the present application.

In one embodiment, the invention further includes vectors comprising a regulatory element of the invention. In general, there are no known limitations on the use of the regulatory elements of the invention in any vector. A regulatory element comprises a promoter element and optionally an enhancer element.

In the present invention, the therapeutic transgene may comprise a DNA sequence encoding proteins involved in metabolic diseases, or disorders and diseases of muscle system, muscle wasting, or muscle repair. Vectors of the invention may include a transgene containing a sequence coding for a therapeutic polypeptide. For gene therapy, such a transgene is selected based upon a desired therapeutic outcome. It may encode, for example, antibodies, hormones, enzymes, receptors, or other proteins of interest or their fragments, such as, for example, TGF-beta receptor, glucagon-iike peptide 1, dystrophin, leptin, insulin, pre-proinsulin, follistatin, PTH, FSH, IGF, EGF, TGF-beta, bone morphogenetic proteins, other tissue growth and regulatory factors, growth hormones, and blood coagulation factors.

The invention encompasses methods of transfecting the muscle tissue where such methods utilize the vectors of the invention. It will be understood that vectors of the invention are not limited by the type of the transfection agent in which to be administered to a subject or by the method of administration. Transfection agents may contain compounds that reduce the electrostatic charge of the cell surface and the polynucleotide itself, or increase the permeability of the cell wall. Examples include cationic liposomes, calcium phosphate, polylysine, vascular endothelial growth factor (VEGF), etc. Hypertonic solutions containing, for example, NaCl, sugars, or polyols, can also be used to increase the extracellular osmotic pressure thereby increasing transfection efficiency. Transfection agents may also include enzymes such as proteases and lipases, mild detergents and other compounds that increase permeability of cell membranes. The methods of the invention are not limited to any particular composition of the transfection agent and can be practiced with any suitable agent so long as it is not toxic to the subject or its toxicity is within acceptable limits.

The invention also includes cells transfected with the DNA containing an enhancer/promoter combination of the invention. Standard methods for transfecting cells with isolated nucleic acids are well known to those skilled in art. Transfected cells may be used, for example, to confirm the identity of a transgene; to study biosynthesis and intracellular transport of proteins encoded by transgenes; or to culture cells ex vivo for subsequent re-implantation into a subject, etc. Methods for in vivo intramuscular injection and transfection of myocytes ex vivo are known in the art. For example, see Shah et al., Transplantation 1999, 31: 641-642; Daly et al, Human Gene Therapy 1999, 10:85-94.

Host cells that can be used with the vectors of invention include myocytes. Myocytes include those found in all muscle types, e.g., skeletal muscle, cardiac muscle, smooth muscle, etc. Myocytes are found and can be isolated from any vertebrate species, including, without limitation, human, orangutan, monkey, chimpanzee, dog, cat, rat, rabbit, mouse, horse, cow, pig, elephant, etc. Alternatively, the host cell can be a prokaryotic cell, e.g., a bacterial cell such as E. coli that is used, for example, to propagate the vectors.

In one embodiment, the present invention provides for the use of myocyte progenitor cells such as mesenchymal precursor cells or myoblasts rather than fully differentiated myoblasts. Examples of tissue from which such cells can be isolated include placenta, umbilical cord, bone marrow, skin, muscle, periosteum, or perichondrium. Myocytes can be derived from such cells, for example, by inducing their differentiation in tissue culture or upon transplantation. The present invention encompasses not only myocyte precursor/progenitor cells, but also cells that can be trans-differentiated into myocytes, e.g., adipocytes and fibroblasts.

In one embodiment, the AAV vectors of the invention may further contain a nucleic acid sequence encoding a therapeutic gene or protein.

In one embodiment, the AAV vector can be injected into an embryo so that the expression of transgene is suppressed until some stage in development when myocytes have been differentiated. See, e.g., Gene Expression Systems, Eds. J. M. Fernandez and J. P. Hoeffler, Academic Press, San Diego, Calif., 1999.

The invention further provides methods for determining magnitude of expression and AAV genome copy number. Such methods are useful for verification of the targeted cell or tissue of interest being transduced and how much of the AAV vector is present, as well as how much the gene of interest or therapeutic gene is being expressed.

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; miRNA, siRNA, and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

miRNAs are RNA molecules of about 22 nucleotides or less in length. These molecules are post-transcriptional regulators that bind to complementary sequences on target mRNAs. Although miRNA molecules are generally found to be stable when associated with blood serum and its components after EDTA treatment, introduction of locked nucleic acids (LNAs) to the miRNAs via PGR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules,

A composition of the invention may comprise additional ingredients. As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The pharmaceutical composition may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the condition or disease being treated, the type and age of the animal, etc.

In other embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes or other agents may be used as adjunct therapies.

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled. DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

Other embodiments of the invention will be apparent to those skilled in the art based on the disclosure and embodiments of the invention described herein. It is intended that the specification and examples be considered as exemplary only, with a true scope and spirit of the invention being indicated by the following claims. While some representative experiments have been performed in test animals, similar results are expected in humans. The exact parameters to be used for injections in humans can be easily determined by a person skilled in the art.

The invention is now described with reference to the following Examples and Embodiments. Without further description, it is believed that one of ordinary skill in the art can, using the preceding description and the following illustrative examples, make and utilize the present invention and practice the claimed methods. The following working examples therefore, are provided for the purpose of illustration only and specifically point out the preferred embodiments of the present invention, and are not to be construed as limiting in any way the remainder of the disclosure. Therefore, the examples should be construed to encompass any and all variations which become evident as a result of the teaching provided herein.

### Example 1

### AAV9 Administered Systemically After Reperfusion Preferentially Targets Cardiomyocytes in the Infarct Border Zone with Pharmacodymanics Suitable for the Attenuation of Left Ventricular Remodeling

Materials and Methods

Plasmids: The AAV vectors containing the 418 bp chicken cardiac troponin-T (cTnT) promoter driving the expression of firefly luciferase (AcTnTLuc), eGFP (AcTnTeGFP) or EcSOD (AcTnTEcSOD) are diagrammed in Example 1, FIG. 1, and their construction has previously been described. The 418 bp chicken cTnT promoter spans from 375 nucleotides upstream (−) to 43 nucleotides downstream (+) of the cTnT transcriptional start site. The chicken cardiac troponin-T cDNA has SEQ ID NO:2 (GenBank Accession No. NM_{—}205449.1) and the gene has Gene ID: 396433. The gene is located on chromosome 26 and the 8.28 kb region from base 789127 to 797492 has GenBank Accession No. NC 006113.3 (8276 bp). SEQ ID NO:18 is a 306 bp chicken cardiac troponin-T 5′ region from −268 to +38 relative to the transcription start site (see also FIG. 2 of U.S. Pat. No. 5,266,488).

Cardiac troponin-T promoters from other species have been identified and the various regions of the promoters have been studied (Harlan et al., 2008, Anat. Rec, 291:12:1574; March et al., 1988, Proc. Natl. Acad. Sci., 85:6404; Ordahl et al., U.S. Pat. No. 5,266,488; Prasad et al., J. Gene Medicine, 2011, 13:333; Prasad et al, Gene Ther., 2011, 18:1:43; Tidyman et al., Developmental Dynamics, 2003, 227:484; March et al., 1988, J. Cell Biol, 107:573; Iannello, 1991, J. Biol. Chem., 266:5:3309; Cooper and Ordahl, J. Biol. Chem., 1985, 260:20:11140).

AAV vector production: AAV2-based vector genomes were cross-packaged into AAV9 capsids via the triple transfection of HEK 293 cells, then purified by ammonium sulfate fractionation and iodixanol gradient centrifugation. Titers of the AAV vectors (viral genomes/ml) were determined by quantitative real-time PCR. The following primers were used for amplifying luciferase—

SEQ ID NO: 5
5′-AGAACTGCCTGCGTGAGATT-3′ (forward)
and
SEQ ID NO: 6
5′-AAAACCGTGATGGAATGGAA-3′ (reverse);
SEQ ID NO: 7
eGFP: 5′-CACATGAAGCAGCACGACTT-3 (forward)
and
SEQ ID NO: 8
5′-GAAGTTCACCTTGATGCCGT-3′ (reverse);
and
SEQ ID NO: 9
EcSOD: 5′-CCTAGCAGACAGGCTTGACC-3′ (forward)
and
SEQ ID NO: 10
5′-CCATCCAGATCTCCAGCACT-3′ (reverse).

Known copy numbers (105-108) of the respective plasmids carrying the corresponding cDNAs were used to construct standard curves for quantification.

Myocardial IR and vector administration: Animal protocols used in the study were approved by the Institutional Animal Care and Use Committee and conformed to the “Guide for the Care and Use of Laboratory Animals” (NIB Publication 85-23, revised 1985). C57BL/6 mice (8-10 weeks old, weighing 20-25 g) were purchased from The Jackson Laboratories (Bar Harbor, Me.) and maintained on a 12/12 hr light/dark cycle at 24° C. and 60% humidity. The procedure employed to induce myocardial IR injury in mice has been described previously. Briefly, mice were anesthetized with intraperitoneal (IP) injected sodium pentobarbital (TOO mg/kg) and orally intubated. Artificial respiration was maintained at 80% inspired oxygen by using 100 strokes/mm and a 2-3 ml tidal volume delivered through a loose connection from the rodent ventilator. The hearts were exposed through a left thoracotomy. Left anterior descending coronary artery (LAD) occlusion was accomplished by passing a suture beneath the LAD and tightening it over a piece of polyethylene-60 tubing. The LAD was occluded for 30 minutes in the preliminary studies of reporter gene expression and for 60 minutes in the LV remodeling study. Reperfusion was induced by removing the piece of tubing. For IV injection, mice were anesthetized with 1-1.2% isoflurane in oxygen while viral solution (50 μl containing 1×10¹¹ viral genome particles in all studies) was slowly injected via the jugular vein.

Bioluminescence imaging: Luciferase expression was serially assessed in live mice using an in vivo bioluminescence imaging system (IVIS100 system, Caliper Life Sciences, Hopkinton, Mass.) as described previously.

Quantitative luciferase activity assay: In the serial study, whole hearts were collected from mice after bioluminescence imaging and euthanasia at 7 weeks post-vector injection for luciferase activity assays. To compare the magnitude of gene expression between the previously ischemic and remote regions in mice injected 10 min post-reperfusion, the ischemic and remote zones of hearts explanted five days after vector injection were separated under a dissecting microscope for luciferase activity assays. Remote samples were obtained from the region furthest removed from the infarct (i.e., the basal septum). Luciferase activities (relative light units, RLU) in protein extracts from these tissues were determined using luciferase assay reagents from Promega Corp. (Madison, Wis.) and a FLUOstar Optima micro-plate reader (BMG Labtech, Durham, N.C.).

Determination of AAV vector genome copy number: Total genomic DNA was prepared from the mouse hearts by standard phenol-chloroform extraction. AAV vector genome copy numbers were determined by real-time quantitative PGR using the QuantiTect SYBR Green PGR kit (Qiagen Inc., Valencia, Calif.) and a Bio-Rad iCycler system (Bio-Rad Laboratories, Hercules, Calif.). The following primers were used for amplifying luciferase: SEQ ID NO:5-5′-AGAACTGCCTGCGTGAGATT-3′ (forward) and SEQ ID NO:6-5′-AAAACCGTGATGGAATGGAA-3′ (reverse). These are the same primers described above for amplifying luciferase. Known copy numbers (103-108) of the plasmid p AcTnTLuc were used to construct the standard curve. Results are expressed as the number of vector genomes per μg of genomic DNA.

Histology and immunohistochemistry: Immunostaining for eGFP protein was performed on 6 μm fixed-frozen sections. Five days following vector administration, animals were euthanized and hearts were collected and fixed in 3.7% para-formaldehyde for 1 h at 4° C. After washing in PBS (3 times, 5 min each), hearts were equilibrated with 30% sucrose in PBS overnight prior to freezing and sectioning. After incubation with hydrogen peroxide (0.5%) followed by avidin blocking, the sections were incubated overnight at 4° C. with rabbit anti-GFP antibody (1:3000 dilution, Abeam Inc., Cambridge, Mass.). Biotinylated secondary antibody (5 μg/ml, Vector Laboratories, Burlingame, Calif.) was then applied for 1 h at room temperature. After washing and incubation with avidin-biotin complex (Vector Laboratories), immunoreactivity was visualized by incubating the sections with the chromogen 3,3-diaminobenzidine tetrahydrochloride (DAB, Dako, Carpinteria Calif.) to produce a brown precipitate. Immunostained sections were counterstained with eosin before they were coverslipped for photography. Hearts were processed similarly to immunostain cardiomyocytes with a rabbit polyclonal antibody against myoglobin (Dako) using a Cy5-labeled goat anti-rabbit IgG (Life Technologies, Grand Island, N.Y.) as secondary antibody. In the LV remodeling study, conventional hematoxylin and eosin (H&E) straining was performed on heart sections obtained 4 weeks post-MI.

Western immunoblotting: Flash-frozen tissue samples were homogenized in RIPA buffer, and equal amounts of protein (as determined by Bio-Rad De Protein Assay) were electrophoresed under reducing conditions on a polyacryl amide gel and then transferred onto PVDF membranes. After blocking, membranes were incubated overnight at 4° C. with goat anti-GFP (BA-0702, Vector Laboratories Inc., Burlingame, Calif.) or rabbit anti-EcSOD (07-704, EMD Millipore Corp., Billerica, Mass.) followed by 1-h incubation at room temperature with rabbit anti-goat IgG conjugated with horseradish peroxidase (sc-2768, Santa Cruz Biotechnology Inc., Santa Cruz, Calif.) or goat anti-rabbit IgG conjugated with fluorescent dye (926-32211, LI-COR Biosciences, Lincoln, Nebr.). Membranes were imaged via chemiluminescence or fluorescence. To control for protein loading, GFP membranes were stripped and reprobed overnight at 4° C. with rabbit anti-actin antibody (A2.103, Sigma-Aldrich Inc, St. Louis, Mo.), followed by I-h incubation at room temperature with goat anti-rabbit IgG conjugated with horseradish peroxidase (170-6615, Bio-Rad Laboratories). Similarly, EcSOD membranes were stripped and reprobed overnight at 4CC with rabbit anti-GAPDH antibody (600-401-A33, Rockland Immunochemicals Inc., Gilbertsville, Pa.), followed by 1-h incubation at room temperature with fluorescently-labeled goat anti-rabbit IgG. Signal intensities on Western blots were quantified by densitometry using ImageJ (NIH, Bethesda Md.) and the primary signal in each lane was normalized to the loading control before being graphed relative to the mean of the negative control lanes. Evaluation of cardiac function by echocardiography:

A total of 17 mice were subjected to 60 min of coronary occlusion. Ten minutes following reperfusion, 8 mice were injected IV with AcTnTEcSOD while the remaining 9 mice served as controls. The procedure employed here to induce myocardial IR injury was the same as that described in “Myocardial IR and vector administration” except with reperfusion performed after 60 min of LAD occlusion. Mouse LV volumes and ejection fraction were obtained by echocardiography, as described previously, on the day before the surgery (baseline) and then on days 2, 7, 14, and 28 after surgery. During echocardiography, mice were maintained under light anesthesia using an inhaled mixture of 1.5% isoflurane gas and atmospheric air. The mouse was placed in a supine position on a platform with an electrical heating pad and a tensor lamp was used to provide additional heat. Mouse core body temperature was monitored with a rectal temperature probe coupled to a digital thermometer and was maintained at 37.0 ±0.2° C. ECG signals were obtained by contacting the mouse limbs, coupled with electrically conductive gel, to ECG electrodes integrated into the heating pad. The chest area was depilated to improve the quality of the B-mode echocardiographic images. Care was taken not to apply excess pressure onto the chest during scanning in order to avoid heart deformation. B-mode cardiac image sequences were acquired using a Vevo 2100 high-resolution echocardiography scanner (VisualSonics Inc., Toronto, Ontario, Canada). For each mouse, a total of 6-7 serial parasternal LV short-axis views were acquired from the apex to the LV base at 1 mm intervals. The LV cross-sectional areas were obtained by tracing the end-diastolic and end-systolic endocardial borders at each slice position. The LV volumes were then calculated as the sums of the 1 mm-thick slice volumes contoured at end-systole (ESV) or end-diastole (EDV), For wall thickening analysis, the thicknesses of the anterior and inferior walls were determined from the B-mode images and wall thickening was calculated as in an M-mode analysis. Sphericity index was calculated from long-axis B-mode images at end-diastole by dividing the length of the LV from the apex to the mitral annulus by the short axis diameter of the LV at a point two-thirds the distance from the base to the apex.

Cardiac MR imaging: In preparation for late gadolinium enhanced (LGE) cardiac MR (CMR) imaging of myocardial infarction, a length of PE-20 tubing was surgically inserted into the IP cavity and connected to a syringe preloaded with a volume of gadolinium diethylenetriamine pentaacetic acid (Gd-DTPA) contrast agent necessary to deliver a 0.1 to 0.2 mmol/kg dose. All scans were performed on a 7 Tesla small bore scanner that was equipped with a circular polarized radio frequency body coil for mice and gradient system capable of 650 mT/m maximum strength and 6667 mT/m/ms maximum slew rate (Broker, Ettlingen, Germany). All CMR was performed for three consecutive post-MI days. A multislice T2 preparation sequence for T2w edema imaging and a Tlw inversion recovery sequence for LGE infarct imaging were performed as described in [31] and [32], respectively. Localizer imaging was performed to identify double-oblique short-axis views of the LV, followed by T2w edema imaging to detect the edematous region within the entire LV. After T2w imaging, Gd-DTPA was injected for LGE infarct imaging. Ten minutes after injection, multi slice inversion recovery imaging was performed to detect the location of the infarct region within the LV myocardium.

Statistical analyses: Ail data are expressed as mean±SE. For the echocardiography study, two-way ANOVA was used to evaluate differences between and within the control group and the group treated with EcSOD vector at baseline and at serial time points after MI. Post hoc analyses (Bonferroni post-tests) were performed where appropriate. For other studies, statistical analyses were performed using Student's t-test.

### Results

### Example 1

Pharmacodynamics of transgene expression and transduction in the heart following IV administration of AAV9 postIR: In vivo bioluminescence imaging of mice that were injected IV at defined timepoints after reperfusion with the AAV9 vector expressing luciferase showed that light output was predominantly restricted to the left side of the chest cavity in all groups (see FIG. 2A for example images). A complete time course showing bioluminescence images for 2 mice from each group is included in Supplementary Data FIG. 5I. Groups that received vector after IR recorded higher light output compared to the sham-operated group at all time points (FIG. 2B, it =4 per group). When compared two days after vector administration, bioluminescence imaging showed that light output was numerically highest in the group that received vector on day 3 post-IR, followed by the groups that received vector at day 2, day 1 and 10 min post-IR (FIG. 2B). Compared to vector delivery in the sham-operated group, light output from the heart at 2 days after vector administration was elevated by 4-, 24-, 210- and 213-fold in groups injected at 10 min, 1 day, 2 days and 3 days post-IR, respectively (all comparisons p<0.05 vs. sham). The sham operated group approached steady-state levels of luciferase expression between 2-3 weeks. By contrast, luciferase expression in the groups that received vector 2 or 3 days post-IR exceeded steady-state expression levels in the sham-operated group alter less than one week.

In vitro luciferase activity assays performed on protein extracts from hearts and livers collected at the end of the study showed that in all groups (n=4 per group), luciferase activity was significantly higher in the heart compared to liver (data not shown). Compared to the sham group, luciferase activity in the heart at 7 weeks post-vector injection was 4.1-, 5.6-, 4.5- and 2.1-fold higher in the groups that received vector at 10 min, 1 day, 2 days and 3 days post-IR, respectively (FIG. 2C). Importantly, vector genome copy numbers in the heart showed trends similar to the luciferase results, with levels that were 1.8-, 2,2-, 1.8- and 1.7-fold higher in the groups that received vector at 10 min, 1 day, 2 day and 3 day post-IR, respectively compared to the sham group (FIG. 2D). These data show that ischemia and reperfusion injury to the heart, creates a more conducive environment for AAV transduction, as shown by the significantly elevated number of vector genomes present and the early and robust onset of reporter gene expression from the AAV9 vector relative to sham-operated (i.e., normal) heart.

Distribution of gene expression from AAV9 administered post-IR: The distribution of gene expression in the myocardium following vector administration 10 min post-IR (the most clinically relevant time point) was further assessed by IV injection of saline or AcTnTeGFP in sham-operated mice and in mice at 10 min post-IR (n=3 per group). Five days following IR and vector administration, eGFP expression was assessed by Western blot analysis and immunohistochemistry. Western blot analysis showed that eGFP expression (as normalized to an actin loading control) in a representative mouse that received the AAV9 vector after IR (IR+AAV9) was 3.5-fold higher compared to a sham-operated mouse injected with the same vector (Sham+AAV9, FIG. 3A). Immunohistochemistry on cryosections of the hearts confirmed no eGFP expression in the infarcted hearts of mice injected with saline (FIGS. 3B and 3B1). In sham-operated mice that received AAV9 vector (Sham+AAV9), few cardiomyocytes stained positive for eGFP expression at this early time point (FIGS. 3C and 3C1), In contrast, mice that received vector at 10 min post-IR (IR+AAV9) showed strong eGFP expression localized primarily within cardiomyocytes bordering the infarct zone (FIGS. 3D and 3D2-3D4). A lower level of eGFP expression was also noted in the remote zone of the infarcted hearts (FIG. 3D1), but this expression was nevertheless higher than that observed in sham-operated mice that received AAV9 vector (FIG. 3C1). Co-localization of a cardiac-specific marker (myoglobin) and eGFP confirmed that eGFP expression was most abundant in cardiomyocytes located at the very edge of the infarct region (FIG. 4A-C). Note that the significant levels of gene expression detected in FIGS. 3 and 4 only 5 days after vector injection represent a small fraction of the steady state gene expression levels anticipated at later time points (as demonstrated in FIG. 2).

To compare the magnitude of gene expression between the previously ischemic and remote regions of the heart, additional mice (n=4) were injected with AcTnTLuc at 10 min post-reperfusion. Five days following vector administration, hearts were explanted and luciferase activity assays were performed on tissue samples from the previously ischemic and remote regions of the hearts. Luciferase activity in the previously ischemic region was 4.3-fold higher (p<0.05) compared to the remote region of post-infarct hearts (FIG. 4D). Compared to normal myocardium in sham-operated mice, luciferase activities in the ischemic and remote regions were 15-fold (p<0.01) and 3.5-fold (p<0.05) higher, respectively (FIG. 4D).

These results show that the robust and accelerated onset of gene expression measured on day 5 following vector administration at 10 min post-IR was largely in cardiomyocytes bordering the infarct zone and also to a lesser extent in remote non-infarcted regions of the heart,

AAV9 administration after ischemia and reperfusion provides therapeutic levels of gene expression: We used an AAV9 vector carrying EcSOD under the control of the cTnT promoter (AcTnTEcSOD) to test the therapeutic benefit of AAV9 vector administration post-IR. Ten minutes post-IR, mice in the EcSOD group were injected IV with AcTnTEcSOD (n=8) while the control group (n=9) received no viral vector. Left ventricular end-diastolic volume (LVEDV) and end-systolic volume (LVESV) were measured using high-resolution echocardiography on the day before surgery (baseline) and on days 2, 7, 14 and 28 post-IR. Western blot analysis performed on hearts collected one day after the final echocardiography session indicated a 12.5-fold increase in EcSOD expression in EcSOD-treated mice over control mice after normalization for GAPDH expression (n=3 from each group, p<0.05, FIG. 5), Representative day 28 post-IR short axis echo images of control and EcSOD-treated mouse hearts at end-systole are shown in FIGS. 6A&B, Representative H&E stained tissue sections are shown in FIGS. 6C&D, illustrating the reduced chamber volumes found in EcSOD-treated hearts. Volumetric analyses between the two groups showed significant differences in relative LVEDV (p<0.05) and relative LVESV (p<0.005) as determined by two way ANOVA at days 14 and 28 post-IR (results expressed as fold changes relative to baseline). LVEDV (FIG. 6E) and LVESV (FIG. 6F) increased progressively in both control and EcSOD-treated groups with no significant differences at days 2 or 7 post-IR. In the control group, LVEDV and LVESV continued to increase through day 14 (2.2+0.2 and 5.0+0.6 fold, respectively) and day 28 (2.3+0.2 and 5.3+0.4 fold, respectively). In contrast, the EcSOD group showed no significant increases in LVEDV or LVESV after day 7 post-MI. At day 14, the EcSOD group showed a 31% reduction in relative LVEDV (p<0.05) and a 35% reduction in relative LVESV (p<0.01) compared to the control group. The significant reductions in both relative LVEDV and LVESV in the EcSOD group persisted through 28 days post-IR, yielding final reductions of 31% in relative LVEDV (p<0.05) and 35% in relative LVESV (p<0.001) as compared to the control group (FIGS. 6E and 6F). Due to the parallel changes in LVEDV and LVESV, no significant differences in LV ejection fraction (EF) were found at any time point. An analysis of sphericity index showed that the anatomic morphology of the heart was also significantly improved in the EcSOD-treated mice on day 28 post-IR relative to controls (p<0.05, FIG. 6G).

These results were supported by a wall-thickening analysis performed on the anterior (infarcled) and inferior (remote) walls of B-mode images acquired at the mid-ventricular level (Table I). This M-mode style analysis performed at 28 days post-MI revealed that the inferior wall in the EcSOD-treated group was significantly thicker at both end-diastole and end-systole than in the control group (p<0.05, both comparisons). It also detected trends towards improved wall thickening (contraction) in both the anterior and inferior walls, but these trends did not reach statistical significance. Overall, these results show that a single IV administration of AAV9 carrying AcTnTEcSOD at 10 minutes post-IR provides therapeutic levels of gene expression capable of attenuating global LV remodeling after myocardial infarction.

Demonstration that the infarct and surrounding border zone become edematous after MI: Following 60 min of coronary occlusion and reperfusion, CMR imaging was performed on days 1, 2, and 3 post-MI to delineate edematous and infarcted regions of myocardium (n>4 mice per time point). From T2w and LGE images obtained at the same short-axis slice position, it was evident that the T2w hyperintense edematous region and LGE infarct region showed good spatial correspondence (FIG. 7). T2w hyperintense signals were strongest on day 2 post-MI. In all mice at all days, the infarct region was consistently confined within the edematous region, and the size of the infarct region was smaller than the edematous region. These results show that the border zone immediately surrounding the infarct region becomes significantly edematous shortly after MI.

Additionally, an AAV9 vector has been prepared and used in combination with Examples 1-3 to knock-down transgenic eGFP gene expression in the heart (data not shown).

TABLE 1
Example 1
Table I: Wall thickening analysis of Control vs. EcSOD-treated groups

| | End-Diastolic WT (mm) | | End-Systolic WT (mm) | | Wall Thickening (%) | |
|---|---|---|---|---|---|---|
| | Control | EcSOD | Control | EcSOD | Control | EcSOD |
| Anterior | 0.31 ± 0.03 | 0.33 ± 0.03 | 0.29 ± 0.03 | 0.33 ± 0.03 | −7.1 ± 2.9% | 2.6 ± 5.9% |
| Inferior | 0.46 ± 0.03 | 0.57 ± 0.04* | 0.55 ± 0.03 | 0.72 ± 0.05** | 20.2 ± 4.3% | 27.8 ± 5.5% |

### Discussion

### Example 1

In the current study, we demonstrate that: 1) the onset of AAV9-mediated gene expression is accelerated when the vector is delivered after IR injury; 2) this enhanced expression is most pronounced in cardiomyocytes bordering the infarct region; 3) systemic administration ten minutes post-IR of an AAV9 vector expressing EcSOD significantly inhibits global LV remodeling subsequent to MI; and 4) the border zone becomes edematous shortly after MI, consistent with a localized increase in vascular permeability.

As shown in our previous work, AAV9-mediated gene expression can be effectively restricted to cardiomyocytes using the cardiac-specific cTnT promoter [25]. Using the AAV9 capsid in combination with the cTnT promoter, we showed that eGFP expression after systemic administration was virtually undetectable in both vascular smooth muscle and endothelial cells in the heart, even while it was expressed in >95% of cardiomyocytes. Despite being the most efficient gene delivery platform currently available for cardiomyocytes, gene expression from AAV9 does not approach full strength in the normal heart until 2-3 weeks after vector administration (see sham in FIG. 2B). Conventional vectors packaged in the AAV2 capsid have shown an even more prolonged lag phase, taking up to 8 weeks to reach a steady-state plateau of gene expression in the heart. In order to accommodate this limitation, previous studies of AAV-mediated gene therapy for MI have typically employed a preemptive gene therapy approach in which AAV2 vectors carrying therapeutic genes were directly injected into the LV wall 4-6 weeks before the induction of myocardial ischemia. In a similarly designed study of preemptive delivery, our laboratory recently demonstrated that a single direct intramuscular injection into the LV wall of an AAV9 vector expressing EcSOD from the cTnT promoter four weeks before the induction of MI caused a 22-fold increase in EcSOD activity which significantly decreased infarct size.

The delay in reaching maximal gene expression in normal myocardium may also explain why only a few previous studies have attempted to protect the heart against LV remodeling by delivering AAV vectors after MI has already occurred. This delay is especially problematic in mice, where global LV remodeling starts within a day after reperfused MI and nears completion within 2 weeks. Therefore, an early onset of therapeutic gene expression following vector administration is important in curtailing LV remodeling, particularly in mouse models of ML Nevertheless, a few previous studies have explored the utility of administering AAV by direct injection into the LV wall after ischemia/reperfusion injury. Su et al. directly injected an AAV1 vector carrying VEGF driven by a cardiac specific promoter into mouse myocardium after MI. Jaequier et al. and Saeed et al. directly injected AAV2 vectors carrying VEGF cDNA into swine myocardium after MI. Despite the prolonged lag phase to full gene expression documented in normal hearts, AAV2-mediated VEGF gene delivery after MI brought about significant improvements in cardiac function. However, none of these previous studies employed systemic administration, nor did they report the phenomenon of preferential transduction and early onset of gene expression in cardiomyocytes located in the infarct border zone. The results of the current study demonstrate that systemic administration of an AAV9 vector following ischemia/reperfusion injury provides for robust and early onset gene expression, particularly in the cardiomyocytes at risk bordering the infarct region. Since this is the first report documenting the phenomenon of preferential transduction of cardiomyocytes at risk following systemic administration of AAV vector after IR injury, it may warrant further investigation using other serotypes of AAV and in larger animal models of IR injury. The current study suggests that AAV9 vectors may have considerable potential to deliver therapeutic genes to the infarct border zone after ML providing a means to genetically reprogram the subsequent LV remodeling process and the potential to avert heart failure in patients who survive a large ML

Myocardial IR injury increases capillary permeability, both as a direct result of ischemia and as the indirect result of the local release of inflammatory mediators upon reperfusion. The increase in vascular permeability allows greater fluid passage into the extravascular space, disrupting the normal balance between capillary filtration and lymphatic reabsorption, resulting in the accumulation of fluid in the extravascular space (edema). The CMR experiments summarized in FIG. 7 confirm that edema develops quickly in the infarct border zone in this mouse model of reperfused myocardial infarction, providing evidence of increased capillary permeability in the border zone and suggesting a potential mechanism by which AAV9 vectors circulating in the bloodstream might gain enhanced access to viable cardiomyocytes within the edematous region. This mechanism of enhanced access to cardiomyocytes is supported by the fact that ischemia-induced elevations in gene expression (FIGS. 2B&C) are accompanied by similar increases in the numbers of viral genomes/μg of genomic DNA (FIG. 2D).

The various serotypes of AAV accomplish transduction by first binding to different cell surface receptors. AAV2 uses heparin sulfate proteoglycan, FGFR1 and αvβ5 integrin, AAV1 and 6 use α2,3 and α2,6 N-linked sialic acids, while AAV 8 and 9 use the 36/37 kDa laminin receptor [40], Recently, Shea et al. showed that desialylated N-linked glycans with terminal galactosyl residues also serve as receptors for AAV9 [41]. Given that sialidase activity and free sialic acid are significantly increased in the plasma from patients with ischemia (Hanson et. al., 1987, Am. Heart J., 114:59), it is plausible that endogenous siaiidases are locally activated by ischemia and their activation may “unmask” receptors for AAV9 through the desialylation of N-linked glycans. Note that this potential mechanism for the ischemic enhancement of AAV9-mediated transduction may act in synergy with the mechanism of increased vascular permeability implicated here (FIG. 7), since the mechanism of receptor “unmasking” by endogenous sialidases can only be realized after AAV9 escapes the bloodstream, a process that is enhanced by increased vascular permeability.

Following receptor binding and viral entry into the cell, capsid uncoating and second strand DNA synthesis are the rate limiting steps for gene expression from the AAV genome. Previous studies have shown that the reagents that induce DNA damage and repair activity, such as hydroxyurea, UV irradiation, and topoisomerase inhibitors, accelerate the onset of gene expression from AAV2 vectors [9, 19, 42, 43]. Recently, it was shown that another stress inducing factor, prolonged fasting, significantly improves AAV transduction in skeletal muscle, heart and liver following systemic administration of AAV2, 6 and 9 vectors. The DNA damage that results from IR injury may well be a contributing mechanism for the observed increase in transduction efficiency, because DNA damage causes rapid relocalization of the heterotrimeric DNA repair complex consisting of Mre11, Rad50 and Nbs1 (MRN) to the site of DNA damage. Furthermore, degradation or re-localization of the MRN complex to sites of DNA damage appears to create a nuclear environment that is more conducive for AAV-mediated gene expression. Collectively, these studies suggest that rapid relocalization of the MRN DNA repair complex due to IR injury might be another mechanism contributing to the enhanced transduction of “at risk” cardiomyocytes in the border zone after MI.

The results of this study have implications for both basic science and clinical translation. From the perspective of basic cardiovascular science, the ability to selectively target gene expression to the infarct border zone after MI opens the possibility of examining the function of gene expression (or knockdown via siRNA) in a tissue-, region- and time-selective manner after MI. From the clinical perspective, the current study suggests the possibility of genetically reprogramming gene expression in the infarct border zone by simple IV administration after MI. In this manner, gene therapy protocols could be used in combination with conventional pharmacologic interventions or even cell-based therapies to improve long-term outcomes after MI.

### Bibliography

### Example 1

1. Cohn J N, Ferrari R, Sharpe N Cardiac remodeling-concepts and clinical implications: a consensus paper from an international forum on cardiac remodeling. On behalf of an international forum on cardiac remodeling. J Am Coll Cardiol 2000; 35: 569-582
2. Ross A J, Yang Z, Berr S S, et al. Serial MRI evaluation of cardiac structure and function in mice after reperfused myocardial infarction. Magnetic Resonance in Medicine 2002; 47: 1158-1168
3. Yang Y, Nunes F A, Berencsi K, et al. Cellular immunity to viral antigens limits El-deleted adenoviruses for gene therapy. Proceedings of the National Academy of Sciences 1994; 91: 4407-4411
4. Dash R, Azab B, Shen X-N, et al. Developing an effective gene therapy for prostate cancer: New technologies with potential to translate from the laboratory into the clinic. Discovery Medicine 2011; 11: 46-56
5. Kang E, Yun C-O Current advances in adenovirus nanocomplexes: more specificity and less immunogenicity. BMB reports 2010; 43: 781-788
6. Hernandez Yj, Wang J, Kearns W G, et al. Latent Adeno-Associated Virus Infection Elicits Humoral but Not Cell-Mediated Immune Responses in a Nonhuman Primate Model. Journal of Virology 1999; 73: 8549-8558.
7. Gao G, Vandenberghe L H, Alvira M R, et al. Clades of Adeno-associated viruses are widely disseminated in human tissues. Journal of Virology 2004; 78: 6381-6388
8. Riitledge E A, Halbert C L, Russell D W Infectious clones and vectors derived from adeno-associated virus (AAV) serotypes other than AAV type 2. Journal of Virology 1998; 72: 309-319
9. Du L, Kido M, Lee D V, et al. Differential myocardial gene delivery by recombinant serotype-specific adeno-associated viral vectors. Molecular Therapy: the Journal of the American Society of Gene Therapy 2004; 10: 604-608
10. Palomeque J, Chemaly E R, Colosi P, et al. Efficiency of eight different AAV serotypes in transducing rat myocardium in vivo. Gene Therapy 2007; 14: 989-997
11. Zincarelli C, Soltys S, Rengo G, et al. Analysis of AAV Serotypes 1-9 Mediated Gene Expression and Tropism in Mice After Systemic Injection. Molecular Therapy 2008; 16: 1073-1080
12. Inagaki K, Fuess S, Storm T A, et al. Robust systemic transduction with AAV9 vectors in mice: efficient global cardiac gene transfer superior to that of AAV8. Molecular Therapy 2006; 14: 45-53
13. Bostick B, Ghosh A, Yue Y, et al. Systemic AAV-9 transduction in mice is influenced by animal age but not by the route of administration. Gene Therapy 2007; 14: 1605-1609
14. Gregorevic P, Blankinship M J, Allen J M, et al. Systemic delivery of genes to striated muscles using adeno-associated viral vectors. Nature Medicine 2004; 10: 828-834
15. Townsend D, Blankinship M J, Allen J M, et al. Systemic administration of micro-dystrophin restores cardiac geometry and prevents dobutamine-induced cardiac pump failure. Molecular Therapy 2007; 15: 1086-1092
16. Pacak C A, Mali C S, Thattaliyath B D, et al. Recombinant adeno-associated virus serotype 9 leads to preferential cardiac transduction in vivo. Circulation Research 2006; 99: e3-9
17. Cataliotti A, Tonne J M, Beliavia D, et al. Long-Term Cardiac pro-B-Type Natriuretic Peptide Gene Delivery Prevents the Development of Hypertensive Heart Disease in Spontaneously Hypertensive Rats/Clinical Perspective. Circulation 2011; 123: 1297-1305
18. Kaya Z, Leib C, Werfel S, et al. Comparison of IL-10 and MCP-1-7ND gene transfer with AAV9 vectors for protection from murine autoimmune myocarditis. Cardiovascular Research 2011; 91: 116-123
19. Prasad K-M R, Xu Y, Yang Z, et al. Topoisomerase inhibition accelerates gene expression after adeno-associated virus-mediated gene transfer to the mammalian heart. Molecular Therapy 2007; 15: 764-771
20. Thomas C E, Storm T A, Huang Z, et al. Rapid uncoating of vector genomes is the key to efficient liver transduction with pseudotyped adeno-associated virus vectors. Journal of Virology 2004; 78: 3110-3122
21. Agrawal R S, Muangman S, Layne M D, et al. Pre-emptive gene therapy using recombinant adeno-associated virus delivery of extracellular superoxide dismutase protects heart against ischemic reperfusion injury, improves ventricular function and prolongs survival. Gene Ther 2004; 11: 962-969
22. Pachori A S, Melo L G, Zhang L, et al. Chronic recurrent myocardial ischemic injury is significantly attenuated by pre-emptive adeno-associated virus heme oxygenase-1 gene delivery. J Am Coll Cardiol 2006; 47: 635-643
23. Jacquier A, Higgins C B, Martin A J, et al. Injection of Adeno-associated Viral Vector-Encoding Vascular Endothelial Growth Factor Gene in Infarcted Swine Myocardium: MR Measurements of Left Ventricular Function and Strain. Radiology 2007; 245: 196-205
24. Saeed M, Saloner D, Martin A, et al. Adeno-associated Viral Vector-Encoding Vascular Endothelial Growth Factor Gene: Effect on Cardiovascular MR Perfusion and Infarct Resorption Measurements in Swine. Radiology 2007; 243: 451-460
25. Prasad K-M R, Xu Y, Yang Z, et al. Robust Cardiomyocyte-Specific Gene Expression Following Systemic Injection of AAV: In Vivo Gene Delivery Follows a Poisson Distribution. Gene Therapy 2011; 18: 43-52
26. Prasad K-M R, Smith R S, Xu Y, et al. A single direct injection into the left ventricular wall of an adeno-associated virus 9 (AAV9) vector expressing extracellular superoxide dismutase from the cardiac troponin-T promoter protects mice against myocardial infarction. J Gene Med 2011; 13: 333-341
27. Ried M U, Girod A, Leike K, et al. Adeno-associated virus capsids displaying immunoglobulin-binding domains permit antibody-mediated vector retargeting to specific cell surface receptors. Journal of Virology 2002; 76: 4559-4566
28. Yang Z, Berr S S, Gilson W D, et al. Simultaneous evaluation of infarct size and cardiac function in intact mice by contrast-enhanced cardiac magnetic resonance imaging reveals contractile dysfunction in noninfarcted regions early after myocardial infarction. Circulation 2004; 109: 1161-1167
29. Wu J C, Inubushi M, Sundaresan G, et al. Optical imaging of cardiac reporter gene expression in living rats. Circulation 2002; 105: 1631-1634.
30. Li Y, Garson C D, Xu Y, et al. High frequency ultrasound imaging detects cardiac dyssynchrony in noninfarcted regions of the murine left ventricle late after reperfused myocardial infarction. Ultrasound Med Biol 2008; 34: 1063-1075
31. Beyers R J, Smith R S, Xu Y, et al. T2-weighted MRI of post-infarct myocardial edema in mice. Magnetic Resonance in Medicine 2011; 67: 201-209
32. Helm P A, Caravan P, French B A, et al. Postinfarction Myocardial Scarring in Mice: Molecular MR Imaging with Use of a Collagen-targeting Contrast Agent. Radiology 2008; 247: 788-796
33. Su H, Huang Y, Takagawa J, et al. AAV serotype-1 mediates early onset of gene expression in mouse hearts and results in better therapeutic effect. Gene Therapy 2006; 13: 1495-1502
34. Khandoga A G, Khandoga A, Anders H-J, et al. Postischemic vascular permeability requires both TLR-2 and TLR-4, but only TLR-2 mediates the transendotbelial migration of leukocytes. Shock 2009; 31: 592-598
35. Weis S M Vascular permeability in cardiovascular disease and cancer. Current Opinion in Hematology 2008; 15: 243-249
36. Summerford C, Samuiski R J Membrane-associated heparan sulfate proteoglycan is a receptor for adeno-associated virus type 2 virions. Journal of Virology 1998; 72: 1438-1445

37. Summerford C, Barflett I S, Samuiski R J Alpb.aVbet.a5 integrin: a co-receptor for adeno-associated virus type 2 infection. Nature Medicine 1999; 5: 78-82
38. Qing K, Mali C, Hansen J, et al. Human fibroblast growth factor receptor 1 is a co-receptor for infection by adeno-associated virus 2. Nature Medicine 1999; 5: 71-77
39. Wu Z, Miller E, Agbandje-McKenna M, et al. Alpha-2,3 and alpha-2,6 N-Linked Sialic Acids Facilitate Efficient Binding and Transduction by Adeno-Associated Virus Types 1 and 6. Journal of Virology 2006; 80: 9093-9103
40. Akache B, Grimm D, Pandey K, et al. The 37/67-kilodalton laminin receptor is a receptor for adeno-associated virus serotypes 8, 2, 3, and 9. J Virol 2006; 80:9831-9836
41. Shen S, Bryant K D, Brown S M, et al. Terminal N-Linked Galactose Is the Primary Receptor for Adeno-associated Virus 9. Journal of Biological Chemistry 2011; 286: 13532-13540
42. Alexander I E, Russell D W, Spence A M, et al. Effects of gamma irradiation on the transduction of dividing and nondividing cells in brain and muscle of rats by adeno-associated virus vectors. Hum Gene Ther 1996; 7: 841-850
43. Russell D W, Alexander I E, Miller A D DNA synthesis and topoisomerase inhibitors increase transduction by adeno-associated virus vectors. Proc Natl Acad Sci USA 1995; 92:5719-5723
44. Moulay G, Scherman D, Kidder A Fasting Increases the In Vivo Gene Delivery of AAV Vectors. Clin Transl Sci 2010; 3: 333-336
45. Maser R S, Monsen K J, Nelms B E, et al. hMre11 and hRad50 nuclear foci are induced during the normal cellular response to DNA double-strand breaks. Mol Cell Biol 1997; 17: 6087-6096
46. Mirzoeva O K, Petrini J H DNA damage-dependent nuclear dynamics of the Mre11 complex. Mol Cell Biol 2001; 21: 281-288
47. Schwartz R A, Palacios J A, Cassell G D, et al. The Mre11/Rad50/Nbs1 Complex Limits Adeno-Associated Virus Transduction and Replication. Journal of Virology 2007; 81: 12936-12945
48. Souza et al. (U.S. Pat. Pub. No. 2011/0212529, published Sep. 1, 2011).
49. Ordahl et al. (U.S. Pat. No. 5,266,488)
50. Hanson et al., 1987, Am. Heart J., 114:59
51. Saqib et al, J. Vase. Surg., 2011, 54:3:810, Epub 2011 Jul. 2, AAV9-Mediated Overexpression of Extracellular Superoxide Dismutase Improves Recovery from Surgical Hind-limb Ischemia in BALB/c Mice.

### Example 2

### AAV9 Efficiently Targets Ischemic Skeletal Muscle Following Systemic Delivery

Materials and Methods

Plasmids: The AAV vectors bearing the CMV promoter driving the expression of firefly luciferase (AAV/CMV/Luc) or eGFP (AAV/CMV/eGFP) have been described previously (Example 2, FIG. 1). Construction of AAV vector bearing the CK6 promoter driving the expression of firefly luciferase (AAV/CK6/Luc) or eGFP (AAV/CK6/eGFP) was accomplished in two steps. First, the CMV promoter was removed from AAV/CMV/Luc and AAV/CMV/eGFP by double digestion with XbaI and HindIII. Second, a PGR amplified 571 bp CK6 MCK enhancer/promoter was directionally inserted as an XbaI-HindIII fragment (Hauser et al., 2000, see for example, their FIGS. 1 and 2 and also the present Example 3 and its figures Example 3, FIG. 1 and Example 3, FIG. 2). They began with an MCK genomic fragment having GenBank Accession No. AF188002. Their CK6 contained the 2RS5 enhancer (206 bp SEQ ID NO:15 herein) and a proximal promoter extending from nucleotide position −358 to +7 bp relative to the transcriptional start site, yielding a 571 bp enhancer/promoter construct. The 571 bp muscle specific CK6 MCK enhancer/promoter construct was a kind gift of Dr. S. D. Hauschka. The AAV-9 pseudotyped AAV.MCK6.eGFP.bGH vector used for the neuraminidase experiments was obtained from the Penn Vector Core in the School of Medicine Gene Therapy Program at the University of Pennsylvania.

AAV vector production: AAV vectors were packaged in HEK 293 cells by the double or triple transfection method, and then purified by ammonium sulfate fractionation and iodixanol gradient centrifugation as described previously. Titers of the AAV vectors (viral genomes/ml) were determined by quantitative real-time PCR as described previously.

Animal procedures: Animal protocols used in this study were approved by the Institutional Animal Care and Use Committee and conformed to the “Guide for the Care and Use of Laboratory Animals” (NIH Publication 85-23, revised 1985). All mice (C57BL/6 and BALB/c) (15-20 weeks old) were purchased from The Jackson Laboratories (Bar Harbor, Me.). Age-matched (15-20 week old) male mice were used for all the experiments to exclude estrogen as a potential confound in the HLI model described below.

Induction of hindlimb ischemia (HLI): Mice underwent unilateral femoral artery ligation and excision on the left hindlimb as described previously. Necrosis was visually assessed each day. Blood flow in the ischemic and contralateral non-ischemic limbs was measured as described previously with a laser Doppler perfusion imaging system (Perimed, Stockholm, Sweden).

AAV vector delivery: For intravenous (IV) injection, mice were anesthetized with isoflurane as described above and the AAV-9 solutions (50-100 μl containing 4.15×10¹¹ viral genomes) were slowly injected via the right jugular vein on the 7-8th day following HLI surgery. For the neuraminidase (NAD) experiments, 100 μl containing 4.24×10¹¹ viral genomes were injected via the right jugular vein 2-4 hours following intramuscular injection of NAD into the left tibialis anterior muscles. The AAV vectors can also be administered systemically (parenterally or enterally) or locally.

Bioluminescence imaging in vivo: Bioluminescence imaging was performed using an IVIS 100 system (Caliper Life Sciences, Hopkinton, Mass.). Luciferase expression in live mice was non-invasively detected after the IP injection of luciferin and images were processed as described previously. Equal-sized regions of interest (ROIs) were marked over each hindlimb and upper abdomen to obtain estimates of bioluminescence intensity.

Quantitative in vitro luciferase activity assays: Luciferase activity was measured using luciferase assay reagents from Promega Corp. (Madison, Wis.). After bioluminescence imaging and euthanasia at 10-14 days post vector injection; the heart, liver, and skeletal muscles were collected from experimental mice. Protein extracts were prepared and luciferase activities (Relative light units, RLU) were determined using a FLUOstar Optima micro-plate reader (BMG Labtech, Durham, N.C.).

Fluorescence imaging: eGFP expression and desialylation of cell surface glycans in mouse tissues were documented by fluorescence microscopy using a Zeiss LSM 700 confocal microscope (Gottingen, Germany). For eGFP expression fourteen days following vector administration, animals were euthanized for muscle collection and fixation in 3.7% paraformaldehyde at 4° C. for 1 hour. After (3×) 5 min PBS washes, tissues were equilibrated with 30% sucrose in PBS overnight. Fifteen μm thick cryosections were then cut and used for documenting eGFP expression.

For assessing sialylated and desialylated cell surface glycans, animals were euthanized 7 days post-HLI. Ischemic and contralateral muscles were harvested and placed in OCT for snap freezing in liquid nitrogen. Seven μm cryostat sections were prepared to assess the differential distribution of sialylated or desialylated glycans in ischemic versus non-ischemic muscles. Staining was performed using the biotinylated lectins, Maackia amurensis lectin (MAL I) and Erythrina cristagalli lectin (ECL) (Vector Laboratories, Burlingame, Calif.). Lectins were visualized using Streptavidin-Alexa Fluor-555 (Invitrogen Carlsbad Calif.). Muscle actin was detected using FITC-conjugated, mouse monoclonal anti-actin antibody clone AC40 (Sigma Chemicals St. Louis, Mo.).

Western blot: For quantitation of eGFP expression in muscles with sialylated versus desialylated cell surface glycans, animals were pre-treated with intramuscular injection of neuraminidase from V. cholerae (Sigma-Aldrich, St. Louis, Mo.) into their left tibialis anterior (TA) muscles (2 miliiunits/TA) with contralateral TA muscles serving as negative controls. Two to four hours later, all of these animals received the vector intravenous iy.

Fourteen days following the vector administration, the animals were euthanized, muscles harvested, and protein extracts prepared. The muscle homogenates were then separated on polyacryiamide gels, transferred to PVDF membranes, blocked and blotted with antibodies against eGFP and actin. eGFP protein expression was normalized against actin expression for quantitative analysis.

Statistical analysis: Data were expressed as mean±SEM. For statistical comparisons of gene expression, luciferase activities in the various tissues were compared using 1-way ANOVA. Western blot densitometry comparisons were performed by t-test. P<0.05 was considered statistically significant in all of the comparisons.

Determination of AAV vector genome copy number per μg genomic DNA: The AAV genomic backbone AAV/CK6/Luc was cross-packaged into capsids from AAV serotypes 9 and 1 for injection as described above. Two weeks after vector administration, total genomic DNA from a panel of tissues was prepared using QIAamp DNA minikits (Qiagen, Inc). Real-time qPCR using S YBR Green I detection was performed on a BioRad iCycler (Hercules, Calif., USA). The following primers were used for amplifying the firefly luciferase gene: SEQ ID NO: 5-5′-AGAACTGCCTGCGTGAGATT-3′ (forward) and SEQ ID NO:6-5′-AAAACCGTGATGGAATGGAA-3′ (reverse). Known copy numbers (103-108) of the plasmid AAV/CK6/Luc were used to construct the standard curve. The results were expressed as mean AAV vector genome copy numbers per μg of genomic DNA.

Statistical analysis: Data were expressed as mean±SEM. For statistical comparisons of gene expression, luciferase activities in the various tissues were compared using 1-way ANOVA. For Western blot densitometry comparisons, statistical analysis was performed with paired t-test. P0.05 was considered statistically significant in all of the comparisons.

### Results

### Example 2

### Magnitude and Specificity of Gene Expression from Intravenous Injection of AAV-9 Harboring the CMV Promoter

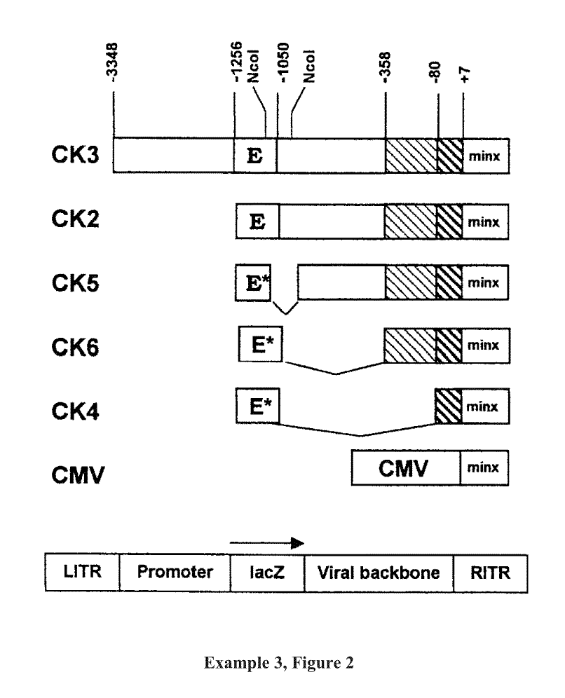

The perfusion ratio of ischemic to non-ischemic hindlimbs in C57Bl/6 mice (n=5) immediately post-HLI was 0.34±0.12 (mean±SEM), As anticipated, the perfusion ratio recovered partially to 0.48±14 by post-operative (post-op) day 7, at which time the mice received IV injections of AAV/CMV/Luc (4.15×10¹¹ viral genomes (vg)/animal) via the right internal jugular vein. Luciferase expression was then monitored by non-invasive in vivo bioluminescence imaging. Age-matched C57Bl/6 male mice that did not undergo HLI and did not receive any vector served as negative controls (Example 2, FIG. 2a). As indicated by the blue color-coding, luciferase expression from the CMV promoter was observed throughout the body on post-AAV days 7 (Example 2, FIG. 2b) and 14 (Example 2, FIG. 2c). However, luciferase expression appeared strongest in the upper abdominal region corresponding to liver. Interestingly, on both post-AAV days 7 and 14, despite expression being driven by the CMV promoter, the bioluminescence signals appeared stronger in the ischemic hindlimbs (mouse's left side, rightmost hindlimb in Example 2, FIG. 2) when compared to the non-ischemic, contralateral hindlimb. ROI analysis was then used to estimate relative luciferase signal intensity in each hindlimb and the upper abdomen (corresponding to liver). On post-AAV day 7, the mean bioluminescence signal from ischemic hindlimbs was 2.7±0.3-fold higher than the non-ischemic limbs and 17.7±0.8-fold lower than in the liver (Example 2, FIG. 2d). On post-AAV day 14, bioluminescence in the ischemic limbs was 4.3±0.4-fold higher than in the non-ischemic limbs and 4.5±0.4-fold lower than in the liver.

While bioluminescence imaging provides a non-invasive estimate of relative luciferase activities in serial studies, it is difficult to compare values between tissues due to differences in tissue depth and the differential absorption of photons by different tissues. For this reason, rigorous quantitative measurement of luciferase activity was performed in tissue extracts from the various organs as shown in Example 2, FIG. 2e. Luciferase activity in the ischemic gastrocnemius (GA) muscles of mice treated with AAV/CMV/Luc was 10.5±0.6 fold higher than in the contralateral GA, 2.0±0.3-fold higher than in the heart, and 1.8±0.3-fold higher than in the liver. These results demonstrate that AAV-9 is highly effective for delivering gene(s) to ischemic skeletal muscle following systemic delivery, even when using a promiscuous (non-tissue-specific) promoter.

Magnitude and Specificity of Gene Expression from Intravenous Injection of AAV-9 Harboring the CK6 Promoter

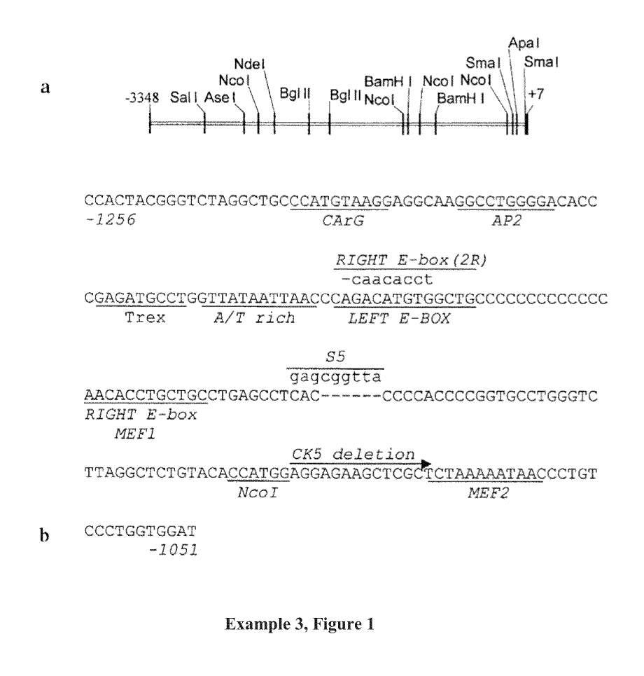

HLI was surgically induced in left hindlimbs of adult C57Bl/6 mice (n=4). Immediately after surgery on post-op day 0, the ratio of perfusion as measured by laser Doppier between ischemic and non-ischemic hindlimbs was 0.34±0.12 (Mean±SEM), On post-op day 7, the perfusion ratio had partially recovered to 0.48±35. On post-op day 8, all mice received IV injections of AAV/CK6/Luc (4.15×10¹¹ viral genomes (vg)/animai) via the right internal jugular vein. Luciferase expression was again monitored by bioluminescence imaging. Bioluminescence signals appeared strongest in the ischemic hindlimbs on post-AAV days 6 (Example 2, FIG. 2f) and 10 (Example 2, FIG. 2g). Using ROI analysis, the mean bioluminescence signal in the ischemic limbs was 50.5±1.8-fold higher than non-ischemic limbs and 17.2±1.0-fold higher than liver on day 6 post-AAV (Example 2, FIG. 2h). On day 10 post-AAV, bioluminescence in ischemic limbs was 37.8±1.8-fold higher than non-ischemic limbs and 9.8±0.8-fold higher than the liver (Example 2, FIG. 2h). Similar results were obtained in parallel experiments performed in BALB/c mice (data not shown).

The more rigorous, quantitative measurement of luciferase activity in tissue extracts from selected organs is presented in Example 2, FIG. 2i. Again, luciferase activity was significantly higher in the ischemic hindlimb muscles compared to contralateral non-ischemic muscles or liver. Luciferase activity in the ischemic GA muscle of mice treated with AAV/CK6/Luc was 34.1±1.5-fold higher than in the contralateral GA, 28.1±1.3-fold higher than in the heart, and 150.2±3.1-fold higher than in the liver (all comparisons p<0.05). Luciferase activity in the non-ischemic GA was 1.2±0.3-fold higher than in the heart and 6.6±0.6-fold higher than in the liver. Furthermore, luciferase activity in the ischemic GA was 1.9-fold higher while that in the liver was 41.7-fold lower in the CK6 group when compared to the CMV group. These results clearly demonstrate that the combination of AAV-9 and the CK6 promoter is highly efficient and selective for delivering genes to ischemic skeletal muscle following systemic delivery.

Distribution of eGFP expression in ischemic hindlimb muscle confirms the efficiency of AAV-9: Vectors carrying the enhanced green fluorescence protein (eGFP) gene driven by the CMV or CK6 promoters (AAV/CMV/eGFP and AAV/CK6/eGFP) were systemically administered to adult C57Bl/6 mice (n=5 for CMV and n=2 for CK6) via jugular vein at a dose of 4.15×10¹¹ vg per mouse on the 7th day following HLI surgery. Two weeks following vector injection, eGFP expression in the mouse hindlimb skeletal muscles was assessed by fluorescence microscopy (Example 2, FIG. 3). The results show that the AAV-9 capsid together with the CK6 promoter transduces ischemic skeletal myofibers (Example 2, FIG. 3b) far more efficiently than the non-ischemic ones (FIG. 3a) after systemic delivery. Microscopic analysis of >3000 myofibers taken from different sections of ischemic tibialis anterior (TA) muscles from mice treated with AAV/CK6/eGFP revealed that 50-55% of the myofibers exhibited fluorescent signal above background auto fluorescence. (Example 2, FIG. 3b). In contrast, the expression of eGFP in the skeletal myofibers following systemic delivery was relatively low when gene expression was driven by the CMV promoter (Example 2, FIGS. 3c, d). With the CMV promoter, <0.5% of the myofibers in the ischemic TA exhibited fluorescent signal above background. Thus the transduction rate for AAV-9 in ischemic skeletal myofibers is significantly higher with the CK6 than with the CMV promoter after IV injection in adult mice. These results using an independent reporter system confirm that the combination of AAV-9 capsid and CK6 promoter is highly-efficient for the selective delivery of gene(s) to ischemic skeletal muscles following systemic delivery.

HLI induces marked desialylation of cell surface N-linked glycans, thereby unmasking the primary receptor for AAV-9 binding: HLI was surgically induced in the left hindlimbs of adult male BALB/c mice (n=3). Seven days following HLI, the distribution of sialylated versus desialyiated cell surface glycans in mouse hindlimb skeletal muscles was assessed by fluorescence microscopy using lectin staining. Of the two lectins used, MAL I binds to a2,3-sialylaled glycans whereas ECL binds to the desialyiated galactose residues of cell surface glycans. Myofibers from the ischemic TA showed abundant ECL staining along the cell surface compared to a weaker staining seen in the non-ischemic TA muscles (Example 2, FIG. 4a, top). Conversely, the distribution of MAL I was abundant in non-ischemic TA muscles and was very weak in ischemic TA muscles (Example 2, FIG. 4a, bottom). Similar results were seen in C57Bl/6 mice (data not shown). These results demonstrate that the induction of hindlimb ischemia causes marked desialylation of cell surface N-linked glycans, thus unmasking the primary cell surface attachment factor for AAV-9.

Neuraminidase pretreatment increases gene expression following intravenous injection of AAV-9 harboring the CK6 promoter, in the absence of hindlimb ischemia: Neuraminidase (NAD) was injected IM into the left tibialis anterior (TA) muscles of adult male C57Bl/6 mice (n=9). Two to four hours later, all of these mice received intravenous injections of the AAV.MCK6.eGFP.bGH vector via jugular vein at a dose of 4.24×1011 vg per mouse. Fourteen days following the vector administration, animals were euthanized, and eGFP protein expression was assessed using Western blot analysis (Example 2, FIG. 4b). eGFP expression was 1.95-fold higher in the NAD-treated TA as compared to the contralateral TA (Example 2, FIG. 4c, p<0.05).

Magnitude of Gene Expression and Tropism of Tissue Distribution Following Intravenous Injection of AAV-1 and 9 Harboring the CK6 Promoter Farther Implicates Hindlimb Ischemia in the Unmasking of Cell Surface Receptors, Thereby Facilitating Selective Transduction by AAV-9

HLI was surgically induced in left hindlimbs of adult C57Bl/6 mice (n=5 per group) 7 days prior to the injection of AAV/CK6/Luc genomes packaged in either AAV-9 or AAV-1 capsids. On post-op day 7, the ratio of perfusion in ischemic vs. non-ischemic hindlimbs as measured by laser Doppler was 0.44±0.13 (Mean±SEM) for the AAV-9 group and 0.29±11 for the AAV-1 group. After laser Doppler measurement on post-op day 7, 5 mice received IV injections of AAV-9/CK6/Luc (4.15×1011 viral genomes (vg)/animal) via the right internal jugular vein, while the remaining 5 mice were similarly treated with AAV-1/CK6/Luc. Luciferase expression was again monitored by bioluminescence imaging.

In the AAV-9 group, bioluminescence signals again appeared strongest in the ischemic hindlimbs on post-AAV days 7 (Example 2, FIG. 5a) and 0.14 (Example 2, FIG. 5b) with markedly reduced expression in liver and little luciferase expression, if any, detected elsewhere. In the AAV-1 group, bioluminescence signal intensities in ischemic hindlimbs were 150-250 fold less than that seen in mice injected with the same vector genome packaged in AAV-9 capsids (FIG. 5c-d). Furthermore, the marginal bioluminescence signal from ischemic hindlimbs was no stronger than that generated by liver.

The more rigorous, quantitative measurement of luciferase activity in tissue extracts from selected organs is presented in FIG. 5e. Again, luciferase activity in the AAV-9 group was significantly higher in ischemic hindlimb muscle compared to contralateral non-ischemic muscle, liver, or other organs. Luciferase activity in the ischemic muscle of mice treated with AAV9/CK6/Luc was >44-fold higher than in the non-ischemic muscle or liver, >136-fold higher than in the heart and >2000-fold higher than in the brain. Furthermore, luciferase activity in ischemic muscle was 24-fold higher in the AAV-9 group as compared to the AAV-1 group (all comparisons p<0.05 by ANOVA).

We next compared the viral genome (vg) copy numbers persisting in tissue samples at 14 days post-AAV injection, using qPCR (FIG. 5f), Interestingly, AAV-9 vg copies were significantly higher in the liver (3.3×10⁷ vg copies/tig host genomic DNA) than in any other tissue examined (all comparisons p<0.05 by ANOVA). Nevertheless, the next highest concentration of vector genomes was found in ischemic muscle (1.7×10⁵ vg copies/μg host genomic DNA) followed by kidney, brain, non-ischemic muscle and heart. In contrast, AAV-1 did not exceed 3×10⁵ vector genome (vg) copy numbers per μg host genomic DNA in any tissue examined; with the highest copy numbers found in the liver, followed by non-ischemic muscle, brain, ischemic muscle, kidney and heart. Finally, the vg/μg genomic DNA copy numbers for AAV-9 were 5.6-fold higher in ischemic vs. non-ischemic muscle; whereas this trend was reversed for AAV-1 which had 6.0-fold higher copy numbers in non-ischemic vs. ischemic muscle.

These results clearly demonstrate that AAV-9 selectively targets ischemic hindlimb muscle, and that the AAV serotype 9 capsid, in combination with the CK6 promoter, is highly efficient and selective for delivering genes to ischemic skeletal muscle following systemic delivery.

### Discussion

### Example 2

PAD is a major health, care problem and more than a decade of clinical trials of gene therapy for PAD has failed to bring this approach forward in any meaningful way. Some of the plausible explanations for previous failures in human studies include: gene delivery vectors with inherently low magnitudes and durations of gene expression, and intra-muscular injection methods which are effective in pre-clinical studies with limited muscle mass and where most of the muscle is accessible to the needle. In humans, studies have found no evidence of transgene expression or when present was limited and heterogeneous in distribution. Therefore, systemic delivery offers numerous theoretical advantages for treating patients with PAD, but two major concerns exist. First, blood flow to the ischemic limb is reduced in PAD and this may limit access of the vector to ischemic tissue. Second, it is desirable to restrict gene expression to the cell type of interest since the expression of therapeutic genes in off-target tissues could potentially lead to deleterious side effects. The results of the current study show, for the first time, that gene expression in ischemic hindlimb muscle can be achieved by systemic injection of an AAV-based vector system with a skeletal muscle-tropic capsid (AAV-9) and a tissue-specific promoter (a compact version of the muscle-specific MCK promoter/enhancer). In the present study, using an AAV serotype 9-based vector in an adult mouse model of hindlimb ischemia (HLI), we demonstrate that: 1) the CMV promoter is adequate to achieve ischemia-tropic gene expression in skeletal muscle following intravenous administration; 2) the CK6 promoter provides for more robust and highly specific gene expression in ischemic skeletal muscle; 3) desialylation of cell surface glycans is increased in post-ischemic hindlimbs; 4) AAV-9 mediated gene expression in skeletal muscle is significantly increased following local desialylation of myofibers with neuraminidase; and 5) AAV-9 vector genome copy numbers and luciferase protein expression were both significantly higher in ischemic tissues as compared with the same vector genome packaged in an AAV-1 capsid (which, in contrast to AAV-9, requires sialic acid residues on galactosylated N-glycans for efficient cell surface binding and entry). Findings 3 and 4 are complementary, and strongly implicate desialylation as a mechanism contributing to the preferential transduction of ischemic muscle tissue following intravenous delivery. Taken together, these findings suggest two complementary mechanisms for the preferential transduction of ischemic muscle: increased vascular permeability and desialylation. In conclusion, ischemic muscle is preferentially targeted following systemic administration of AAV-9 in a mouse model of HLI. Unmasking of the primary AAV-9 receptor as a result of ischemia may contribute importantly to this effect.

Strong, non-selective, viral promoters such as CMV are typically used in animal studies as well as clinical trials of gene therapy for PAD. While tissue-specific promoters may be efficient at restricting gene expression to a particular cell or tissue type, their widespread use has not been realized because of a generally lower level of gene expression that is considered suboptimal for gene therapy applications. Furthermore, the “payload capacity” of the AAV capsid effectively limits the size of the recombinant AAV genome to approximately 5.3 kb. The choice of promoter for AAV-mediated, organ-specific gene expression should therefore be based on the size, specificity and strength of the promoter. Previous work in the field of gene therapy for muscular dystrophy led to the creation of hybrid promoter/enhancers in which various enhancers (including the MCK enhancer) have been introduced adjacent to the minimal MCK promoter. In a recent comparison of five such hybrid constructs, Hauser et al. identified a compact (571 bp) combination of the MCK enhancer and promoter (CK6) that was 6-fold stronger than the full-length 3.3-kb MCK promoter/enhancer and almost 12% as strong as the CMV promoter in muscle cells. Accordingly, we used the minimal CK6 promoter/enhancer in this study to achieve high-level, muscle-specific gene expression. Finally, in gene therapy protocols, the viral vector burden should be kept to a minimum to avoid vector-related side effects. While the specificity of gene expression needed for clinical efficacy will depend largely upon the nature of the therapeutic transgene, this study achieved efficient transduction of ischemic skeletal muscle without detectable adverse effects using a dose of 1.4×10¹³ vg/kg, which is comparable to intravenous doses of AAV vectors use in other small and large animal studies.

One might anticipate lower expression levels in ischemic limbs compared to the non-ischemic limbs based on the fact that ischemic limbs in this study had approximately one-half of the relative perfusion compared to non-ischemic limbs. Contrary to this expectation, the luciferase reporter gene and in vivo bioluminescence imaging (IVIS) clearly indicated that ischemic hindlimbs had higher luciferase activity than non-ischemic hindlimbs following intravenous delivery (Example 2, FIGS. 2b, c, f, and g). The ratios of in vitro luciferase activity in the ischemic skeletal muscle versus the other key organs such as liver and heart are summarized in Example 2, FIGS. 2e, i. Using the CK6 promoter, luciferase activity in the ischemic gastroenemius (GA) muscle was found to be −34-fold higher than in the contralateral GA and ˜150-fold higher than in the liver. Luciferase activity in the ischemic GA was also ˜2-fold higher while that in the liver was ˜42-fold lower in the CK6 group when compared to the CMV group. By combining the CK6 promoter with the AAV-9 capsid, we were able to harness the superior transduction efficiency of the AAV-9 capsid while attaining a >150-fold specificity for ischemic hindlimb skeletal muscle over liver. Using in vivo bioluminescence imaging, similar data were obtained in a second inbred mouse strain (BALB/c), which have been previously documented to have extremely poor perfusion recovery after hind-limb ischemia. These results confirm that the preferential transduction of ischemic skeletal muscle was not restricted to one mouse strain.

To the best of our knowledge, our results are the first to show robust and homogeneous gene expression in ischemic limbs compared to non-ischemic (contralateral) limbs following systemic delivery of an AAV vector. In further comparing the CMV and CK6 promoters, we found that the apparent tropism for ischemic skeletal muscle was much more pronounced with the CK6 promoter. One plausible explanation for this observation is that the increased desialylation associated with ischemia may act in synergy with the natural muscle tropism of the AAV-9 capsid and the specificity of the CK6 promoter for skeletal muscle. The eGFP reporter gene was then used to characterize the distribution of gene expression and the rate of transduction in ischemic skeletal muscle after IV administration of AAV-9 vectors driven by the CMV and CK6 promoters. Using the CK6 promoter, the transduction rate in ischemic skeletal muscle was >50% at the dose used in this study. These results also demonstrated that AAV-9 achieves a relatively homogeneous distribution of gene expression in ischemic skeletal muscle after IV administration, particularly when deployed in combination with a muscle-specific promoter.

Recently, Shen et al. showed that N-linked glycans with terminal galactosyl residues serve as the primary receptor for AAV-9 in Chinese hamster ovary (CFIO) cells. While sialylated glycans serve as the cellular receptors for other AAV serotypes, it was the desialylation of the N-terminal galactosylated glycans that increased cell surface binding and infectivity of AAV-9. Using two lectins, MAL I (which binds to α2,3-sialylated glycans) and ECL (which binds to the desialylated galactose residues of cell surface glycans), we report here, for the first time, that ischemia markedly increases the desialylation of cell surface glycans in the mouse HLI model of PAD, suggesting a possible mechanism for the increase in transduction efficiency under ischemic conditions. Studies with neuraminidase pretreatment were then conducted to test the hypothesis that increased desialylation of the cell surface glycans may enhance the gene transfer efficiency of intravenous AAV-9. We report here, for the first time, that pretreatment of a muscle with neuraminidase does result in significantly higher AAV-9-mediated gene expression following systemic delivery. The ischemia-induced desialylation of galactosylated N-glycans unmasks the primary cellular receptor for AAV-9, thus promoting cell surface binding and transduction after IV injection, ultimately resulting in increased transgene expression in ischemic as compared to non-ischemic myofibers.

The present method are also useful for improving recovery from injury in skeletal muscle by using the AAV9 vector comprising an extracellular superoxide dismutase gene sequence (EcSOD) as described in Example 1 and as recently-demonstrated by Saqib et al. (J. Vase. Surg., 2011).

### Conclusions

### Example 2

This study shows for the first time that transgene expression is targeted to ischemic muscle following systemic administration of muscle-tropic AAV vectors. The specificity of ischemic skeletal muscle transduction can be further improved with the use of a muscle-specific promoter. Increased desialylation of the cell surface N-glycans is a mechanism that likely contributes to the ischemic enhancement of AAV-9 mediated gene transfer after systemic delivery. These findings will be of immediate utility in pre-clinical studies examining the role of various genes in the recovery from hindlimb ischemia, and may ultimately prove valuable in clinical gene therapy protocols targeting PAD. AAV9/CK6/Luc vector genome copy numbers were 6-fold higher in ischemic muscles than in non-ischemic muscles in the HLI model, whereas this trend was reversed when the same vector was packaged in the AAV 1 capsid (which binds sialylated, as opposed to desialylated glyeans), further underscoring the importance of desialylation in the ischemic enhancement of transduction displayed by AAV9.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY

### Example 2

1. Hirsch A T, Criqui M H, Treat-Jacobson D, Regensteiner J G, Creager M A, Olin J W et al. Peripheral arterial disease detection, awareness, and treatment in primary care. JAMA 2001; 286(11): 1317-24.
2. Norgren L, Hiatt W R, Dormandy J A, Nehler M R, Harris K A, Fowkes F G et al. Inter-Society Consensus for the Management of Peripheral Arterial Disease (TASC II). Eur J Vase Endovasc Surg 2007; 33 Suppi 1: S1-75.
3. Tongers J, Roncalli J G, Losordo D W. Therapeutic Angiogenesis for Critical Limb Ischemia Microvascular Therapies Coming of Age. Circulation 2008; 118(1): 9-16.
4. Jones W S, Annex B H. Growth factors for therapeutic angiogenesis in peripheral arterial disease. Curr Opin Cardiol 2007; 22(5): 458-63.
5. Ghosh R, Walsh S R, Tang T Y, Noorani A, Hayes P D. Gene therapy as a novel therapeutic option in the treatment of peripheral vascular disease: systematic review and meta-analysis. International Journal of Clinical Practice 2008; 62(9): 1383-90.
6. Snyder R O, Miao C H, Patijn G A, Spratt S K, Danos O, Nagy D et al. Persistent and therapeutic concentrations of human factor IX in mice after hepatic gene transfer of recombinant AAV vectors. Nat Genet. 1997; 16(3): 270-276.
7. Flotte T R, Afione S A, Conrad C, McGrath S A, Soiow R, Oka H et al. Stable in vivo expression of the cystic fibrosis transmembrane conductance regulator with an adeno-associated virus vector. Proceedings of the National Academy of Sciences of the United States of America 1993; 90(22): 10613-7.
8. Hernandez Y J, Wang J, Kearns W G, Loiler S, Poirier A, Flotte T R. Latent Adeno-Associated Virus Infection Elicits Humoral but Not Cell-Mediated Immune Responses in aNonhuman Primate Model. J. Virol. 1999; 73(10): 8549-8558.
9. Inagaki K, Fuess S, Storm T A, Gibson G A, McTiernan C F, Kay M A et al. Robust Systemic Transduction with AAV9 Vectors in Mice: Efficient Global Cardiac Gene Transfer Superior to That of AAV8. Mol Ther 2006; 14(1): 45-53.
10. Bostick B, Ghosh A, Yue Y, Long C, Duan D. Systemic AAV-9 transduction in mice is influenced by animal age but not by the route of administration. Gene Therapy 2007; 14(22): 1605-9.
11. Reynolds P N, Nicklin S A, Kaliberova L, Boatman B G, Grizzle W E, Balyasmkova I V et al. Combined transductional and transcriptional targeting improves the specificity of transgene expression in vivo. Nat Biotech 2001; 19(9): 838-842,
12. Hauser M A, Robinson A, Hartigan-O'Connor D, Williams-Gregory D A, Buskin J N, Apone S et al. Analysis of muscle creatine kinase regulatory elements in recombinant adenoviral vectors. Molecular Therapy: the Journal of the American Society of Gene Therapy 2000; 2(1): 16-25.
13. Gregorevic P, Blankinship M J, Allen J M, Crawford R W, Meuse L, Miller D G et al. Systemic delivery of genes to striated muscles using adeno-associated viral vectors. Nat Med 2004; 10(8): 828-834.
14. Saiva M Z, Himeda C L, Tai P W L, Nishiuchi E, Gregorevic P, Allen J M et al. Design of Tissue-specific Regulatory Cassettes for High-level rAAV-mediated Expression in Skeletal and Cardiac Muscle. Mol Ther 2007; 15(2): 320-329.
15. Wang B, Li J, Fu F H, Chen C, Zhu X, Zhou L et al. Construction and analysis of compact muscle-specific promoters for AAV vectors. Gene Therapy 2008; 15(22): 1489-99.
16. Shen S, Bryant K D, Brown S M, Randell S H, Asokan A, Terminal N-linked galactose is the primary receptor for adeno-associated virus 9. J Biol Chem 2011; 286(15): 13532-40.
17. Creager M A, Olin J W, Belch J J F, Moneta G L, Henry T D, Rajagopalan S et al. Effect of Hypoxia-Inducible Factor-1 Gene Therapy on Walking Performance in Patients With Intermittent Claudication. Circulation 2011; 124(16): 1765-1773.
18. Rajagopalan S, Mohler E R, 3rd, Lederman R J, Mendelsohn F O, Saucedo J F, Goldman C K et al. Regional angiogenesis with vascular endothelial growth factor in peripheral arterial disease: a phase II randomized, double-blind, controlled study of adenoviral delivery of vascular endothelial growth factor 121 in patients with disabling intermittent claudication. Circulation 2003; 108(16): 1933-8.
19. Baumgartner I, Chronos N, Comerota A, Henry T, Pasquet J-P, Finiels F et al. Local Gene Transfer and Expression Following Intramuscular Administration of FGF-1 Plasmid DNA in Patients With Critical Limb Ischemia. Mol Ther 2009; 17(5): 914-921.
20. Byun J, Heard J M, Huh J E, Park S J, Jung E A, Jeong J O et al. Efficient expression of the vascular endothelial growth factor gene in vitro and in vivo, using an adeno-associated virus vector. J Mol Cell Cardiol 2001; 33(2): 295-305.
21. Pinkenburg O, Pfosser A, Hinkel R, Bottcher M, Dinges C, Lebherz C et al. Recombinant adeno-associated virus-based gene transfer of cathelicidin induces therapeutic neovascularization preferentially via potent collateral growth. Hum Gene Ther 2009; 20(2): 159-67.
22. M akin en K, Manninen H, Hedman M, Matsi P, Mussalo H, Aihava E et al. Increased vascularity detected by digital subtraction angiography after VEGF gene transfer to human lower limb artery: a randomized, placebo-controlled, double-blinded phase II study. Mol Ther 2002; 6(1): 127-33.
23. Dong J-Y, Fan P-D, Frizzell R A. Quantitative Analysis of the Packaging Capacity of Recombinant Adeno-Associated Virus. Human Gene Therapy 1996; 7(17): 2101-2112.
24. Grieger J C, Samulski R J. Packaging Capacity of Adeno-Associated Vims Serotypes: Impact of Larger Genomes on Infectivity and Postentry Steps. J. Virol. 2005; 79(15): 9933-9944.
25. Rodino-Klapac L R, Janssen P M L, Montgomery C L, Coley B D, Chicoine L G, Clark K R et al. A translational approach for limb vascular delivery of the micro-dystrophin gene without high volume or high pressure for treatment of Duchenne muscular dystrophy. J Trans Med. 2007; 5: 45.
26. Arruda V R, Fields P A, Milner R, Wainwright L, De Miguel M P, Donovan P J et al. Lack of germline transmission of vector sequences following systemic administration of recombinant AAV-2 vector in males, Mol Ther 2001; 4(6): 586-92.
27. Herzog R W, Fields P A, Arruda V R, Brubaker J O, Armstrong E, McClintock D et al. Influence of vector dose on factor IX-specific T and B cell responses in muscle-directed gene therapy. Hum Gene Ther 2002; 13(11): 1281-91.
28. Dokun A O, Keum S, Hazarika S, Li Y, Lamonte G M, Wheeler F et al. A Quantitative Trait Locus (LSq-1) on Mouse Chromosome 7 Is Linked to the Absence of Tissue Loss After Surgical Hindlimb Ischemia. Circulation 2008; 117(9): 1207-1215.
29. Prasad K-M, Xu Y, Yang Z, Acton S T, French B A. Robust Cardiomyocyte-Specific Gene Expression Following Systemic Injection of AAV: In Vivo Gene Delivery Follows a Poisson Distribution. Gene Therapy 2011; 18(1): 43-52,
30. Ried M U, Girod A, Leike K, Butting H, Hallek M. Adeno-associated virus capsids displaying immunoglobulin-binding domains permit antibody-mediated vector retargeting to specific cell surface receptors. Journal of Virology 2002; 76(9): 4559-66,
31. Prasad K-M R, Xu Y, Yang Z, Toufektsian M-C, Berr S S, French B A. Topoisomerase inhibition accelerates gene expression after adeno-associated virus-mediated gene transfer to the mammalian heart. Molecular Therapy: the Journal of the American Society of Gene Therapy 2007; 15(4): 764-71.
32. Li Y, Hazarika S, Xie D, Pippen A M, Kontos C D, Annex B H. In mice with type 2 diabetes, a vascular endothelial growth factor (VEGF)-activating transcription factor modulates VEGF signaling and induces therapeutic angiogenesis after hindlimb ischemia. Diabetes 2007; 56(3): 656-65.
33. Hazarika S, Dokun A O, Li Y, Popel A S, Kontos C D, Annex B H. Impaired angiogenesis after hindlimb ischemia in type 2 diabetes mellitus: differential regulation of vascular endothelial growth factor receptor 1 and soluble vascular endothelial growth factor receptor 1. Circ Res 2007; 101(9): 948-56.
34. Wu J C, Inubushi M, Sundaresan G, Schelbert H R, Gambhir S S. Optical imaging of cardiac reporter gene expression in living rats. Circulation 2002; 105(14): 1631-4.
35. Souza et al. (U.S. Pat. Pub. No. 2011/0212529, published Sep. 1, 2011).
36. Saqib et al, J. Vase. Surg., 2011, 54:3:810, Epub 2011 Jul. 2, AAV9-Mediated Overexpression of Extracellular Superoxide Dismutase Improves Recovery from Surgical Hind-limb Ischemia in BALB/c Mice.

### Example 3

Some of the sequences and vectors used herein are derived from prior work. For example, the CK6 experiments disclosed herein utilize sequences and vectors based in part on the work described in Hauser et al. (Analysis of muscle creatine kinase regulatory elements in recombinant adenoviral vectors. Molecular Therapy: the Journal of the American Society of Gene Therapy 2000; 2(1): 16-25). FIGS. 1 and 2 of Hauser et al. are also reproduced herein as Example 3, FIG. 1 and Example 3, FIG. Example 3, FIG. 1 demonstrates a restriction map schematic (Example. 3, FIG. 1a) and the sequence of the 206 bp MCK upstream enhancer (Example. 3, FIG. 1b) of the muscle creatine kinase promoter and enhancer (SEQ ID NO:15). The sequence of a 3355-bp genomic fragment of the murine MCK transcriptional regulatory region extends from −3348 to +7 relative to the transcriptional start site which has GenBank database Accession No. AF188002. GenBank database Accession No. AF188002 is provided as SEQ ID NO:4 herein. The sequence of the 206-bp MCK upstream enhancer (SEQ ID NO:15 herein), a fragment of the 3357 bp sequence of Accession No. AF188002, is shown, with protein binding sites underlined. The sequence alterations corresponding to the 2R and S5 modifications are indicated above the wild-type sequence. The NcoI site indicated marks the upstream boundary of the MEF2 deletion in construct CK5.

Example 3, FIG. 2 demonstrates schematically transcriptional regulatory cassettes based on the muscle creatine kinase promoter and enhancer. CK3 contains the full 3357-bp region extending from −3348 to +7 relative to the transcriptional start site. CK2 extends from −1256 to +7. CK5 contains part of the 2RS5 enhancer, extending from nucleotides −1256 to −1091, thereby deleting the enhancer MEF2 site, and a promoter extending from −944 to +7. Previous deletion studies indicated no control elements within the −1050 to −945 MCK promoter region that was deleted. CK6 contains the full 2RS5 enhancer sequence demonstrated in Example 3 FIG. 1 and a promoter extending from −358 to +7. CK4 contains the full 2RS5 enhancer and a promoter extending from −80 to +7. The CMV promoter used in plasmid constructs of Hauser extended from −525 to +1 relative to its transcriptional start site. All constructs of Hauser included the 150-bp minx intron, a nuclear-targeted lacZ transgene, and the SV40 polyadenylation signal. The schematic diagram at the bottom shows an expression cassette inserted into a recombinant adenoviral vector so that transcription proceeds away from the left viral ITR. MCK regulatory elements in this orientation direct muscle-specific expression, while those in the opposite orientation allow leaky transcription in nonmuscle cells.

What is claimed is:

1. A method of preventing or treating an injury, disease, or disorder in cardiac or skeletal muscle, said method comprising administering to a subject in need thereof a pharmaceutical composition comprising an effective amount of a recombinant adeno-associated viral (AAV) vector comprising a regulatory element active in muscle cells, wherein said regulatory element comprises at least one promoter element and optionally at least one enhancer element, further wherein said AAV vector comprises at least one gene operably linked to said at least one promoter, or active fragments, modifications, or homologs thereof, thereby preventing or treating an injury, disease, or disorder in cardiac or skeletal muscle.

2. The method of claim 1, wherein at least one promoter element is a tissue specific promoter.

3. The method of claim 1, wherein the AAV is AAVS (SEQ ID NO:11) or AAV9 (SEQ ID NOT).

4. The method of claim 1, wherein the at least one promoter element and the at least one enhancer element are from the same species of animal.

5. The method of claim 4, wherein the species is selected from group consisting of mouse, human, chicken, and rat.

6. The method of claim 1 wherein the vector is AcTnTEcSOD.

7. The method of claim 1, wherein the at least one promoter is selected from the group consisting of a cardiac troponin-T promoter, a muscle creatine kinase promoter, and a desmin promoter.

8. The method of claim 1, wherein an effective amount of neuraminidase or other desialylation agent is administered to said subject before administration of said AAV vector.

9. The method of claim 1, wherein said injury, disease, or disorder is selected from the group consisting of myocardial infarction, reperfusion injury, heart failure, and peripheral artery disease.

10. The method of claim 1, wherein said gene is a therapeutic gene.

11. The method of claim 1 wherein said AAV is AAV9 and comprises the sequence of SEQ ID NO:1, said at least one promoter comprises the sequence of SEQ ID NO:4, 16, 17, or 18 or the 365 bp proximal promoter region of muscle creatine kinase extending from nucleotide position −358 to +7 relative to the transcriptional start site, said at least one optional enhancer comprises the sequence of SEQ ID NO:15, and said at least one therapeutic gene comprises the sequence of SEQ ID NO:12 or 14.

12. The method of claim 1, wherein said method inhibits ventricular remodeling and heart failure associated with myocardial infarction and ischemia.

13. The method of claim 12, wherein when said AAV vector comprises an extracellular superoxide dismutase 3 (EcSOD) sequence of SEQ ID NO:12 or 14, said administration results in increased expression or activity of extracellular superoxide dismutase 3 in the heart.

14. The method of claim 13, wherein said expression or activity is in cardiomyocytes.

15. The method of claim 1, wherein said pharmaceutical composition is administered prior to, simultaneous with, or after a surgical procedure.

16. The method of claim 1, wherein said subject is a human.

17. The method of claim 1, wherein said pharmaceutical composition is administered systemically, intravenously, by intracoronary infusion, locally, or by direct injection into myocardium.

18. The method of claim 1, wherein said subject is pretreated with an effective amount of neuraminidase or other desialylation agent to increase desialylation of cell surface N-linked glycans and enhance AAV binding to its cognate receptor.

19. The method of claim 18, wherein said neuraminidase or other desialylation agent is applied systemically or locally.

20. The method of claim 1, wherein said regulatory element is a 571 bp CK6 muscle creatine kinase enhancer/promoter regulatory element, wherein said 571 bp enhancer/promoter consists of the 206 bp sequence of SEQ ID NO:16 and the 365 bp proximal promoter region of the muscle creatine kinase genomic fragment having GenBank Accession No. API 88002, wherein said 365 bp proximal promoter region extends from nucleotide position −358 to +7 relative to the transcriptional start site.

21. The method of claim 1, wherein a capsid gene sequence of said AAV is used.

22. The method of claim 21, wherein said regulatory element increases expression of said gene in said cardiac or skeletal muscle.

23. The method of claim 22, wherein said expression is in a cardiac myocyte or in a skeletal muscle myocyte.

24. The method of claim 2, wherein said tissue is muscle.

25. The method of claim 21, wherein said AAV is AAV9 and said capsid gene sequence comprises nucleotide residue positions 2116 to 4329 of SEQ ID NO:1.

26. The method of claim 1, wherein said AAV vector preferentially targets cardiac muscle or skeletal muscle.

27. The method of claim 26, wherein said AAV vector preferentially targets an ischemic region.

28. The method of claim 27, wherein said ischemic region is an infarct border zone.

29. The method of claim 26, wherein said AAV vector preferentially targets eardiomyoeytes or skeletal myocytes.

30. A method of targeting and transducing muscle with an AAV vector, said method comprising administering to a subject a pharmaceutical composition comprising an effective amount of a recombinant adeno-associated viral (AAV) vector comprising a regulatory element, wherein said regulatory element comprises at least one promoter element and optionally at least one enhancer element, further wherein said AAV vector optionally comprises at least one gene operably linked to said at least one promoter element, or active fragments, modifications, or homologs thereof, thereby targeting and transducing muscle with an AAV vector.

31. The method of claim 30 wherein said AAV vector preferentially targets skeletal muscle.

32. The method of claim 30, wherein the AAV is AAV8 (SEQ ID NO:11) or AAV9(SEQ ID NO:1).

33. The method of claim 30, wherein said subject is pretreated with an effective amount of neuraminidase or other desialylation agent to increase desialylation of cell surface N-linked glycans and enhance AAV binding to its cognate receptor.

34. The method of claim 30, wherein said regulatory element is a 571 bp CK6 muscle creatine kinase enhancer/promoter regulatory element, wherein said 571 bp enhancer/promoter consists of the 206 bp sequence of SEQ ID NO:16 and the 365 bp proximal promoter region of the muscle creatine kinase genomic fragment having GenBank Accession No. API 88002, wherein said 365 bp proximal promoter region extends from nucleotide position −358 to +7 relative to the transcriptional start site.

35. The method of claim 30, wherein said at least one promoter comprises the sequence of SEQ ID NOs:4, 16, 17, or 18 or the 365 bp proximal promoter region of muscle creatine kinase extending from nucleotide position −358 to +7 relative to the transcriptional start site, said at least one optional enhancer comprises the sequence of SEQ ID NO:15, and said at least one therapeutic gene comprises the sequence of SEQ ID NO:12 or 14.

36. The method of claim 1, wherein said AAV vector comprises a sequence encoding an siRNA or an miRNA.
